(19) United States
(12) Patent Application Publication
(10) Pub. No.: US 2016/0113893 A1
(43) Pub. Date: Apr. 28, 2016

(54) NEW THERAPEUTIC USES OF ENZYME INHIBITORS

(71) Applicant: PROXIMAGEN LIMITED, Cambridge (GB)

(72) Inventors: Kenneth Mulvany, London (GB); Martyn Pritchard, St. Ives, Cambridgeshire (GB)

(21) Appl. No.: 14/895,705

(22) PCT Filed: Jun. 12, 2014
(86) PCT No.: PCT/GB2014/051817

§ 371 Date: Dec. 3, 2015

(30) Foreign Application Priority Data
Jun. 12, 2013 (GB) 1310488.0
Dec. 12, 2013 (GB) 1322036.3

(51) Int. Cl. A61K 31/165

(52) U.S. Cl. CPC A61K 31/165

(57) ABSTRACT

The invention relates to use of compounds which inhibit VAP-1/SSAO activity for the treatment of muscular dystrophy.

### FIELD OF THE INVENTION

This invention relates to the use of a compound which inhibits VAP-1/SSAO activity for the treatment of muscular dystrophy. The invention also relates to the use of pharmaceutical compositions comprising these compounds for the treatment of muscular dystrophy.

### BACKGROUND ART

Semicarbazide-sensitive amine oxidase (SSAO), otherwise known as Vascular Adhesion Protein-1 (VAP-1) or Amine Oxidase, Copper Containing 3 (AOC3), belongs to the copper-containing amine oxidase family of enzymes (EC.1.4.3.6). Members of this enzyme family are sensitive to inhibition by semicarbazide and utilize cupric ion and protein-derived topa quinone (TPQ) cofactor in the oxidative deamination of primary amines to aldehydes, hydrogen peroxide, and ammonia according to the following reaction:

R—CH₂—NH₂+O₂→R—CHO+H₂O₂+NH₃

Known substrates for human SSAO include endogenous methylamine and aminoacetone as well as some xenobiotic amines such as benzylamine [Lyles, Int. J. Biochem. Cell Biol. 1996, 28, 259-274; Klinman, Biochim. Biophys. Acta 2003, 1647(1-2), 131-137; Matyus et al., Curr. Med. Chem. 2004, 11(10), 1285-1298; O'Sullivan et al., Neurotoxicology 2004, 25(1-2), 303-315]. In analogy with other copper-containing amine oxidases, DNA-sequence analysis and structure determination suggest that the tissue-bound human SSAO is a homodimeric glycoprotein consisting of two 90-100 kDa subunits anchored to the plasma membrane by a single N-terminal membrane spanning domain [Morris et al., J. Biol. Chem. 1997, 272, 9388-9392; Smith et al., J. Exp. Med. 1998, 188, 17-27; Airenne et al., Protein Science 2005, 14, 1964-1974; Jakobsson et al., Acta Crystallogr. D Biol. Crystallogr. 2005, 61(Pt 11), 1550-1562].

SSAO activity has been found in a variety of tissues including vascular and non-vascular smooth muscle tissue, endothelium, and adipose tissue [Lewinsohn, Braz. J. Med. Biol. Res. 1984, 17, 223-256; Nakos & Gossrau, Folia Histochem. Cytobiol. 1994, 32, 3-10; Yu et al., Biochem. Pharmacol. 1994, 47, 1055-1059; Castillo et al., Neurochem. Int. 1998, 33, 415-423; Lyles & Pino, J. Neural. Transm. Suppl. 1998, 52, 239-250; Jaakkola et al., Am. J. Pathol. 1999, 155, 1953-1965; Morin et al., J. Pharmacol. Exp. Ther. 2001, 297, 563-572; Salmi & Jalkanen, Trends Immunol. 2001, 22, 211-216]. In addition, SSAO protein is found in blood plasma and this soluble form appears to have similar properties as the tissue-bound form [Yu et al., Biochem. Pharmacol. 1994, 47, 1055-1059; Kurkijarvi et al., J. Immunol. 1998, 161, 1549-1557]. It has recently been shown that circulating human and rodent SSAO originates from the tissue-bound form [Göktürk et al., Am. J. Pathol. 2003, 163(5), 1921-1928; Abella et al., Diabetologia 2004, 47(3), 429-438; Stolen et al., Circ. Res. 2004, 95(1), 50-57], whereas in other mammals the plasma/serum SSAO is also encoded by a separate gene called AOC4 [Schwelberger, J. Neural. Transm. 2007, 114(6), 757-762].

The precise physiological role of this abundant enzyme has yet to be fully determined, but it appears that SSAO and its reaction products may have several functions in cell signalling and regulation. For example, recent findings suggest that SSAO plays a role in both GLUT4-mediated glucose uptake [Enrique-Tarancon et al., J. Biol. Chem. 1998, 273, 8025-8032; Morin et al., J. Pharmacol. Exp. Ther. 2001, 297, 563-572] and adipocyte differentiation [Fontana et al., Biochem. J. 2001, 356, 769-777; Mercier et al., Biochem. J. 2001, 358, 335-342]. In addition, SSAO has been shown to be involved in inflammatory processes where it acts as an adhesion protein for leukocytes [Salmi & Jalkanen, Trends Immunol. 2001, 22, 211-216; Salmi & Jalkanen, in “Adhesion Molecules: Functions and Inhibition” K. Ley (Ed.), 2007, pp. 237-251], and might also play a role in connective tissue matrix development and maintenance [Langford et al., Cardiovasc. Toxicol. 2002, 2(2), 141-150; Goktirk et al., Am. J. Pathol. 2003, 163(5), 1921-1928]. Moreover, a link between SSAO and angiogenesis has recently been discovered [Noda et al., FASEB J. 2008, 22(8), 2928-2935].

Several studies in humans have demonstrated that SSAO activity in blood plasma is elevated in conditions such as congestive heart failure, diabetes mellitus, Alzheimer's disease, and inflammation [Lewinsohn, Braz. J. Med. Biol. Res. 1984, 17, 223-256; Boomsma et al., Cardiovasc. Res. 1997, 33, 387-391; Ekblom, Pharmacol. Res. 1998, 37, 87-92; Kurkijarvi et al., J. Immunol. 1998, 161, 1549-1557; Boomsma et al., Diabetologia 1999, 42, 233-237; Meszaros et al., Eur. J. Drug Metab. Pharmacokinet. 1999, 24, 299-302; Yu et al., Biochim. Biophys. Acta 2003, 1647(1-2), 193-199; Matyus et al., Curr. Med. Chem. 2004, 11(10), 1285-1298; O'Sullivan et al., Neurotoxicology 2004, 25(1-2), 303-315; del Mar Hernandez et al., Neurosci. Lett. 2005, 384(1-2), 183-187]. The mechanisms underlying these alterations of enzyme activity are not clear. It has been suggested that reactive aldehydes and hydrogen peroxide produced by endogenous amine oxidases contribute to the progression of cardiovascular diseases, diabetic complications and Alzheimer's disease [Callingham et al., Prog. Brain Res. 1995, 106, 305-321; Ekblom, Pharmacol. Res. 1998, 37, 87-92; Yu et al., Biochim. Biophys. Acta 2003, 1647(1-2), 193-199; Jiang et al., Neuropathol Appl Neurobiol. 2008, 34(2), 194-204]. Furthermore, the enzymatic activity of SSAO is involved in the leukocyte extravasation process at sites of inflammation where SSAO has been shown to be strongly expressed on the vascular endothelium [Salmi et al., Immunity 2001, 14(3), 265-276; Salmi & Jalkanen, in “Adhesion Molecules: Functions and Inhibition” K. Ley (Ed.), 2007, pp. 237-251]. Accordingly, inhibition of SSAO has been suggested to have a therapeutic value in the prevention of diabetic complications and in inflammatory diseases [Ekblom, Pharmacol. Res. 1998, 37, 87-92; Salmi et al., Immunity 2001, 14(3), 265-276; Salter-Cid et al., J. Pharmacol. Exp. Ther. 2005, 315(2), 553-562].

SSAO knockout animals are phenotypically overtly normal but exhibit a marked decrease in the inflammatory responses evoked in response to various inflammatory stimuli [Stolen et al., Immunity 2005, 22(1), 105-115]. In addition, antagonism of its function in wild type animals in multiple animal models of human disease (e.g. carrageenan-induced paw inflammation, oxazolone-induced colitis, lipopolysaccharide-induced lung inflammation, collagen-induced arthritis, endotoxin-induced uveitis) by the use of antibodies and/or small molecules has been shown to be protective in decreasing the leukocyte infiltration, reducing the severity of the disease phenotype and reducing levels of inflammatory cytokines and chemokines [Kirton et al., Eur. J. Immunol. 2005, 35(11), 3119-3130; Salter-Cid et al., J. Pharmacol. Exp. Ther. 2005, 315(2), 553-562; McDonald et al., Annual Reports in Medicinal Chemistry 2007, 42, 229-243; Salmi & Jalkanen, in “Adhesion Molecules: Functions and Inhibition” K. Ley (Ed.), 2007, pp. 237-251; Noda et al., FASEB J. 2008 22(4), 1094-1103; Noda et al., FASEB J. 2008, 22(8), 2928-2935]. This anti-inflammatory protection seems to be afforded across a wide range of inflammatory models all with independent causative mechanisms, rather than being restricted to one particular disease or disease model. This would suggest that SSAO may be a key nodal point for the regulation of the inflammatory response, and it seems therefore likely that SSAO inhibitors may be effective anti-inflammatory drugs in a wide range of human diseases.

Fibrosis can result from chronic tissue inflammation when the resolution of the inflammation is partly abrogated by the chronic nature of the inflammatory stimulus. The result can be inappropriate repair of the tissue with excessive extracellular matrix deposition (including collagen) with tissue scarring. This is a consequence of myofibroblast activation by stimuli including fibronectin and reactive oxygen species as well as growth factors such as transforming growth factor-β-1 (TGFβ-1), insulin-like growth factor-I (IGF-I), platelet-derived growth factor (PDGF) and connective tissue growth factor (CTGF) resulting in increased production of collagen, elastin, hyaluronan, glycoproteins and proteoglycans. In addition the activity of invading macrophages plays a crucial part in regulating the repair and fibrotic processes.

VAP-1 has also been implicated in the progression and maintenance of fibrotic diseases especially in the liver. Weston and Adams (J Neural Transm. 2011, 118(7), 1055-64) have summarised the experimental data implicating VAP-1 in liver fibrosis. Weston et al (EASL Poster 2010) showed highly increased expression of VAP-1 in human fibrotic liver, particularly associated with the activated myofibroblasts and collagen fibrils. This anatomical association with fibrosis was consistent with the observation that blockade of VAP-1 accelerated the resolution of carbon tetrachloride induced fibrosis, and suggested a role for the VAP-1/SSAO enzyme product H2O2 in the activation of the myofibroblasts. The same authors also showed that the pro-fibrotic growth factor TGFβ increased the expression of VAP-1 in liver cells by approximately 50-fold.

There are a large number of diseases which cause wasting or atrophy of muscles and some of these are associated with significant amounts of fibrosis. The most well-known include Duchenne Muscular Dystrophy and Becker Muscular Dystrophy. These are both caused by defects in the muscle cytoskeletal protein dystrophin, and in the former usually results in death by the age of 25, while in the less severe Becker form, patients usually survive into old age. The pathological basis of both these diseases is considered to be a consequence of poor muscle cell connectivity to the extracellular matrix, resulting in the weakening of the sarcolemma and cell death. The muscle tissue then suffers from repeated cycles of cell death and aberrant repair, resulting in fibrosis and the replacement of muscle tissue by fat tissue (Mann et al., 2011, Skeletal Muscle. 1(1):21; Klinger et al. 2012 Acta Myol. 31(3): 184-189). The symptoms of these diseases include pain and muscle weakness. Other dystrophies arising from similar causes include limb girdle muscular dystrophy, congenital muscular dystrophy and distal muscular dystrophy. All of these appear to have defects in cell attachment to the extracellular matrix. Fibrosis is therefore a major issue in the muscular dystrophies and a therapeutic capable of reducing or reversing the fibrosis would be extremely beneficial to patients suffering from muscular dystrophy.

### DETAILED DESCRIPTION OF THE INVENTION

The invention described herein relates to the expression of VAP-1 in dystrophic muscle, which VAP-1 expression is expected to increase during the fibrotic disease process. In normal muscle the expression of VAP-1 is low, and largely restricted to the blood vessels (Salmi et al., 1993, J. Exp. Med. 178, 2255-2260) but increases in inflamed and fibrotic tissues. This increase in expression in the diseased state makes VAP-1 a promising therapeutic target in dystrophic muscle. Inhibition of VAP-1/SSAO is expected to reduce the concentration of pro-inflammatory and pro-fibrotic enzyme products (such as aldehydes, hydrogen peroxide and ammonia) whilst also decreasing the adhesive capacity of immune and myofibroblast cells and correspondingly their activation and invasion of the muscle tissue. Thus inhibition of VAP-1/SSAO is expected to be therapeutically beneficial in the treatment of muscle fibrosis and therefore muscular dystrophy.

In addition to treating the fibrosis component of muscular dystrophy, inhibition of VAP-1/SSAO is expected to have further beneficial effects on muscle tissue maintenance. VAP-1/SSAO inhibitors are known to reduce leukocyte and monocyte incursion into tissues. It is known from the mdx mouse model, a murine model of Duchenne Muscular Dystrophy, that partial inhibition of macrophage incursion into the muscle tissue has a beneficial effect on muscle tissue maintenance. Therefore VAP-1/SSAO inhibitors are expected to have therapeutic effects in dystrophic muscle by reducing leukocyte, and particularly monocyte, incursion into the tissue.

In summary, it is expected that VAP-1/SSAO inhibitors will reduce inflammation and muscle loss through inhibition of leukocyte invasion, and reduce muscular fibrosis and scarring through reduced VAP-1 activity in the muscle tissue, and reduce inflammatory and fibrotic cell activation in muscle tissue through reduced production of pro-inflammatory and pro-fibrotic enzyme products such as aldehydes, hydrogen peroxide and ammonia.

According to the present invention, there is provided a VAP-1 inhibitor for use in the treatment of muscular dystrophy.

In another aspect, the invention provides the use of a VAP-1 inhibitor in the manufacture of a medicament for treatment of muscular dystrophy.

In another aspect, the invention provides a method of treating muscular dystrophy comprising administering to a subject suffering such disease an effective amount of a VAP-1 inhibitor.

As used herein, the terms “treatment,” “treating,” “treat” and the like, refer to obtaining a desired pharmacologic and/or physiologic effect. The effect can be prophylactic in terms of completely or partially preventing muscular dystrophy or a symptom thereof and/or can be therapeutic in terms of a partial or complete cure for muscular dystrophy and/or an adverse effect attributable to the disease. “Treatment,” as used herein, covers any treatment of muscular dystrophy in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

An “effective amount” of a VAP-1 inhibitor refers to the amount of a VAP-1 inhibitor that, when administered to a mammal or other subject for treating muscular dystrophy, is sufficient to effect such treatment for the disease. The “effective amount” will vary depending on the VAP-1 inhibitor, the disease and its severity and the age, weight, etc., of the subject to be treated.

The term “VAP-1 inhibitor” or “VAP-1 inhibitor compound” includes both non-biological small molecule inhibitors of VAP-1 and biological inhibitors of VAP-1, including but not limited to RNA, antibodies, polypeptidic or proteinaceous inhibitors of VAP-1.

For present purposes, a “VAP-1 inhibitor” or “VAP-1 inhibitor compound” is one which has an IC50 value of less than 1000 nM in the VAP-1 Assay described below.

### VAP-1 Inhibitors

Small molecules of different structural classes have previously been disclosed as VAP-1 inhibitors, for example in WO 02/38153 (tetrahydroimidazo[4,5-c]pyridine derivatives), in WO 03/006003 (2-indanylhydrazine derivatives), in WO 2005/014530 (allylhydrazine and hydroxylamine (aminooxy) compounds) and in WO 2007/120528 (allylamino compounds), WO2011034078 (N-[3-(heterocyclyl or phenyl)benzyl]-2-aminoglycinamides), and WO2012120195 (Pyridazinones), and WO2012124696 (Guanidines), and Bioorganic & Medicinal Chemistry (2013), 21(13), 3873-3881 (1H-imidazol-2-amine derivatives), and Bioorganic & Medicinal Chemistry (2013), 21(5), 1219-1233 (Thiazoles).

Many further small molecule VAP-1 inhibitors are known, for example, haloallyl amines of WO2009066152; imidazopyridines of WO2010064020; dihydralazine (WO2010015870); pyrazolo[4,3-c]pyridines of WO2010031791; 4,5,6,7-tetrahydroimidazo[4,5-c]pyridines of US2002198189, WO0238153 and WO2010031789; oximes of WO2010029379; allyl hydrazine, hydroxylamine and other compounds of US2005096360, WO2006094201 and WO2005014530; amine, amide and allylamino compounds of WO2007120528, US2007078157, WO2005082343 and WO2009055002; hydroxamic acids of WO2006013209; vitamin B1, vitamin B1 derivatives and vitamin B1 precursors of WO2008025870; 2,3,4,6,8-pentamethoxyl-dibenzofuran (CN100486971); compounds of US2007066646; aminoglycosides of WO2005063261; carbocyclic hydrazino compounds of WO03006003; hydrazono compounds of US2004106654 and WO0202090; haloallylamines such as MDL72161A, MDL72274A and MDL72964A (mofegiline, (E)-4-fluoro-beta-fluoromethylene benzene butanamine hydrochloride, (E)-2-(4-fluorophenethyl)-3-fluoroallylamine hydrochloride) as in WO9323023, Lyles et al, Biochem. Pharmacol., 1987, 2847 and McDonald et al, J. Med. Chem., 1985, 186; thiazoles of WO2004087138, WO2004067521, WO2005089755, WO2006011631 and WO2006028269; semicarbazide and hydrazines (e.g. phenylhydrazine, phenelzine, carbazine and hydrazaline) as in McDonald et al, Annual reports in medicinal chemistry, 42, 229-243, 2007; hydrazines of WO2004104191 and WO2002002541; 1,3,4-oxadiazine compounds of W0200202541; hydrazino alcohol derivatives of WO2005080319; propargylamines of Sayre et al, Biochem., Biophys., Res. Commun, 2003, 788, Sayre et al, Bioorg. Med. Chem., 2006, 1444 and Sayre et al, Eur. J. Biochem., 2002, 3645; peptides as in Yegutkin, Eur. J. Immunol., 2004, 2276; dihydropyrroles of US20060025438 and Sayre et al, J. Am. Chem. Soc., 2002, 12135; proline amide derivatives of Sayre et al, Bioorg. Med. Chem., 2007, 1868; benzene and thiophene derivatives of WO2009145360 and WO 2009096609; thiazoles of US20040259923; and also includes molecules such as 5-hydroxytryptamine, 3-bromopropylamine, N-(phenyl-allyl)-hydrazine HCl (LJP-1207), 2-hydrazinopyridine, MDL-72274 ((E)-2-phenyl-3-chloroallylamine hydrochloride), MDL-72214 (2-phenylallylamine), MDL-72145, MDL-72161, mexiletine, isoniazid, imipramine, maprotiline, zimeldine, nomifensine, azoprocarbazine, monomethylhydrazine, dl-alphamethyltryptamine, dl-alphamethylbenzylamine, MD780236 (Dostert et al, J. Pharmacy & Pharmacol., 1984, 782), Z-3-Fluoro-2-(4-methoxybenzyl)allylamine hydrochloride (LJP-1586) (O'Rourke et al, JPET, 2008, 867), 2-(dimethyl(2-phenylethyl)silyl)methanamine, cuprozine, alkylamino derivatives of 4-aminomethylpyridine (Bertini et al, J. Med. Chem., 2005, 664), (1S,2S)-2-(1-methylhydrazino)-1-indanol (BTT-2052) (Marttila-lchihara et al, JI, 2010, 2741), RTU-1096, kynuramine and carbidopa.

Biological inhibitors of VAP-1 include but are not limited to antibodies to VAP-1, RNAi, siRNA (examples of siRNAs suitable for targeting VAP-1 are described, for example, in WO2006134203), anti-sense oligonucleotides, anti-sense peptidyl nucleic acids, and aptamers. Examples of VAP-1 antibodies include but are not limited to anti-VAP-1 neutralizing antibody (available, for example, from R&D systems, Minneapolis, Minn., catalogue numbers. AF3957, MAB39571 and MAB3957; Everest Biotech, Oxford, UK, catalogue number EB07582; and antibodies identified in WO2008129124, WO2003093319 and Koskinen et al, Blood, 2004, 3388, Arvilommi et al, Eur. J. Immunol., 1996, 825, Salmi et al, J. Exp. Med., 1993, 2255 and Kirten et al, Eur. J. Immunol., 2005, 3119.

The VAP-1 inhibitors disclosed specifically or generically in the above publications are expected to have utility in the treatment of muscular dystrophy according to the present invention.

Specific Examples of VAP-1 inhibitor compounds suitable for use in the present invention are provided below. Any pharmaceutically acceptable salt form of the Examples is suitable for use in the present invention. Specific examples of inhibitors of VAP-1 include the compounds specifically disclosed as Examples in WO 2010/031789, namely:

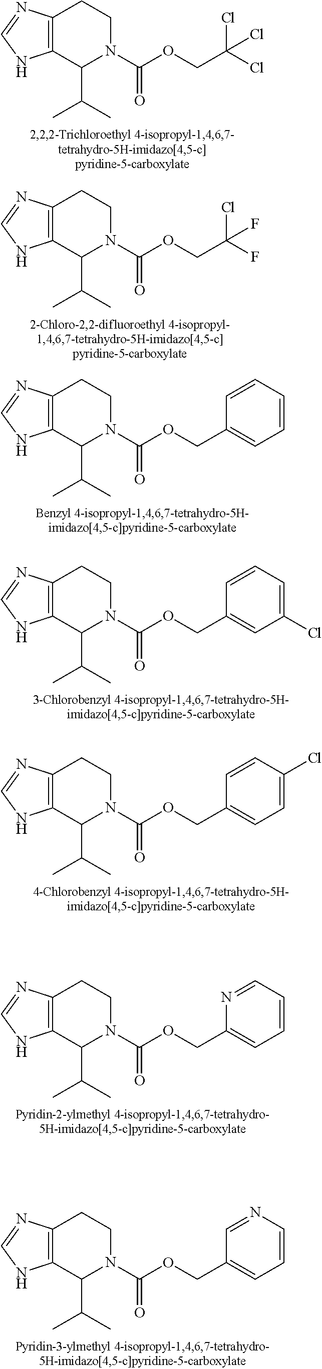

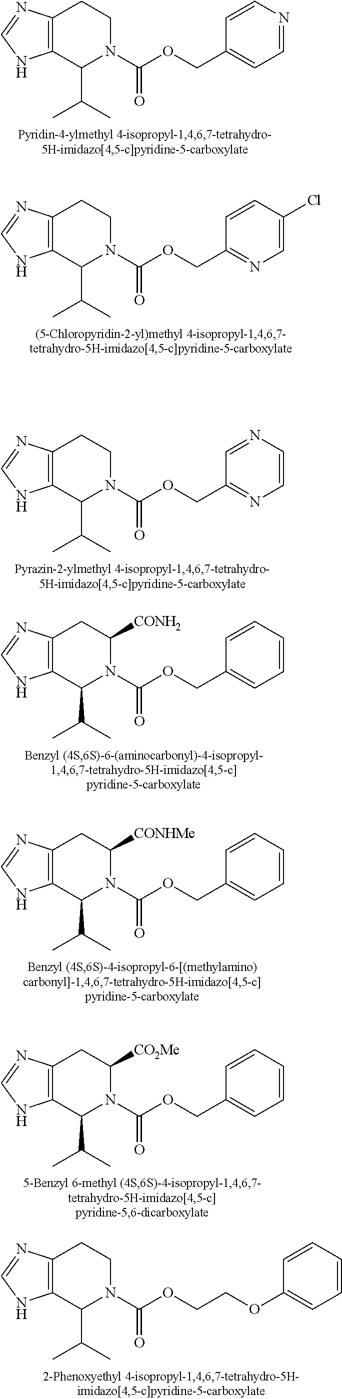

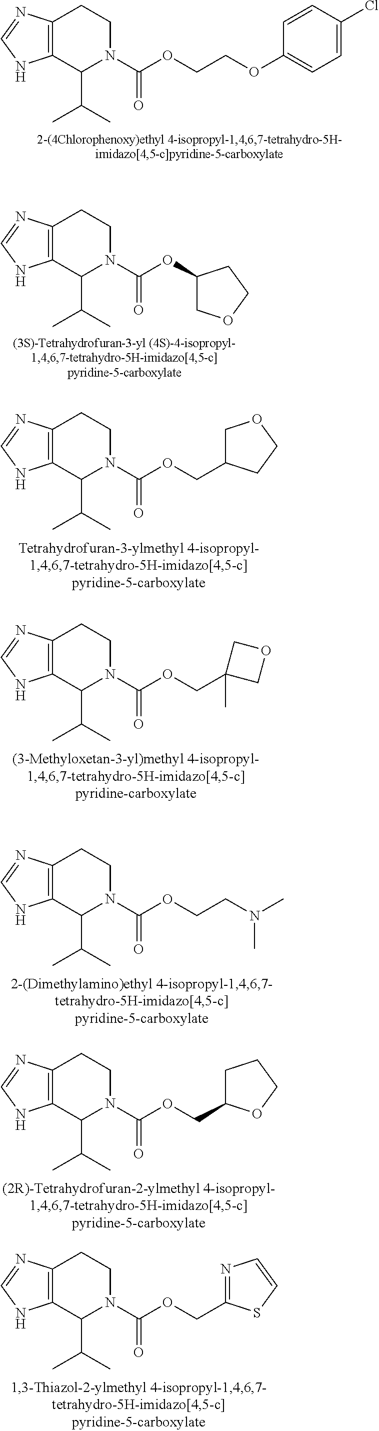

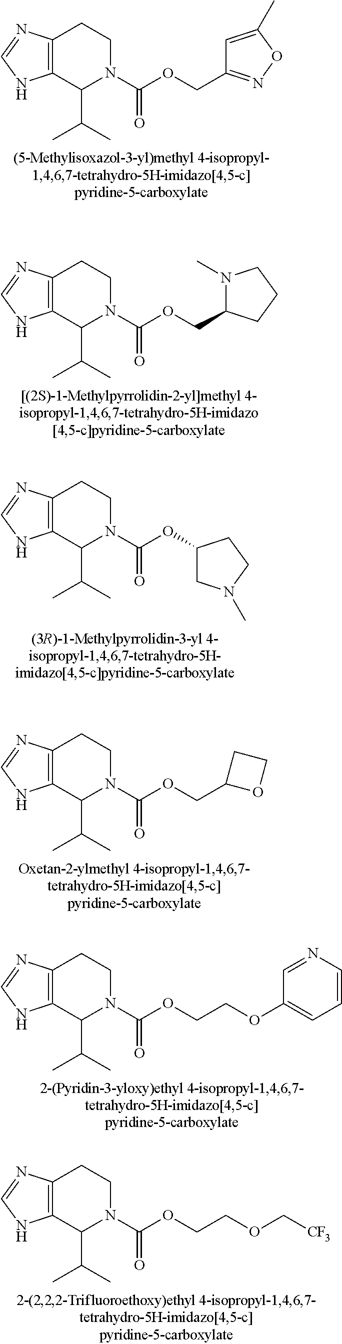 [SMILES: Cc1cc(COC(=O)N2CCc3nc[nH]c3C2C(C)C)no1 ; CC(C)C1c2[nH]cnc2CCN1C(=O)OC[C@@H]1CCCN1C ; CC(C)C1c2[nH]cnc2CCN1C(=O)O[C@@H]1CCN(C)C1 ; CC(C)C1c2[nH]cnc2CCN1C(=O)OCC1CCO1 ; CC(C)C1c2[nH]cnc2CCN1C(=O)OCCOc1cccnc1 ; CC(C)C1c2[nH]cnc2CCN1C(=O)OCCOCC(F)(F)F]

Other specific examples of inhibitors of VAP-1 include the following, which are Examples from WO2011/113798:

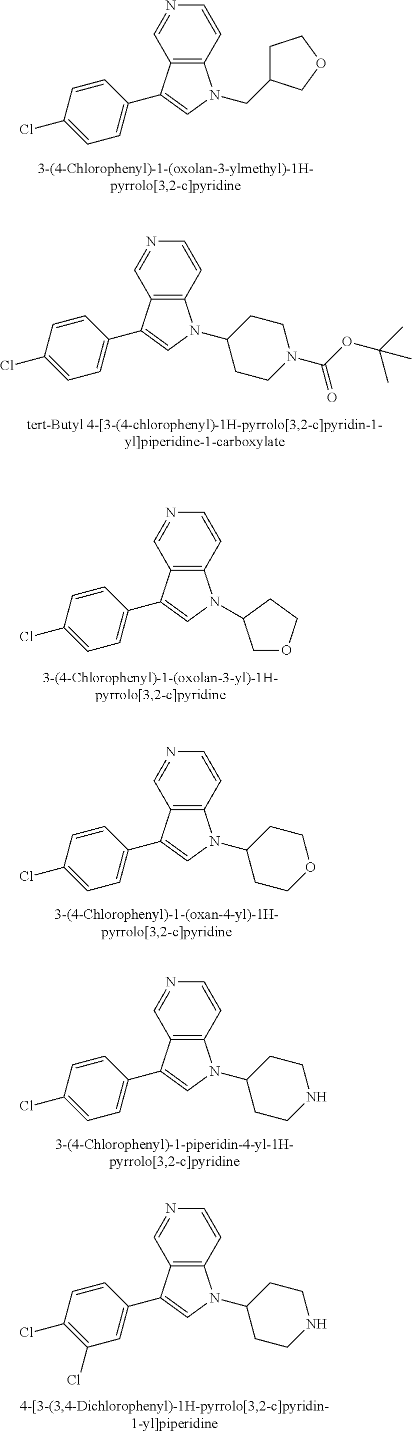 [SMILES: Clc1ccc(-c2cn(CC3CCOC3)c3ccncc23)cc1 ; CC(C)(C)OC(=O)N1CCC(n2cc(-c3ccc(Cl)cc3)c3cnccc32)CC1 ; Clc1ccc(-c2cn(C3CCOC3)c3ccncc23)cc1 ; Clc1ccc(-c2cn(C3CCOCC3)c3ccncc23)cc1 ; Clc1ccc(-c2cn(C3CCNCC3)c3ccncc23)cc1 ; Clc1ccc(-c2cn(C3CCNCC3)c3ccncc23)cc1Cl]

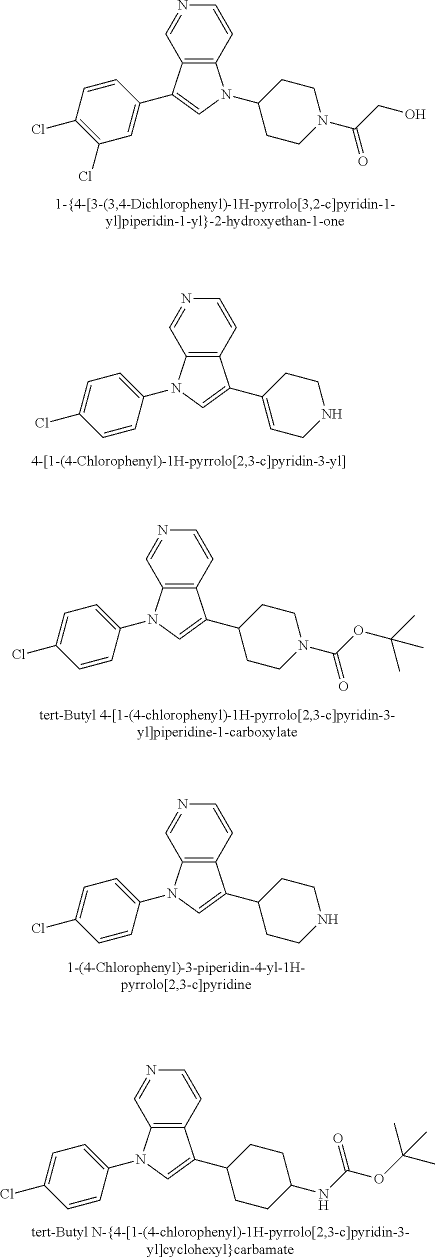 [SMILES: O=C(CO)N1CCC(n2cc(-c3ccc(Cl)c(Cl)c3)c3cnccc32)CC1 ; Clc1ccc(-n2cc(C3=CCNCC3)c3ccncc32)cc1 ; CC(C)(C)OC(=O)N1CCC(c2cn(-c3ccc(Cl)cc3)c3cnccc23)CC1 ; Clc1ccc(-n2cc(C3CCNCC3)c3ccncc32)cc1 ; CC(C)(C)OC(=O)NC1CCC(c2cn(-c3ccc(Cl)cc3)c3cnccc23)CC1]

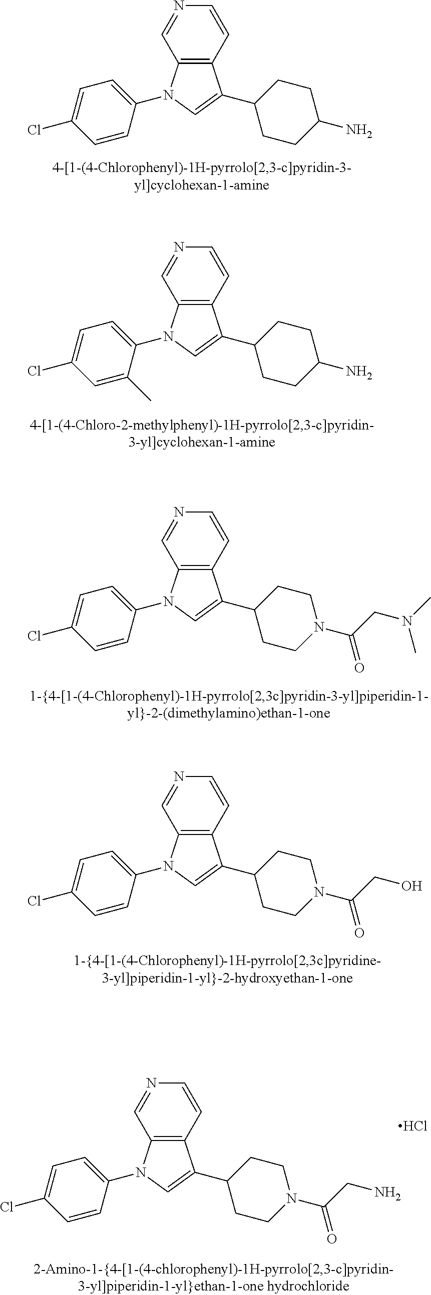 [SMILES: NC1CCC(c2cn(-c3ccc(Cl)cc3)c3cnccc23)CC1 ; Cc1cc(Cl)ccc1-n1cc(C2CCC(N)CC2)c2ccncc21 ; CN(C)CC(=O)N1CCC(c2cn(-c3ccc(Cl)cc3)c3cnccc23)CC1 ; O=C(CO)N1CCC(c2cn(-c3ccc(Cl)cc3)c3cnccc23)CC1 ; NCC(=O)N1CCC(c2cn(-c3ccc(Cl)cc3)c3cnccc23)CC1 ; Cl]

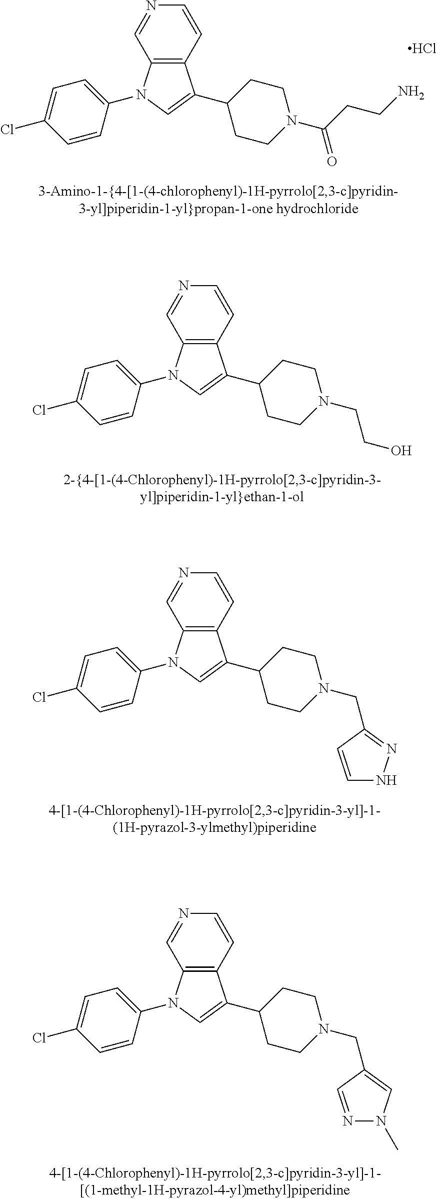 [SMILES: NCCC(=O)N1CCC(c2cn(-c3ccc(Cl)cc3)c3cnccc23)CC1 ; Cl ; OCCN1CCC(c2cn(-c3ccc(Cl)cc3)c3cnccc23)CC1 ; Clc1ccc(-n2cc(C3CCN(Cc4cc[nH]n4)CC3)c3ccncc32)cc1 ; Cn1cc(CN2CCC(c3cn(-c4ccc(Cl)cc4)c4cnccc34)CC2)cn1]

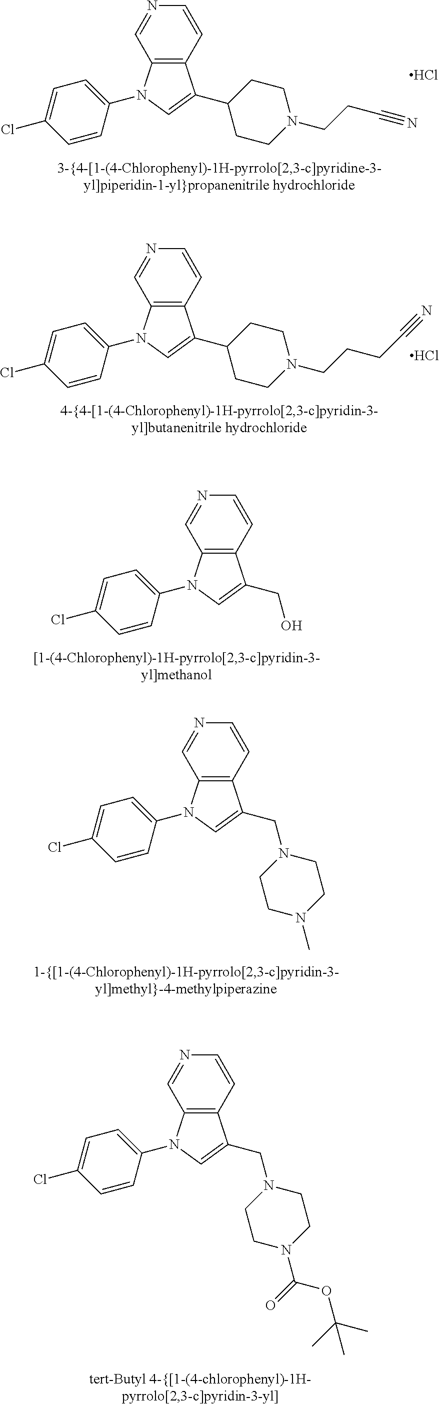

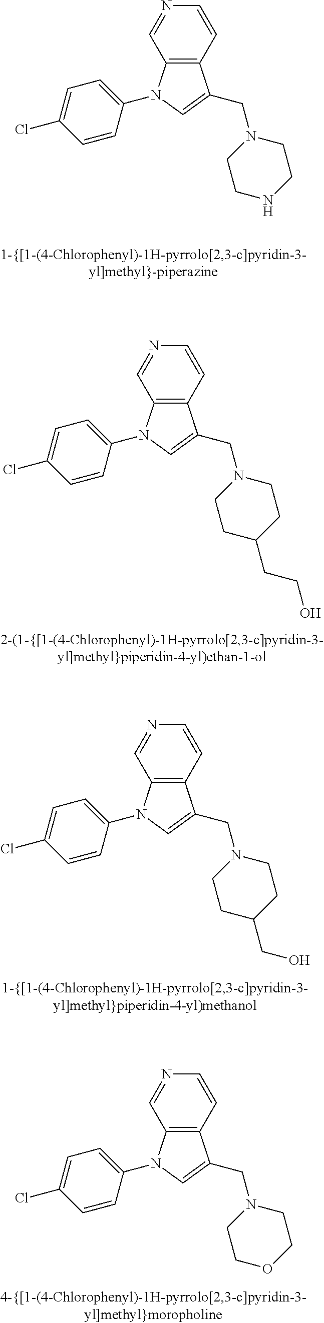 [SMILES: Clc1ccc(-n2cc(CN3CCNCC3)c3ccncc32)cc1 ; OCCC1CCN(Cc2cn(-c3ccc(Cl)cc3)c3cnccc23)CC1 ; OCC1CCN(Cc2cn(-c3ccc(Cl)cc3)c3cnccc23)CC1 ; Clc1ccc(-n2cc(CN3CCOCC3)c3ccncc32)cc1]

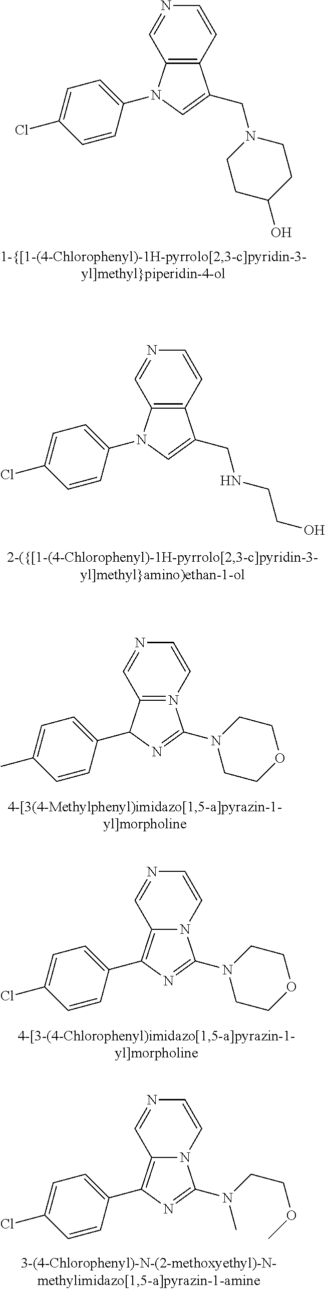

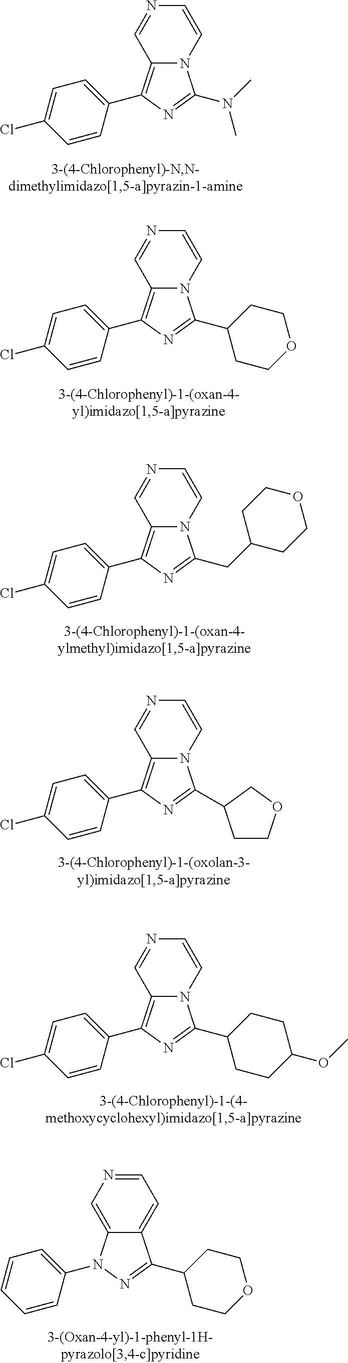 [SMILES: CN(C)c1nc(-c2ccc(Cl)cc2)c2cnccn12 ; Clc1ccc(-c2nc(C3CCOCC3)n3ccncc23)cc1 ; Clc1ccc(-c2nc(CC3CCOCC3)n3ccncc23)cc1 ; Clc1ccc(-c2nc(C3CCOC3)n3ccncc23)cc1 ; COC1CCC(c2nc(-c3ccc(Cl)cc3)c3cnccn23)CC1 ; c1ccc(-n2nc(C3CCOCC3)c3ccncc32)cc1]

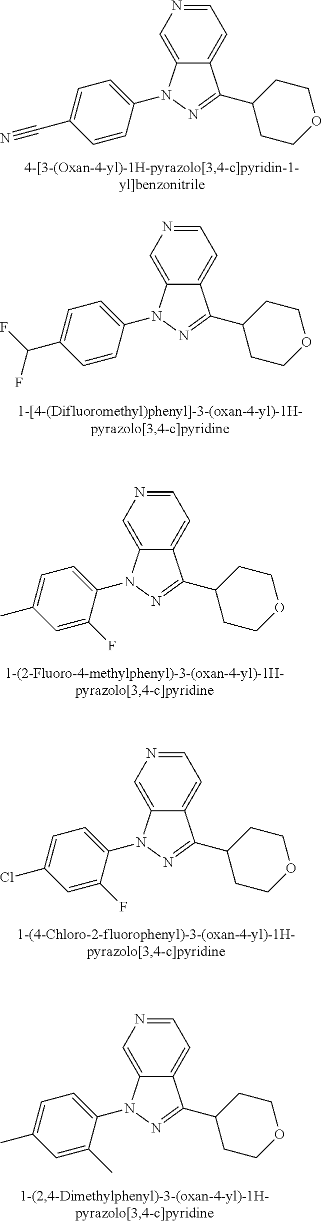 [SMILES: N#Cc1ccc(-n2nc(C3CCOCC3)c3ccncc32)cc1 ; FC(F)c1ccc(-n2nc(C3CCOCC3)c3ccncc32)cc1 ; Cc1ccc(-n2nc(C3CCOCC3)c3ccncc32)c(F)c1 ; Fc1cc(Cl)ccc1-n1nc(C2CCOCC2)c2ccncc21 ; Cc1ccc(-n2nc(C3CCOCC3)c3ccncc32)c(C)c1]

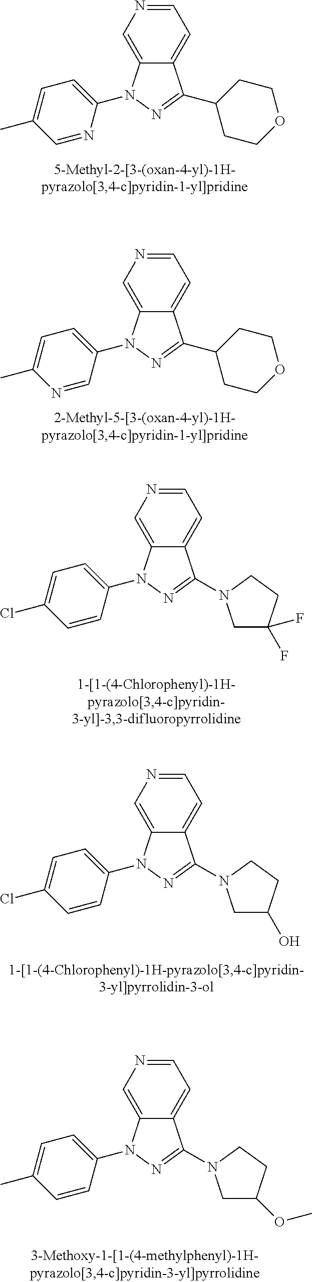

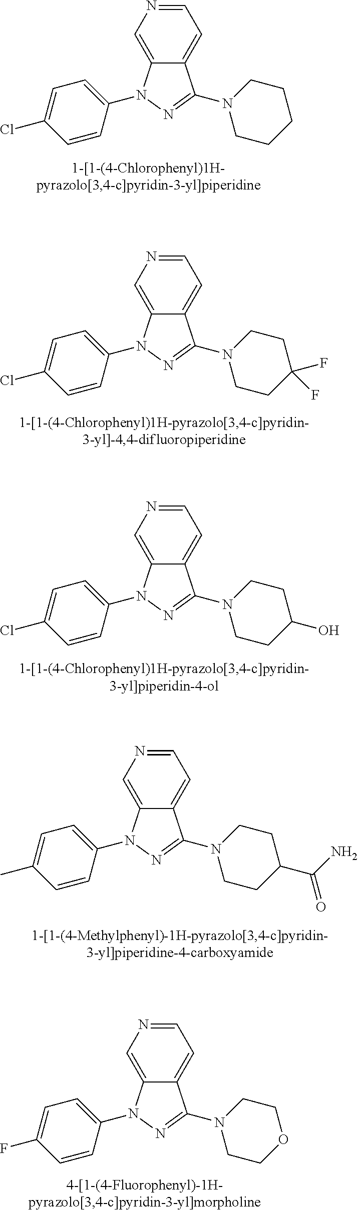 [SMILES: Clc1ccc(-n2nc(N3CCCCC3)c3ccncc32)cc1 ; FC1(F)CCN(c2nn(-c3ccc(Cl)cc3)c3cnccc23)CC1 ; OC1CCN(c2nn(-c3ccc(Cl)cc3)c3cnccc23)CC1 ; Cc1ccc(-n2nc(N3CCC(C(N)=O)CC3)c3ccncc32)cc1 ; Fc1ccc(-n2nc(N3CCOCC3)c3ccncc32)cc1]

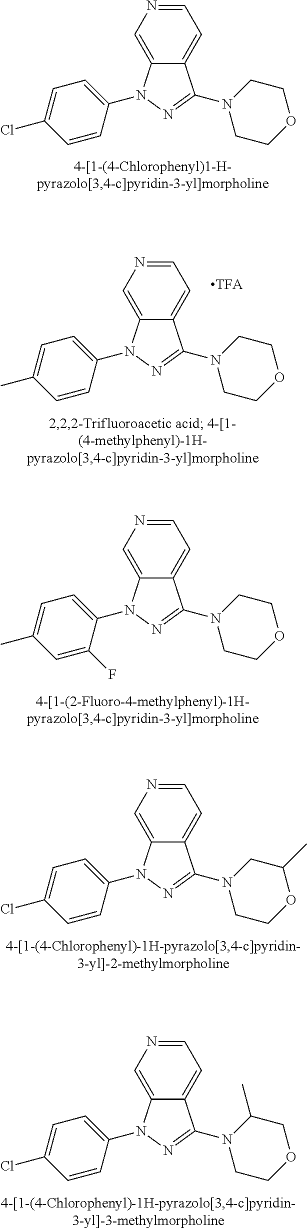 [SMILES: Clc1ccc(-n2nc(N3CCOCC3)c3ccncc32)cc1 ; Cc1ccc(-n2nc(N3CCOCC3)c3ccncc32)cc1 ; Cc1ccc(-n2nc(N3CCOCC3)c3ccncc32)c(F)c1 ; CC1CN(c2nn(-c3ccc(Cl)cc3)c3cnccc23)CCO1 ; CC1COCCN1c1nn(-c2ccc(Cl)cc2)c2cnccc12]

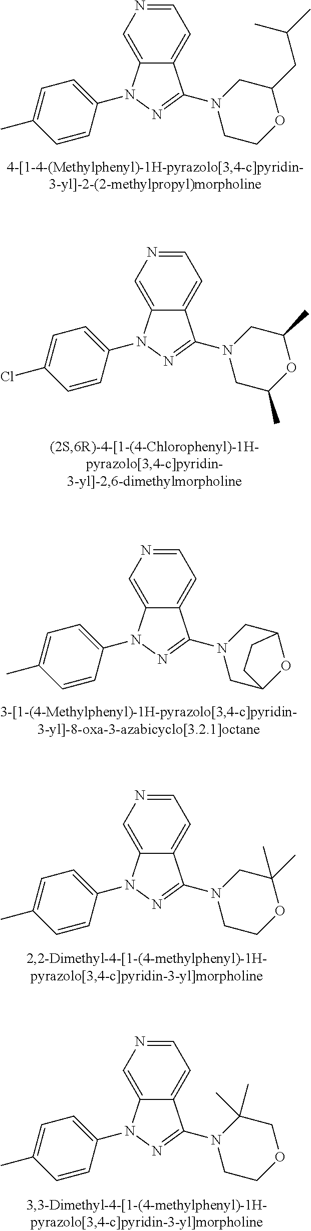

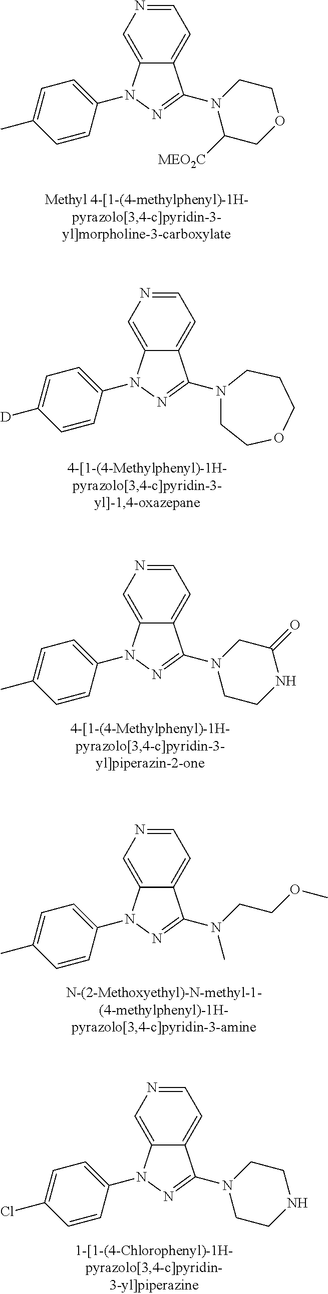

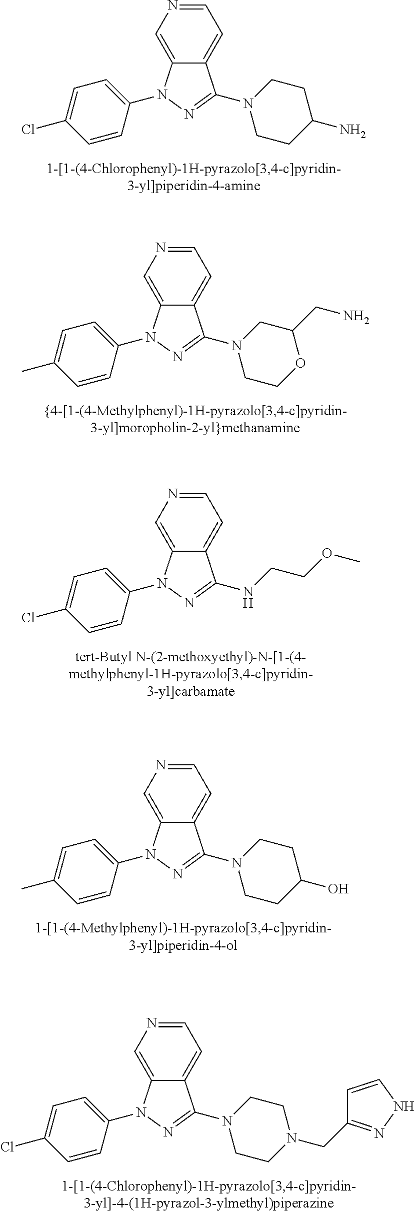

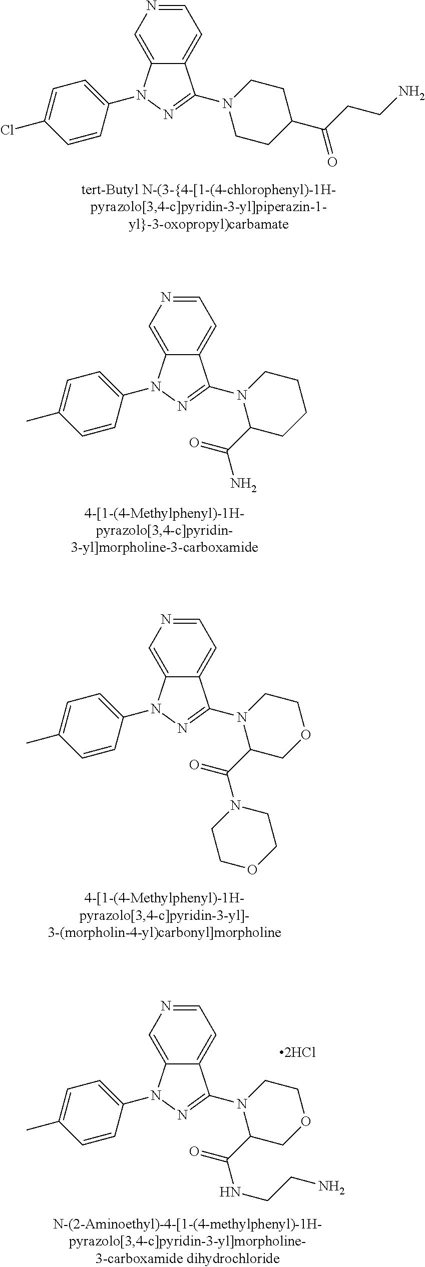 [SMILES: NCCC(=O)C1CCN(c2nn(-c3ccc(Cl)cc3)c3cnccc23)CC1 ; Cc1ccc(-n2nc(N3CCCCC3C(N)=O)c3ccncc32)cc1 ; Cc1ccc(-n2nc(N3CCOCC3C(=O)N3CCOCC3)c3ccncc32)cc1 ; Cc1ccc(-n2nc(N3CCOCC3C(=O)NCCN)c3ccncc32)cc1]

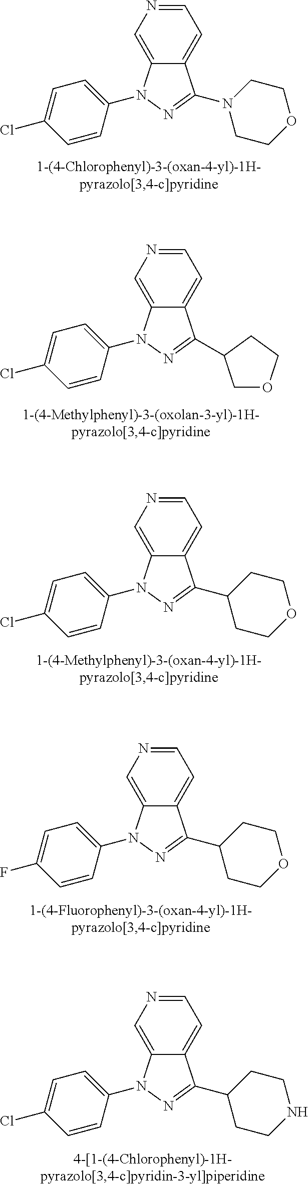 [SMILES: Clc1ccc(-n2nc(N3CCOCC3)c3ccncc32)cc1 ; Clc1ccc(-n2nc(C3CCOC3)c3ccncc32)cc1 ; Clc1ccc(-n2nc(C3CCOCC3)c3ccncc32)cc1 ; Fc1ccc(-n2nc(C3CCOCC3)c3ccncc32)cc1 ; Clc1ccc(-n2nc(C3CCNCC3)c3ccncc32)cc1]

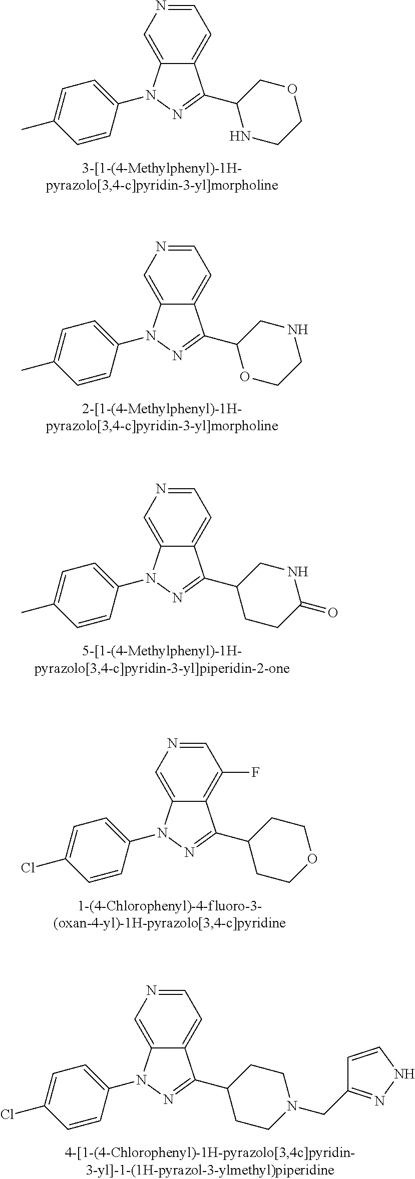 [SMILES: Cc1ccc(-n2nc(C3COCCN3)c3ccncc32)cc1 ; Cc1ccc(-n2nc(C3CNCCO3)c3ccncc32)cc1 ; Cc1ccc(-n2nc(C3CCC(=O)NC3)c3ccncc32)cc1 ; Fc1cncc2c1c(C1CCOCC1)nn2-c1ccc(Cl)cc1 ; Clc1ccc(-n2nc(C3CCN(Cc4cc[nH]n4)CC3)c3ccncc32)cc1]

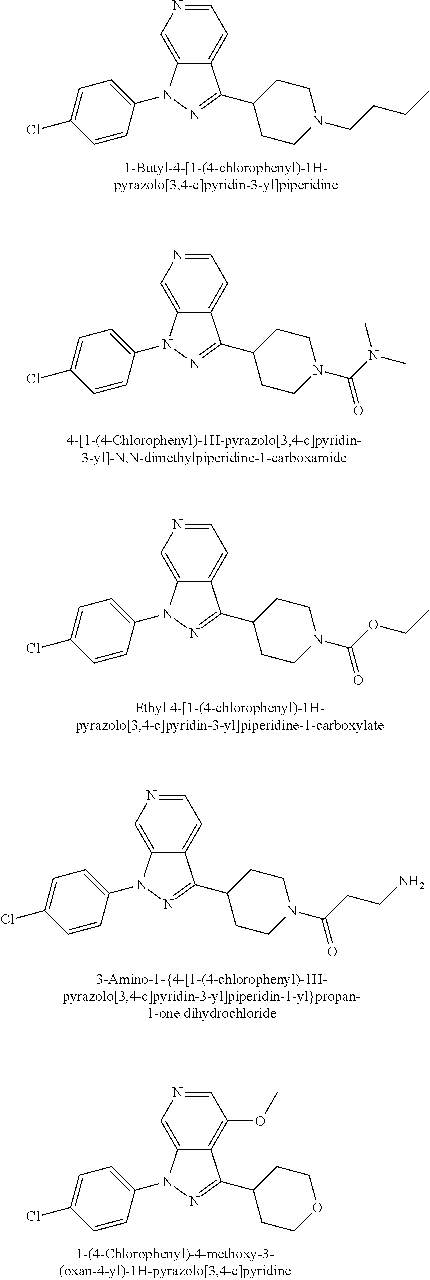 [SMILES: CCCCN1CCC(c2nn(-c3ccc(Cl)cc3)c3cnccc23)CC1 ; CN(C)C(=O)N1CCC(c2nn(-c3ccc(Cl)cc3)c3cnccc23)CC1 ; CCOC(=O)N1CCC(c2nn(-c3ccc(Cl)cc3)c3cnccc23)CC1 ; NCCC(=O)N1CCC(c2nn(-c3ccc(Cl)cc3)c3cnccc23)CC1 ; COc1cncc2c1c(C1CCOCC1)nn2-c1ccc(Cl)cc1]

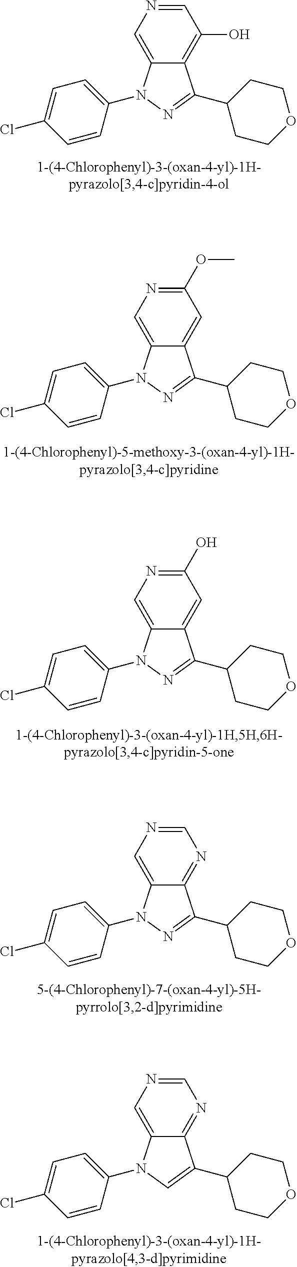 [SMILES: Oc1cncc2c1c(C1CCOCC1)nn2-c1ccc(Cl)cc1 ; COc1cc2c(C3CCOCC3)nn(-c3ccc(Cl)cc3)c2cn1 ; Oc1cc2c(C3CCOCC3)nn(-c3ccc(Cl)cc3)c2cn1 ; Clc1ccc(-n2nc(C3CCOCC3)c3ncncc32)cc1 ; Clc1ccc(-n2cc(C3CCOCC3)c3ncncc32)cc1]

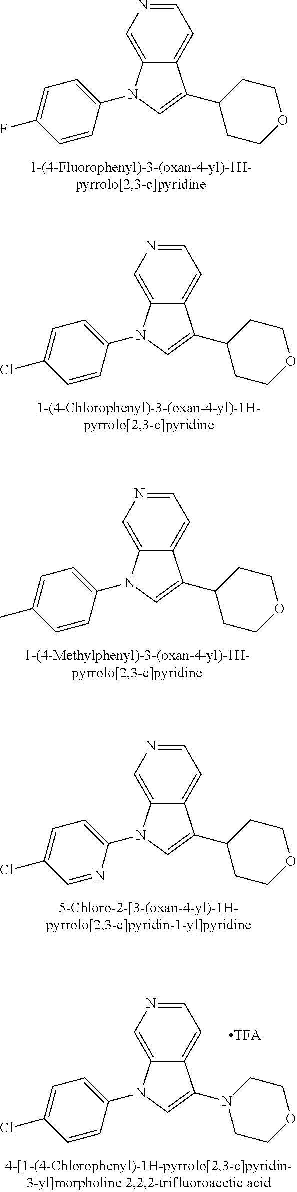 [SMILES: Fc1ccc(-n2cc(C3CCOCC3)c3ccncc32)cc1 ; Clc1ccc(-n2cc(C3CCOCC3)c3ccncc32)cc1 ; Cc1ccc(-n2cc(C3CCOCC3)c3ccncc32)cc1 ; Clc1ccc(-n2cc(C3CCOCC3)c3ccncc32)nc1 ; Clc1ccc(-n2cc(N3CCOCC3)c3ccncc32)cc1]

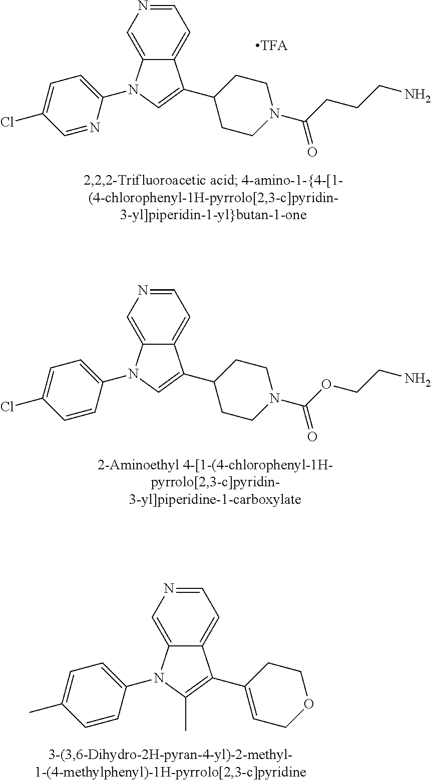 [SMILES: NCCCC(=O)N1CCC(c2cn(-c3ccc(Cl)cn3)c3cnccc23)CC1 ; NCCOC(=O)N1CCC(c2cn(-c3ccc(Cl)cc3)c3cnccc23)CC1 ; Cc1ccc(-n2c(C)c(C3=CCOCC3)c3ccncc32)cc1]

Further specific VAP-1 compounds include the Examples of WO2013/037411, namely:

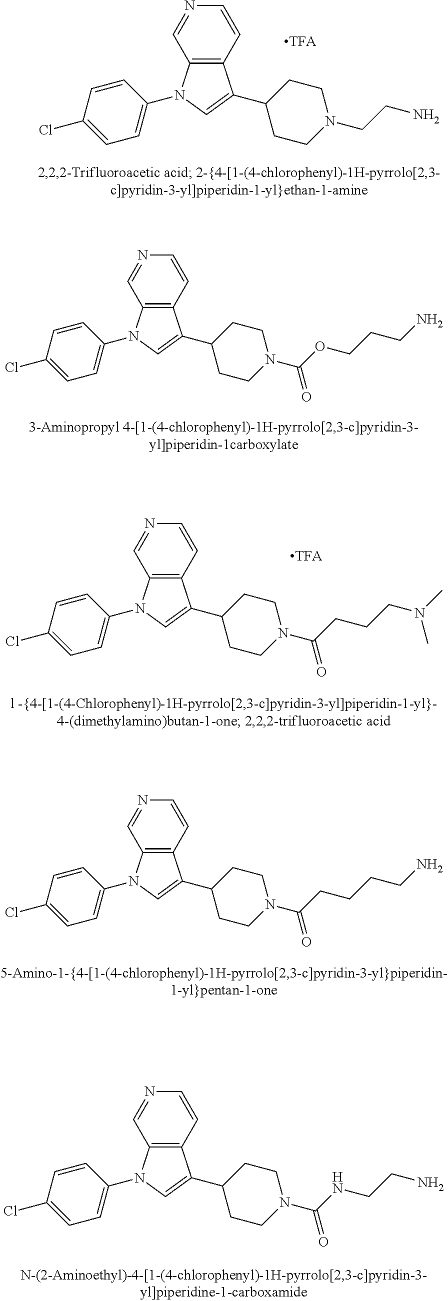 [SMILES: NCCN1CCC(c2cn(-c3ccc(Cl)cc3)c3cnccc23)CC1 ; NCCCOC(=O)N1CCC(c2cn(-c3ccc(Cl)cc3)c3cnccc23)CC1 ; CN(C)CCCC(=O)N1CCC(c2cn(-c3ccc(Cl)cc3)c3cnccc23)CC1 ; NCCCCC(=O)N1CCC(c2cn(-c3ccc(Cl)cc3)c3cnccc23)CC1 ; NCCNC(=O)N1CCC(c2cn(-c3ccc(Cl)cc3)c3cnccc23)CC1]

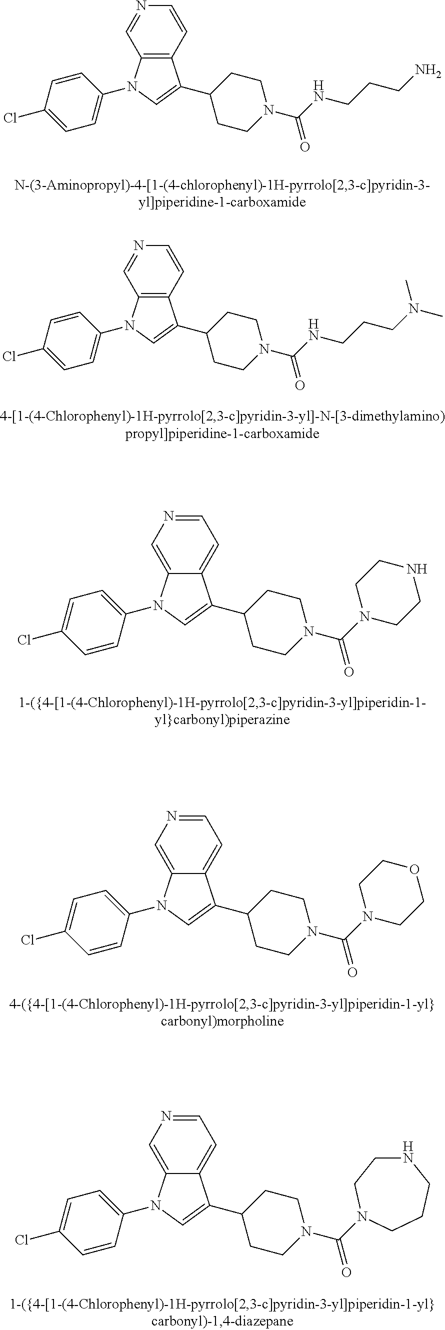 [SMILES: NCCCNC(=O)N1CCC(c2cn(-c3ccc(Cl)cc3)c3cnccc23)CC1 ; CN(C)CCCNC(=O)N1CCC(c2cn(-c3ccc(Cl)cc3)c3cnccc23)CC1 ; O=C(N1CCNCC1)N1CCC(c2cn(-c3ccc(Cl)cc3)c3cnccc23)CC1 ; O=C(N1CCOCC1)N1CCC(c2cn(-c3ccc(Cl)cc3)c3cnccc23)CC1 ; O=C(N1CCCNCC1)N1CCC(c2cn(-c3ccc(Cl)cc3)c3cnccc23)CC1]

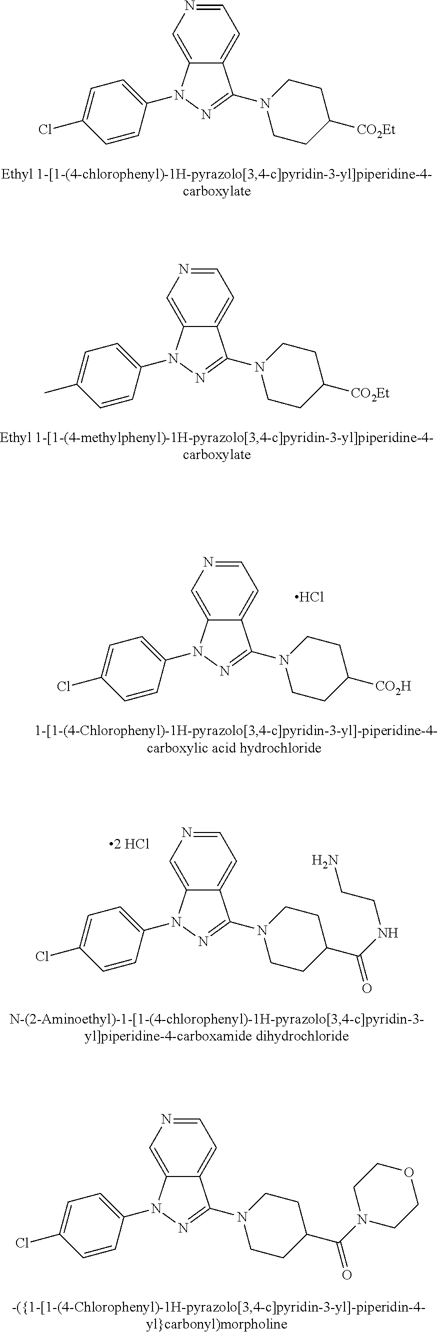 [SMILES: CCOC(=O)C1CCN(c2nn(-c3ccc(Cl)cc3)c3cnccc23)CC1 ; CCOC(=O)C1CCN(c2nn(-c3ccc(C)cc3)c3cnccc23)CC1 ; O=C(O)C1CCN(c2nn(-c3ccc(Cl)cc3)c3cnccc23)CC1 ; Cl ; NCCNC(=O)C1CCN(c2nn(-c3ccc(Cl)cc3)c3cnccc23)CC1 ; O=C(C1CCN(c2nn(-c3ccc(Cl)cc3)c3cnccc23)CC1)N1CCOCC1]

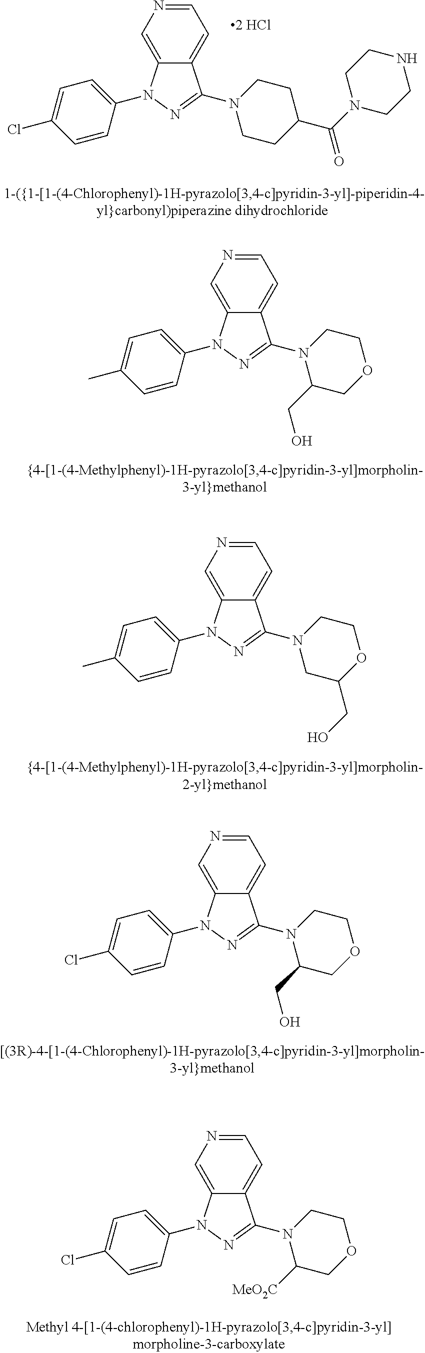 [SMILES: O=C(C1CCN(c2nn(-c3ccc(Cl)cc3)c3cnccc23)CC1)N1CCNCC1 ; Cc1ccc(-n2nc(N3CCOCC3CO)c3ccncc32)cc1 ; Cc1ccc(-n2nc(N3CCOC(CO)C3)c3ccncc32)cc1 ; OC[C@@H]1COCCN1c1nn(-c2ccc(Cl)cc2)c2cnccc12 ; COC(=O)C1COCCN1c1nn(-c2ccc(Cl)cc2)c2cnccc12]

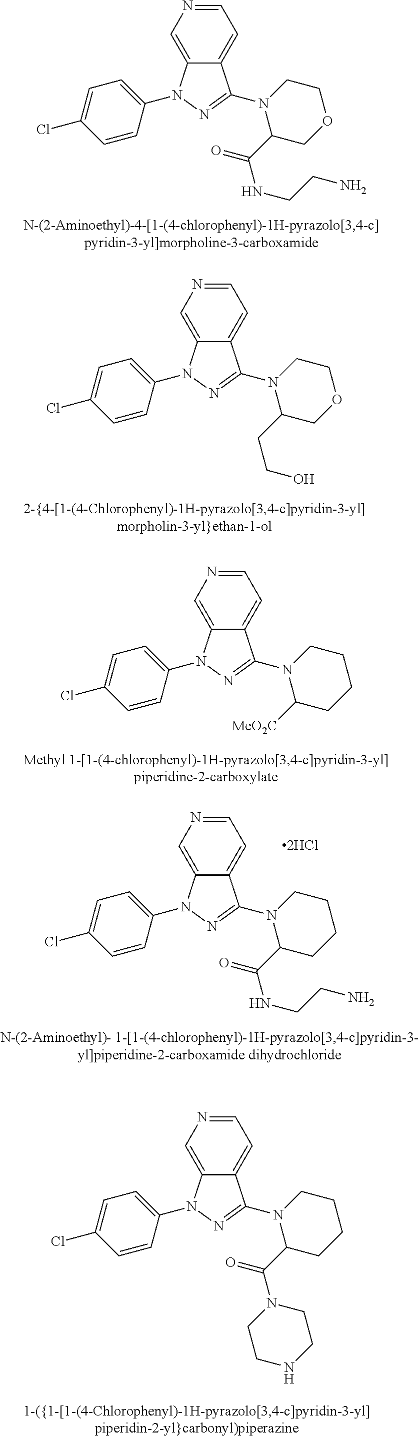 [SMILES: NCCNC(=O)C1COCCN1c1nn(-c2ccc(Cl)cc2)c2cnccc12 ; OCCC1COCCN1c1nn(-c2ccc(Cl)cc2)c2cnccc12 ; COC(=O)C1CCCCN1c1nn(-c2ccc(Cl)cc2)c2cnccc12 ; NCCNC(=O)C1CCCCN1c1nn(-c2ccc(Cl)cc2)c2cnccc12 ; O=C(C1CCCCN1c1nn(-c2ccc(Cl)cc2)c2cnccc12)N1CCNCC1]

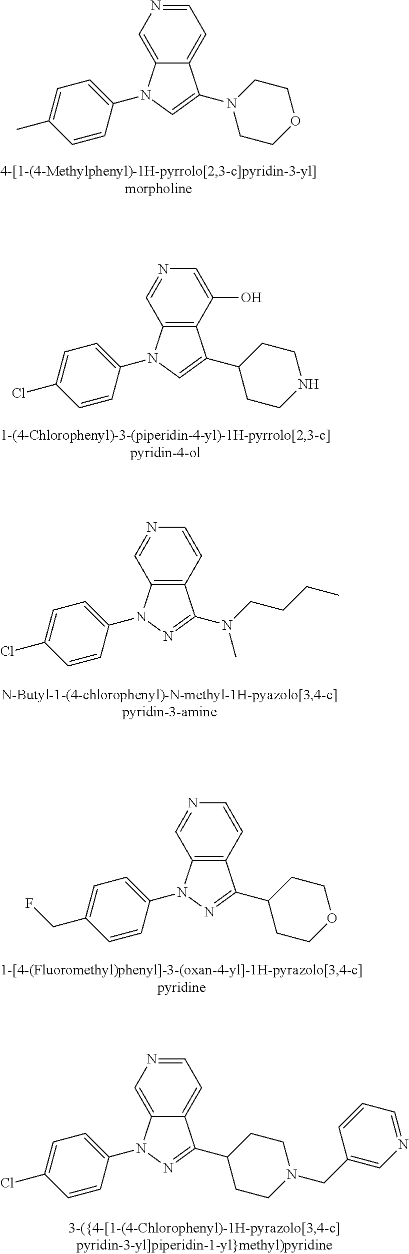 [SMILES: Cc1ccc(-n2cc(N3CCOCC3)c3ccncc32)cc1 ; Oc1cncc2c1c(C1CCNCC1)cn2-c1ccc(Cl)cc1 ; CCCCN(C)c1nn(-c2ccc(Cl)cc2)c2cnccc12 ; FCc1ccc(-n2nc(C3CCOCC3)c3ccncc32)cc1 ; Clc1ccc(-n2nc(C3CCN(Cc4cccnc4)CC3)c3ccncc32)cc1]

Further specific examples of VAP-1 compounds include the Examples of WO2013/038189, namely:

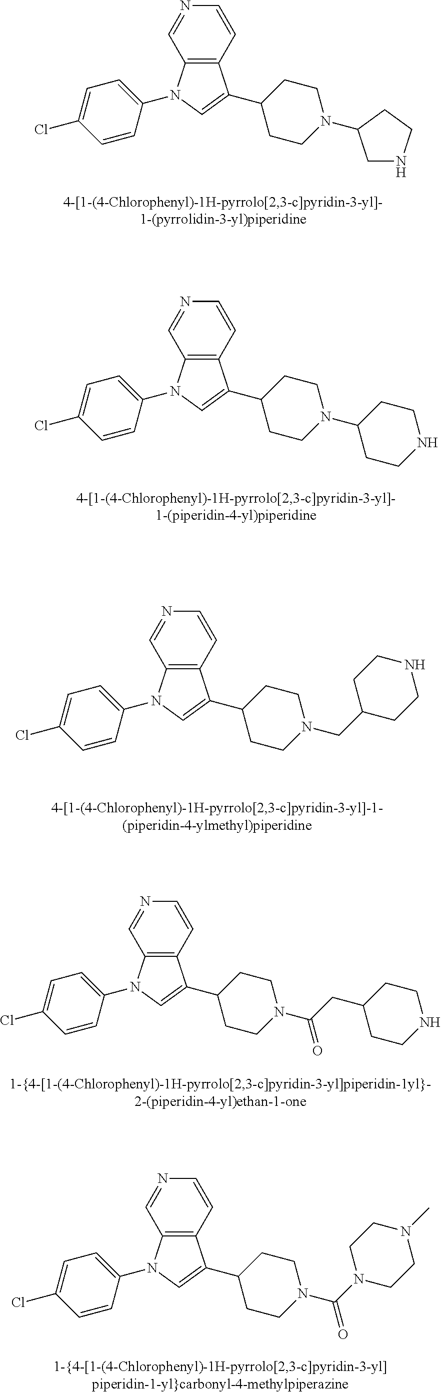 [SMILES: Clc1ccc(-n2cc(C3CCN(C4CCNC4)CC3)c3ccncc32)cc1 ; Clc1ccc(-n2cc(C3CCN(C4CCNCC4)CC3)c3ccncc32)cc1 ; Clc1ccc(-n2cc(C3CCN(CC4CCNCC4)CC3)c3ccncc32)cc1 ; O=C(CC1CCNCC1)N1CCC(c2cn(-c3ccc(Cl)cc3)c3cnccc23)CC1 ; CN1CCN(C(=O)N2CCC(c3cn(-c4ccc(Cl)cc4)c4cnccc34)CC2)CC1]

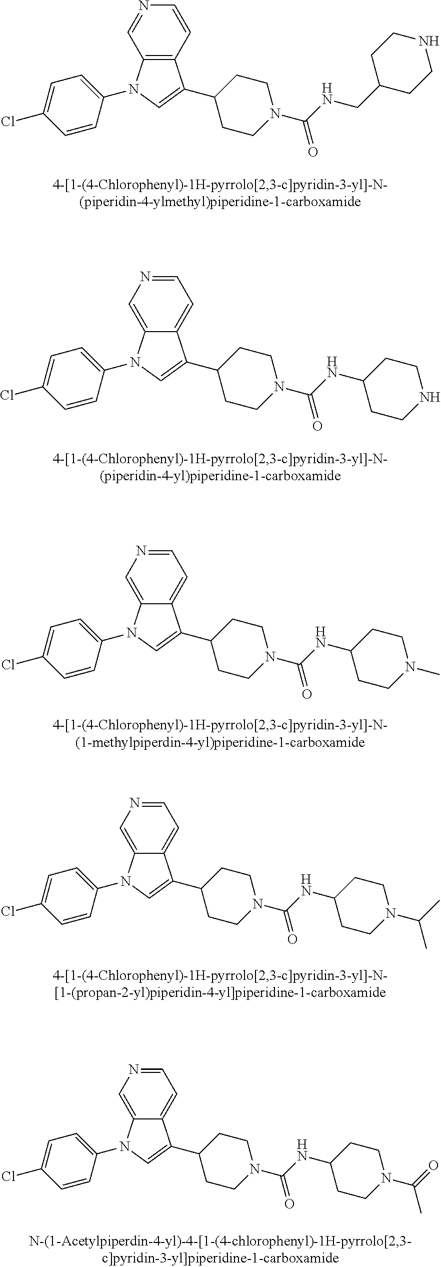 [SMILES: O=C(NCC1CCNCC1)N1CCC(c2cn(-c3ccc(Cl)cc3)c3cnccc23)CC1 ; O=C(NC1CCNCC1)N1CCC(c2cn(-c3ccc(Cl)cc3)c3cnccc23)CC1 ; CN1CCC(NC(=O)N2CCC(c3cn(-c4ccc(Cl)cc4)c4cnccc34)CC2)CC1 ; CC(C)N1CCC(NC(=O)N2CCC(c3cn(-c4ccc(Cl)cc4)c4cnccc34)CC2)CC1 ; CC(=O)N1CCC(NC(=O)N2CCC(c3cn(-c4ccc(Cl)cc4)c4cnccc34)CC2)CC1]

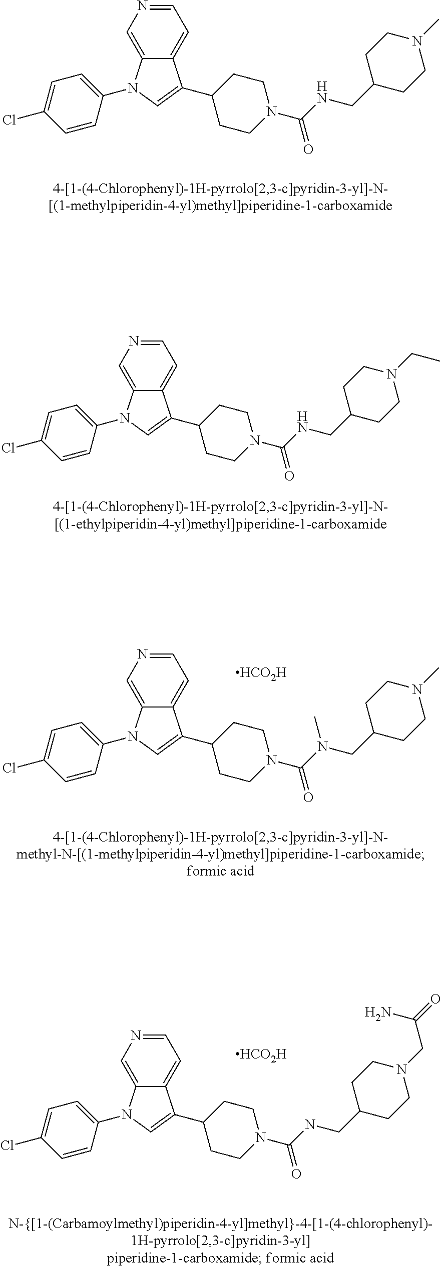 [SMILES: CN1CCC(CNC(=O)N2CCC(c3cn(-c4ccc(Cl)cc4)c4cnccc34)CC2)CC1 ; CCN1CCC(CNC(=O)N2CCC(c3cn(-c4ccc(Cl)cc4)c4cnccc34)CC2)CC1 ; CN1CCC(CN(C)C(=O)N2CCC(c3cn(-c4ccc(Cl)cc4)c4cnccc34)CC2)CC1 ; "·HCO2H" ; NC(=O)CN1CCC(CNC(=O)N2CCC(c3cn(-c4ccc(Cl)cc4)c4cnccc34)CC2)CC1 ; "·HCO2H"]

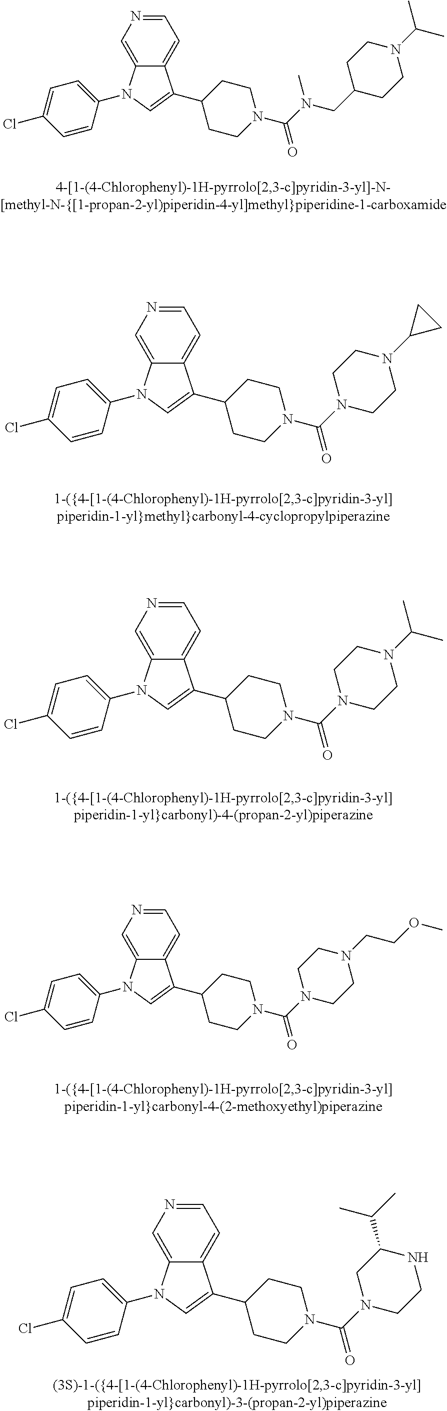 [SMILES: CC(C)N1CCC(CN(C)C(=O)N2CCC(c3cn(-c4ccc(Cl)cc4)c4cnccc34)CC2)CC1 ; O=C(N1CCC(c2cn(-c3ccc(Cl)cc3)c3cnccc23)CC1)N1CCN(C2CC2)CC1 ; CC(C)N1CCN(C(=O)N2CCC(c3cn(-c4ccc(Cl)cc4)c4cnccc34)CC2)CC1 ; COCCN1CCN(C(=O)N2CCC(c3cn(-c4ccc(Cl)cc4)c4cnccc34)CC2)CC1 ; CC(C)[C@H]1CN(C(=O)N2CCC(c3cn(-c4ccc(Cl)cc4)c4cnccc34)CC2)CCN1]

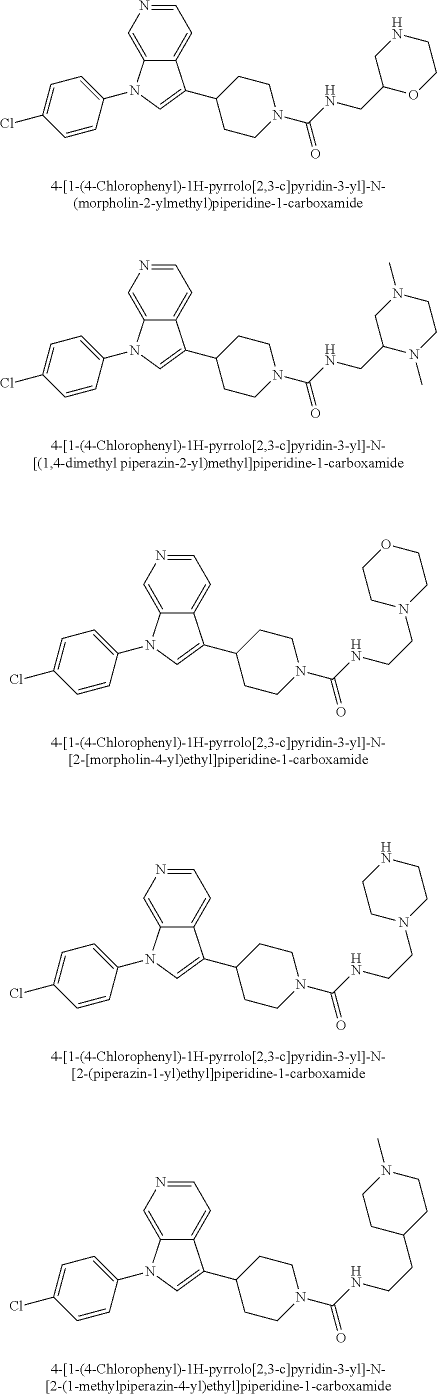 [SMILES: O=C(NCC1CNCCO1)N1CCC(c2cn(-c3ccc(Cl)cc3)c3cnccc23)CC1 ; CN1CCN(C)C(CNC(=O)N2CCC(c3cn(-c4ccc(Cl)cc4)c4cnccc34)CC2)C1 ; O=C(NCCN1CCOCC1)N1CCC(c2cn(-c3ccc(Cl)cc3)c3cnccc23)CC1 ; O=C(NCCN1CCNCC1)N1CCC(c2cn(-c3ccc(Cl)cc3)c3cnccc23)CC1 ; CN1CCC(CCNC(=O)N2CCC(c3cn(-c4ccc(Cl)cc4)c4cnccc34)CC2)CC1]

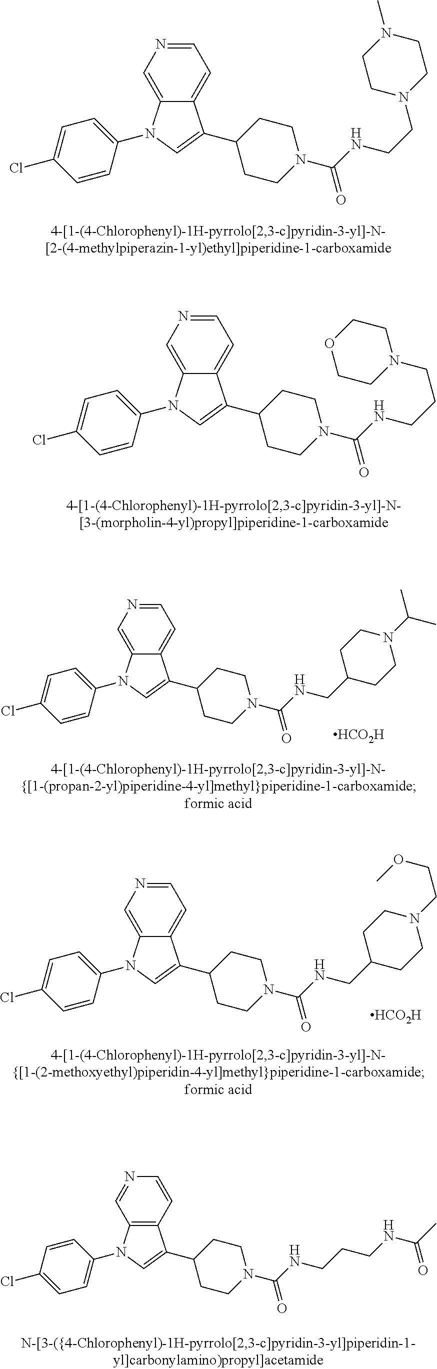

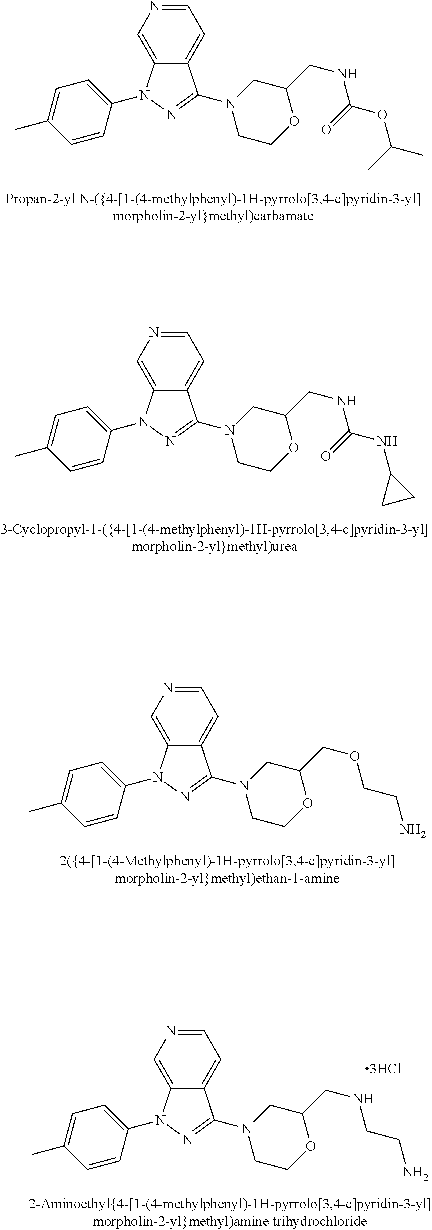

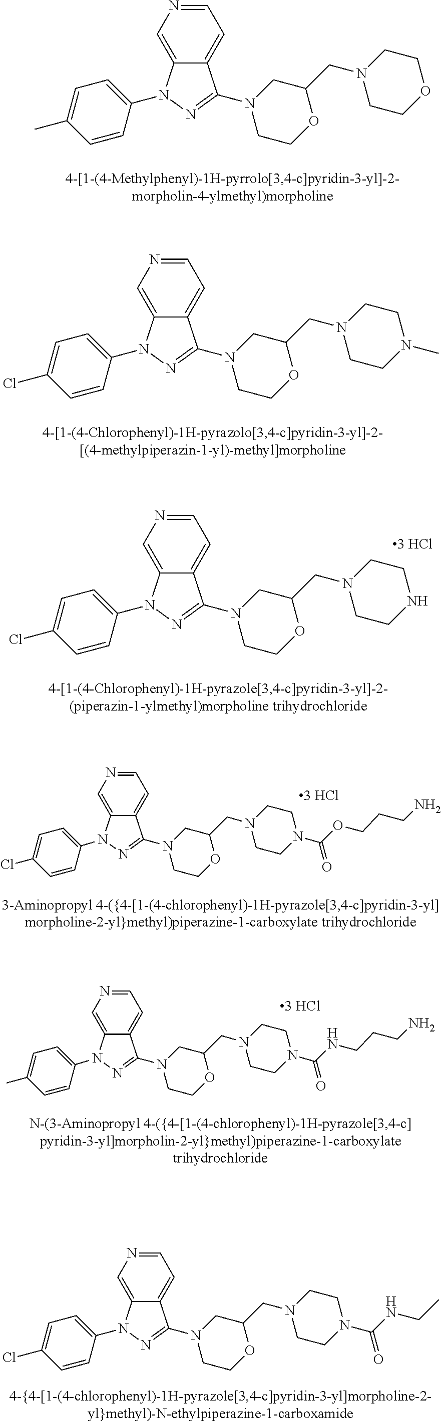

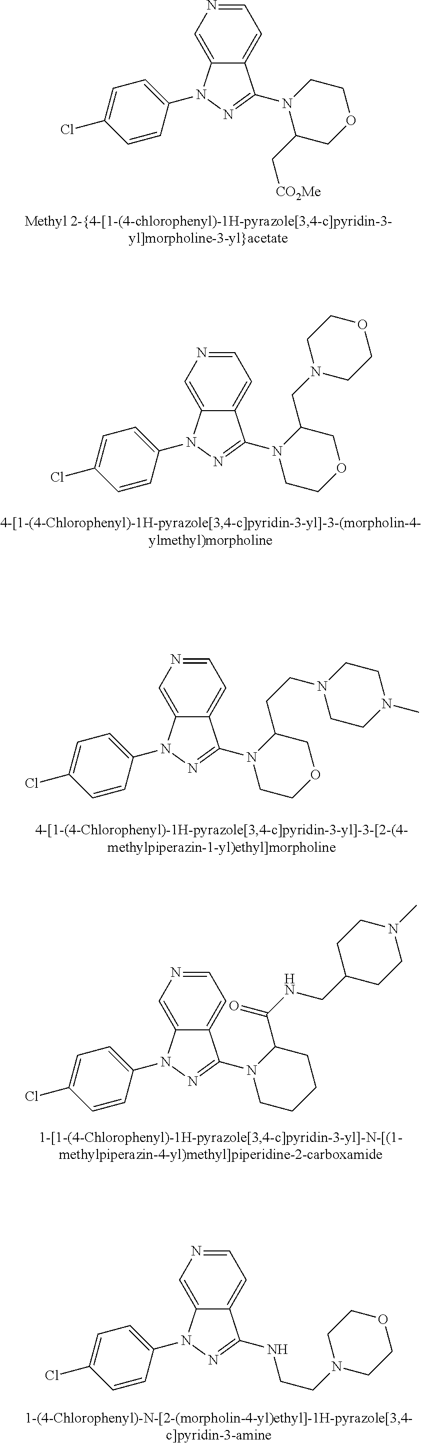 [SMILES: COC(=O)CC1COCCN1c1nn(-c2ccc(Cl)cc2)c2cnccc12 ; Clc1ccc(-n2nc(N3CCOCC3CN3CCOCC3)c3ccncc32)cc1 ; CN1CCN(CCC2COCCN2c2nn(-c3ccc(Cl)cc3)c3cnccc23)CC1 ; CN1CCC(CNC(=O)C2CCCCN2c2nn(-c3ccc(Cl)cc3)c3cnccc23)CC1 ; Clc1ccc(-n2nc(NCCN3CCOCC3)c3ccncc32)cc1]

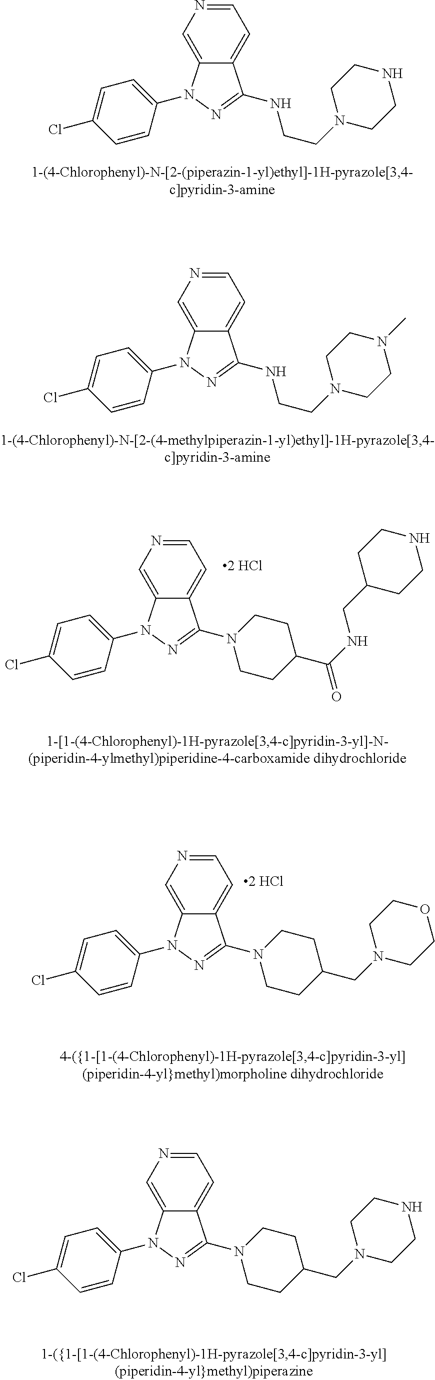 [SMILES: Clc1ccc(-n2nc(NCCN3CCNCC3)c3ccncc32)cc1 ; CN1CCN(CCNc2nn(-c3ccc(Cl)cc3)c3cnccc23)CC1 ; O=C(NCC1CCNCC1)C1CCN(c2nn(-c3ccc(Cl)cc3)c3cnccc23)CC1 ; Clc1ccc(-n2nc(N3CCC(CN4CCOCC4)CC3)c3ccncc32)cc1 ; Clc1ccc(-n2nc(N3CCC(CN4CCNCC4)CC3)c3ccncc32)cc1]

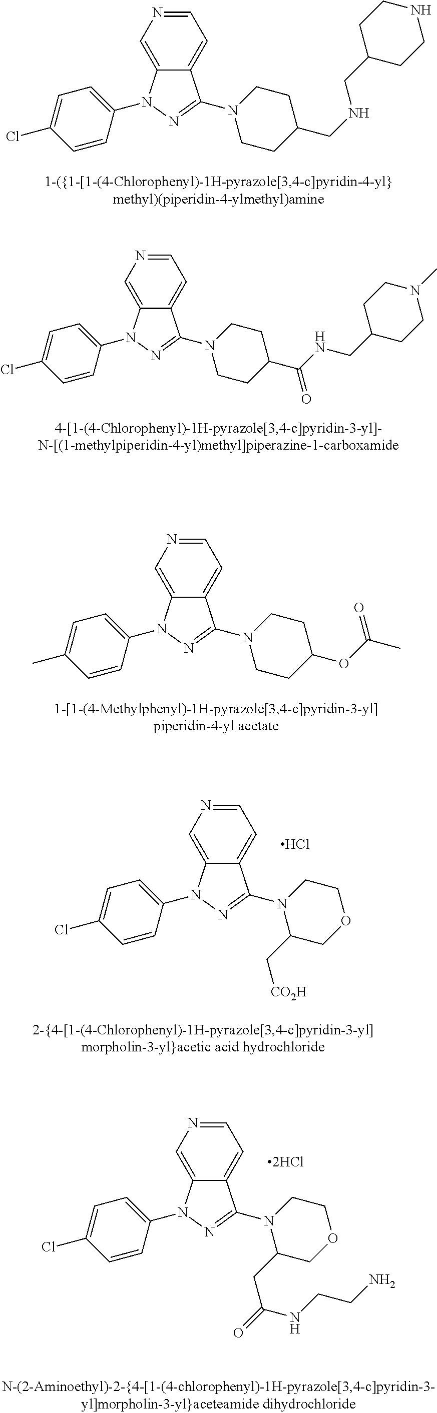

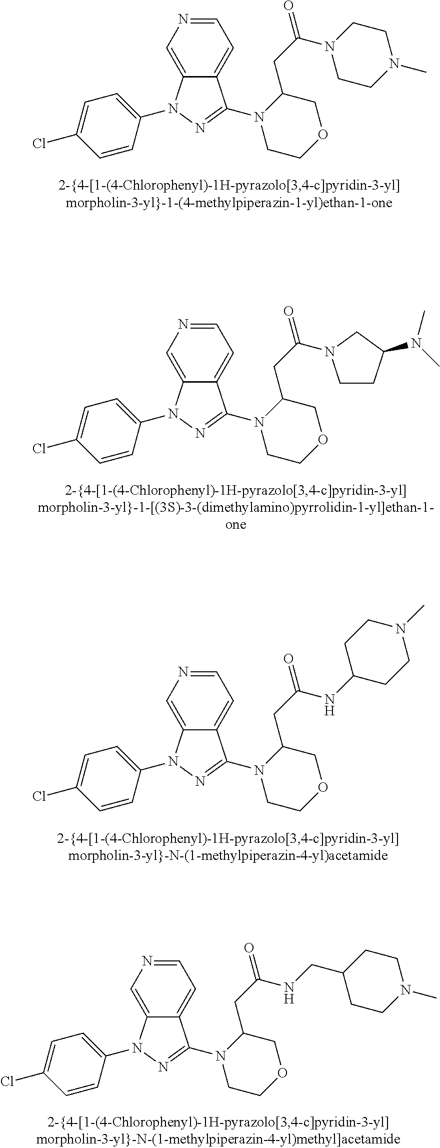 [SMILES: CN1CCN(C(=O)CC2COCCN2c2nn(-c3ccc(Cl)cc3)c3cnccc23)CC1 ; CN(C)[C@H]1CCN(C(=O)CC2COCCN2c2nn(-c3ccc(Cl)cc3)c3cnccc23)C1 ; CN1CCC(NC(=O)CC2COCCN2c2nn(-c3ccc(Cl)cc3)c3cnccc23)CC1 ; CN1CCC(CNC(=O)CC2COCCN2c2nn(-c3ccc(Cl)cc3)c3cnccc23)CC1]

Specific examples of inhibitors of VAP-1 include the compounds specifically disclosed as Examples in WO 2010/031791, namely:

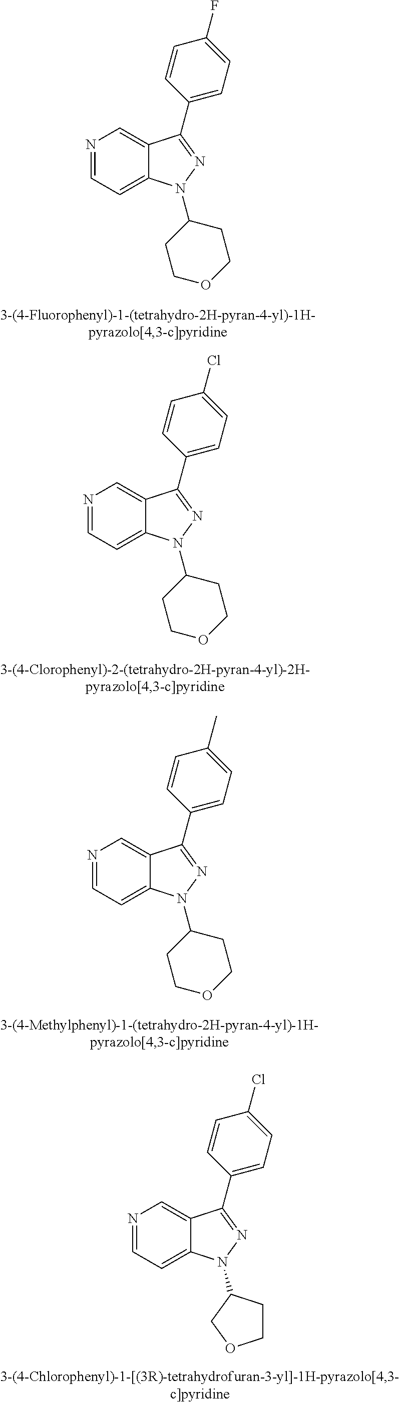

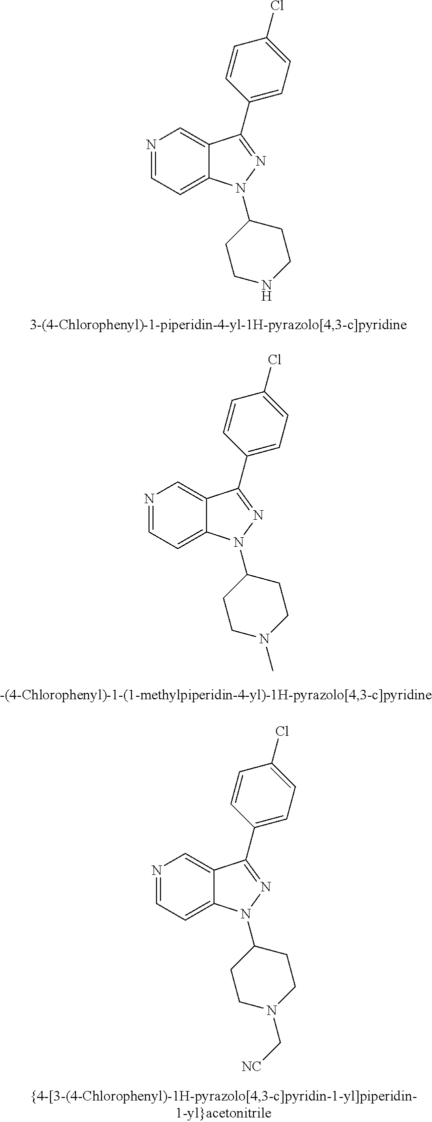 [SMILES: Clc1ccc(-c2nn(C3CCNCC3)c3ccncc23)cc1 ; CN1CCC(n2nc(-c3ccc(Cl)cc3)c3cnccc32)CC1 ; N#CCN1CCC(n2nc(-c3ccc(Cl)cc3)c3cnccc32)CC1]

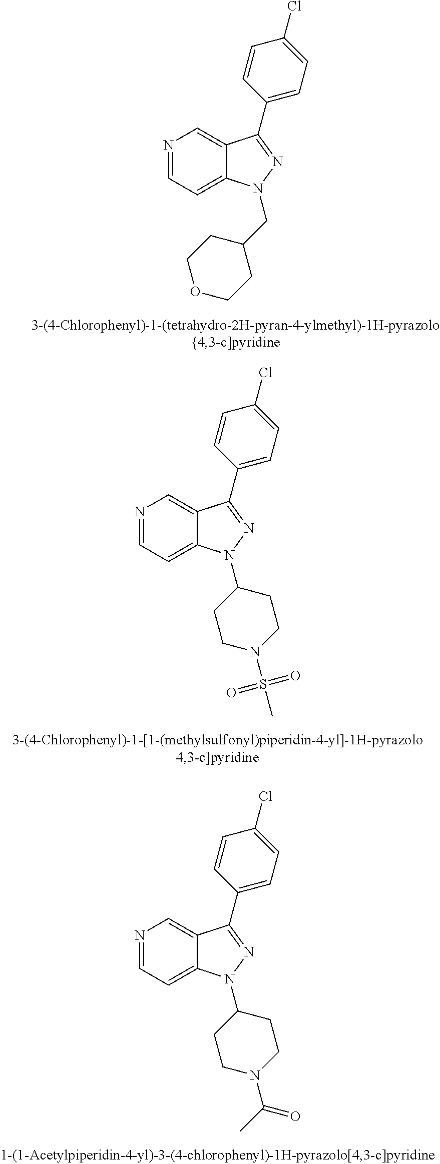 [SMILES: Clc1ccc(-c2nn(CC3CCOCC3)c3ccncc23)cc1 ; CS(=O)(=O)N1CCC(n2nc(-c3ccc(Cl)cc3)c3cnccc32)CC1 ; CC(=O)N1CCC(n2nc(-c3ccc(Cl)cc3)c3cnccc32)CC1]

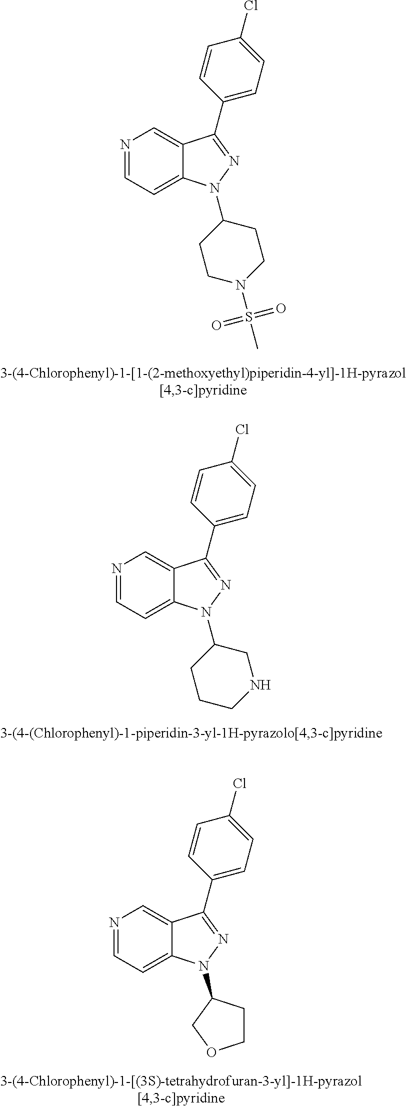 [SMILES: CS(=O)(=O)N1CCC(n2nc(-c3ccc(Cl)cc3)c3cnccc32)CC1 ; Clc1ccc(-c2nn(C3CCCNC3)c3ccncc23)cc1 ; Clc1ccc(-c2nn([C@H]3CCOC3)c3ccncc23)cc1]

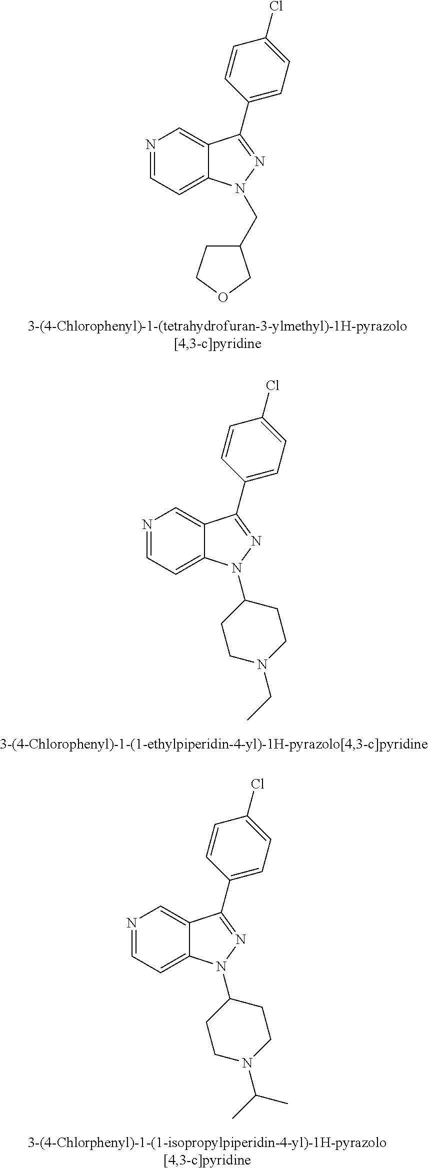 [SMILES: Clc1ccc(-c2nn(CC3CCOC3)c3ccncc23)cc1 ; CCN1CCC(n2nc(-c3ccc(Cl)cc3)c3cnccc32)CC1 ; CC(C)N1CCC(n2nc(-c3ccc(Cl)cc3)c3cnccc32)CC1]

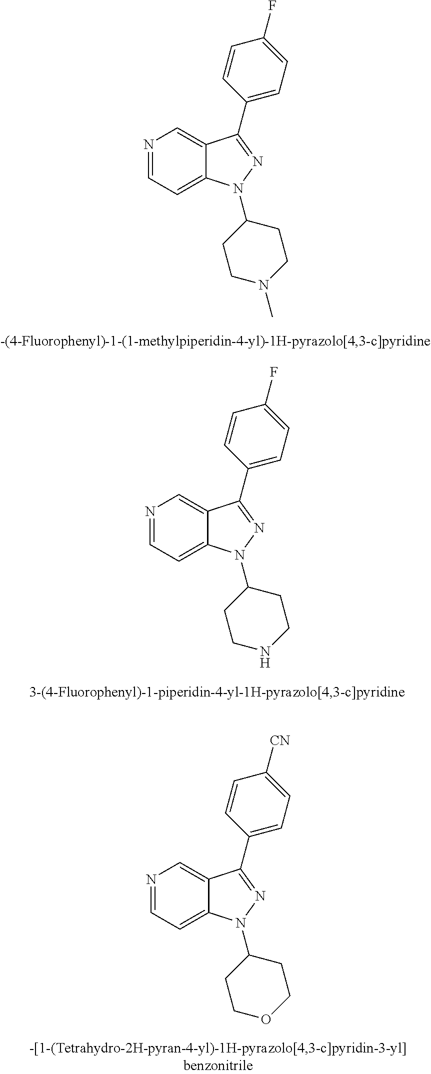 [SMILES: CN1CCC(n2nc(-c3ccc(F)cc3)c3cnccc32)CC1 ; Fc1ccc(-c2nn(C3CCNCC3)c3ccncc23)cc1 ; N#Cc1ccc(-c2nn(C3CCOCC3)c3ccncc23)cc1]

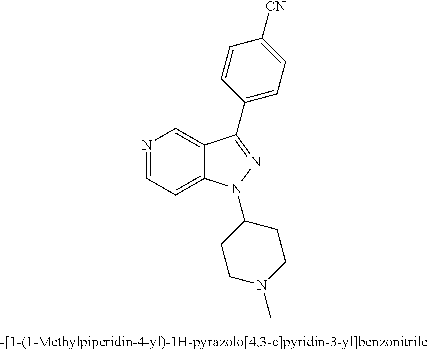 [SMILES: CN1CCC(n2nc(-c3ccc(C#N)cc3)c3cnccc32)CC1]

Specific examples of inhibitors of VAP-1 include the compounds specifically disclosed as Examples in WO 2010/064020, namely:

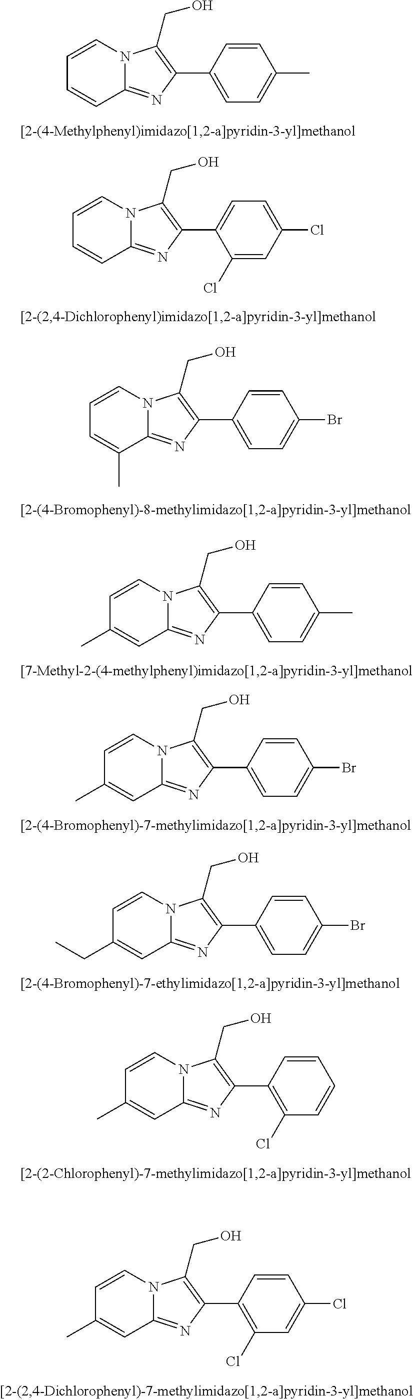

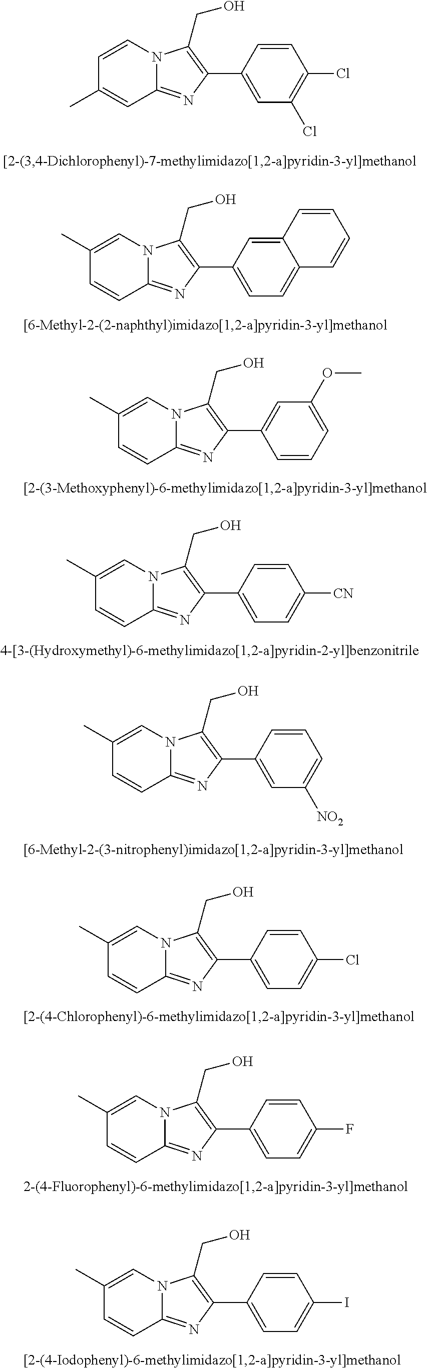

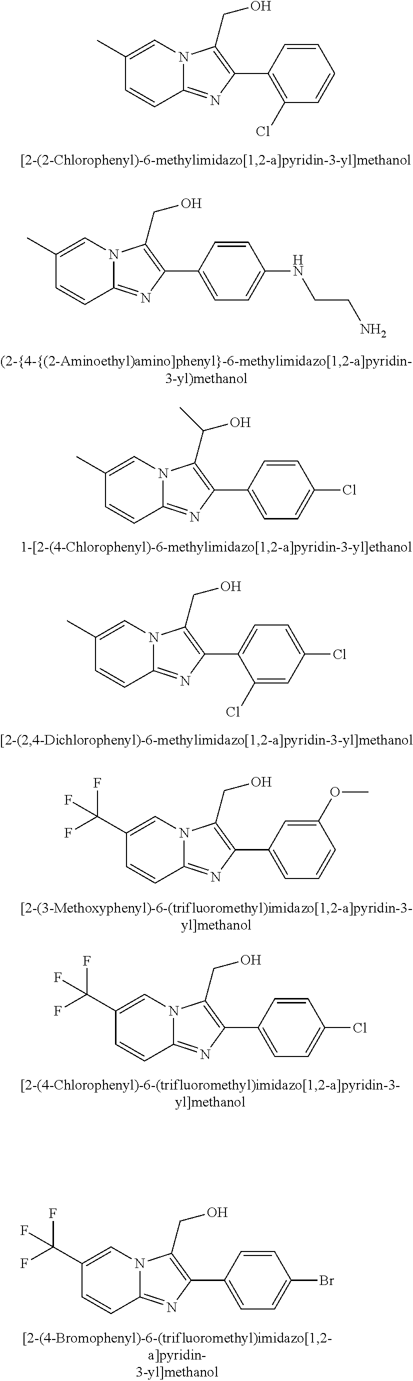

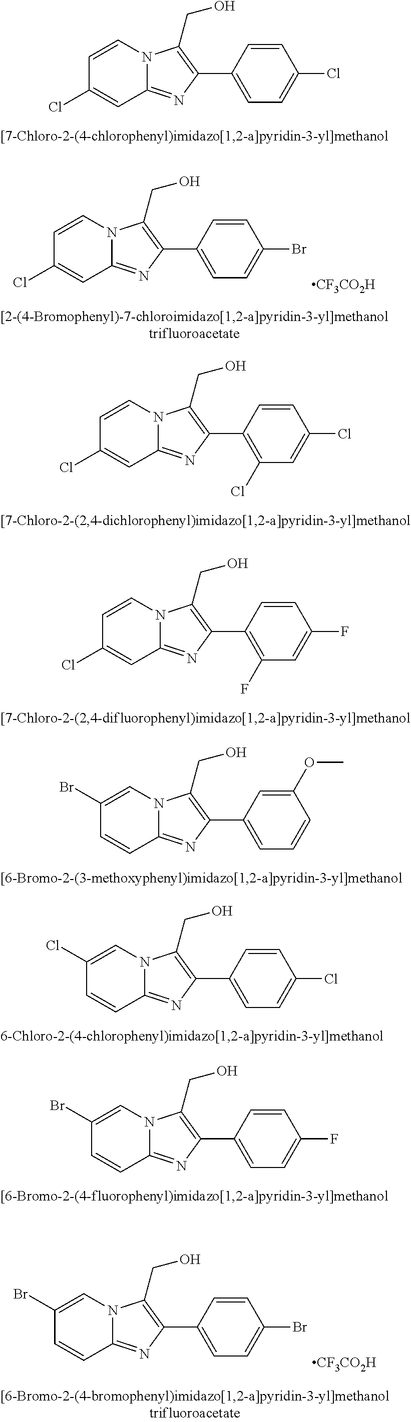

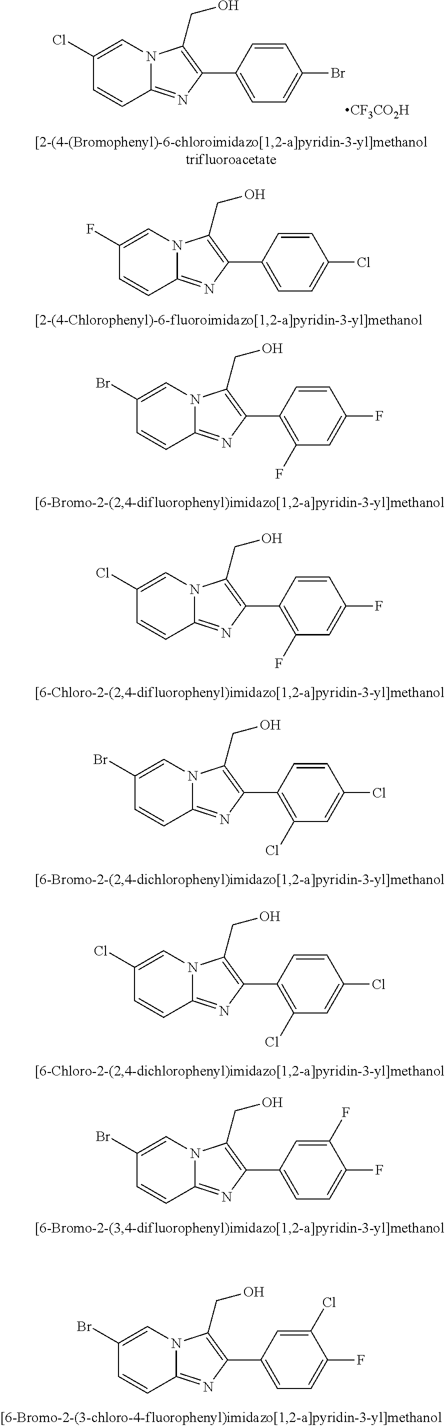

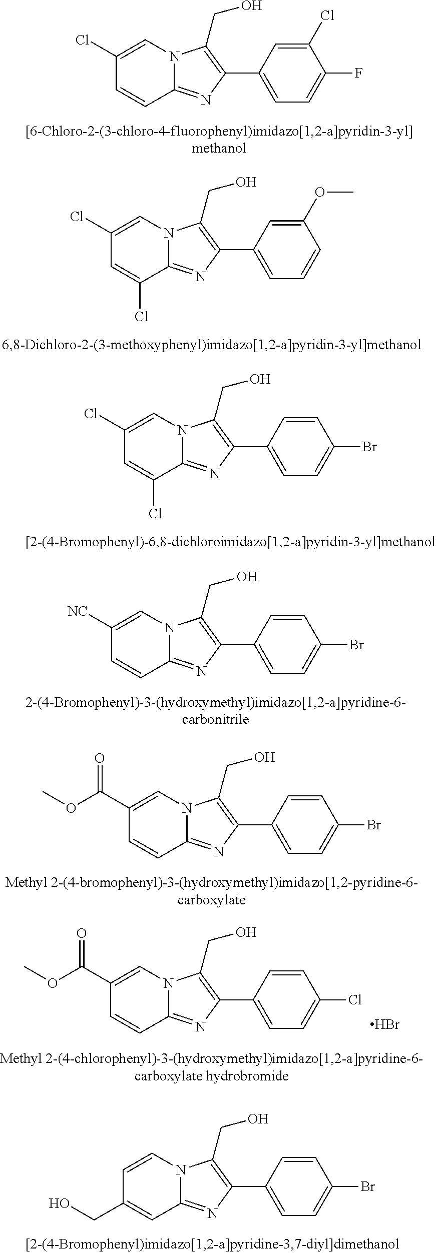

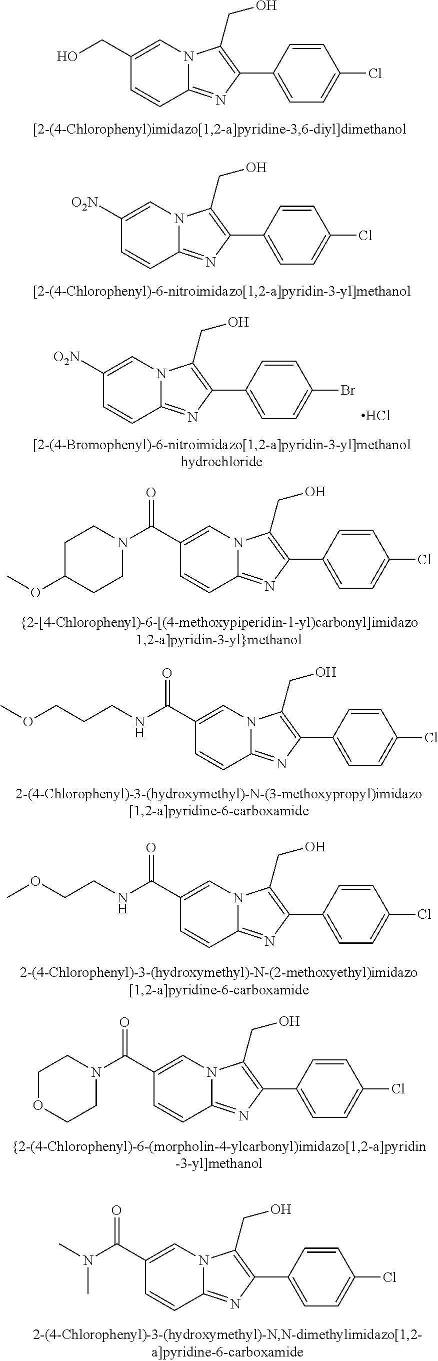

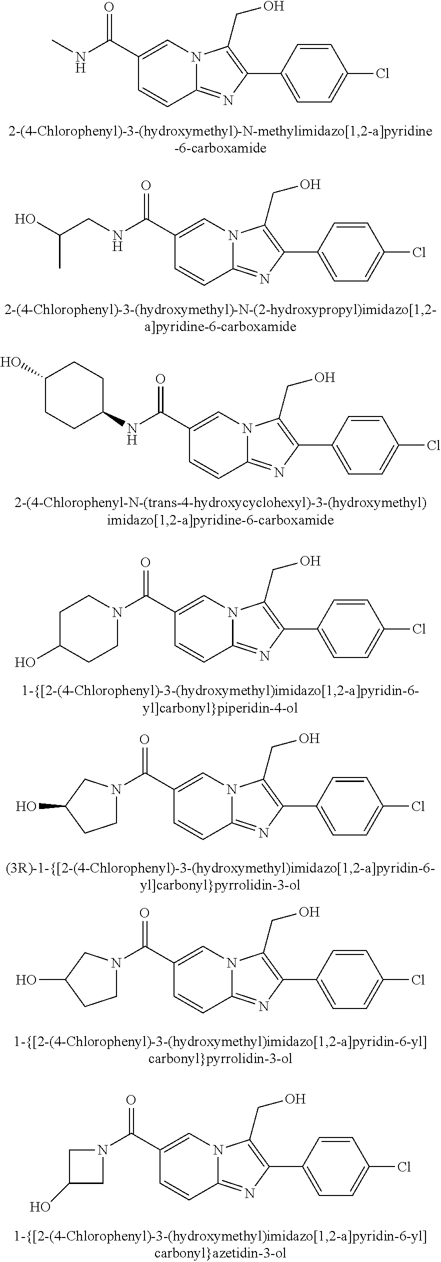

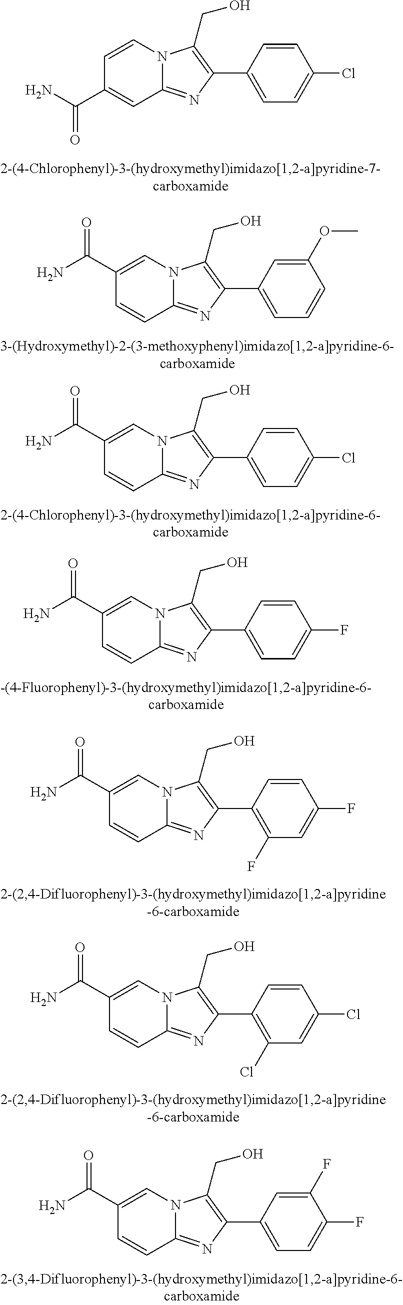

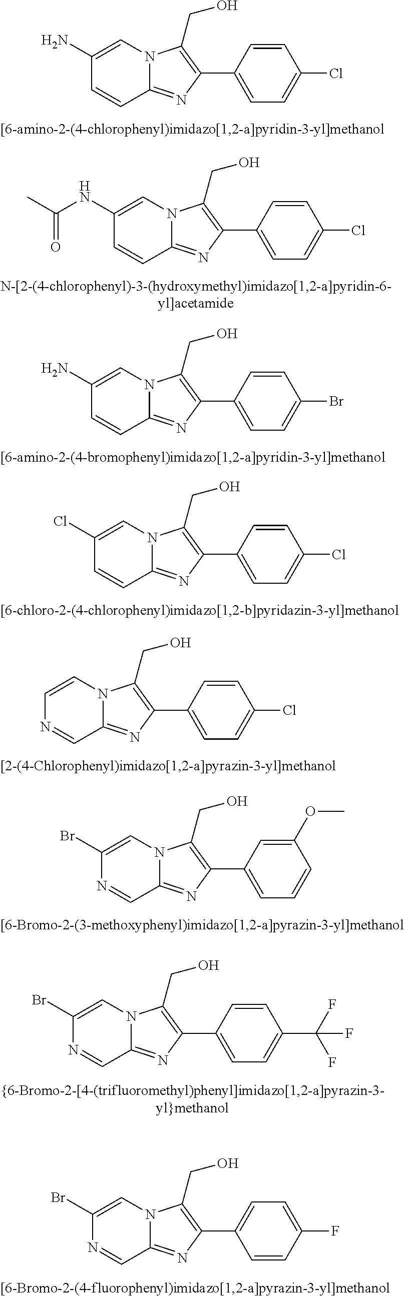

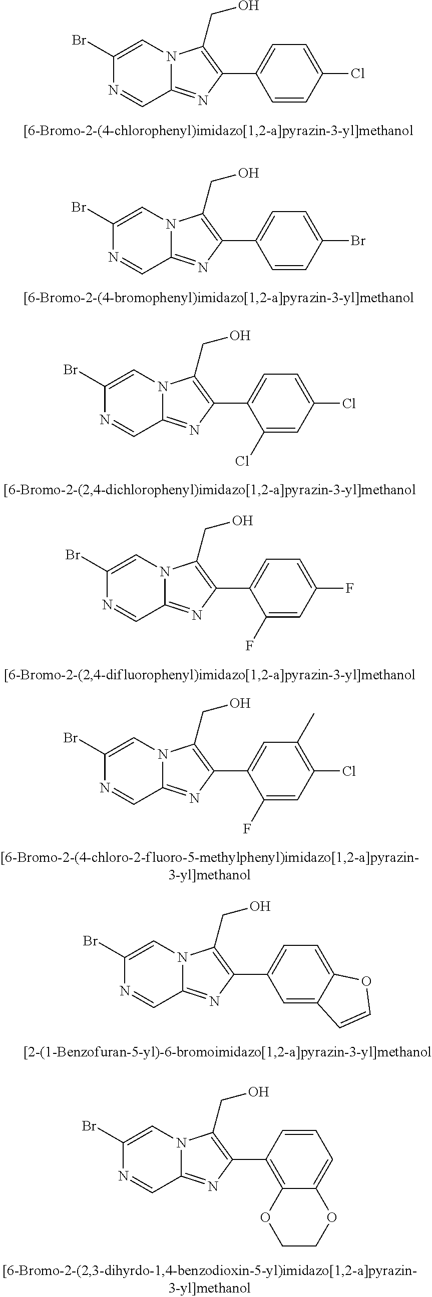

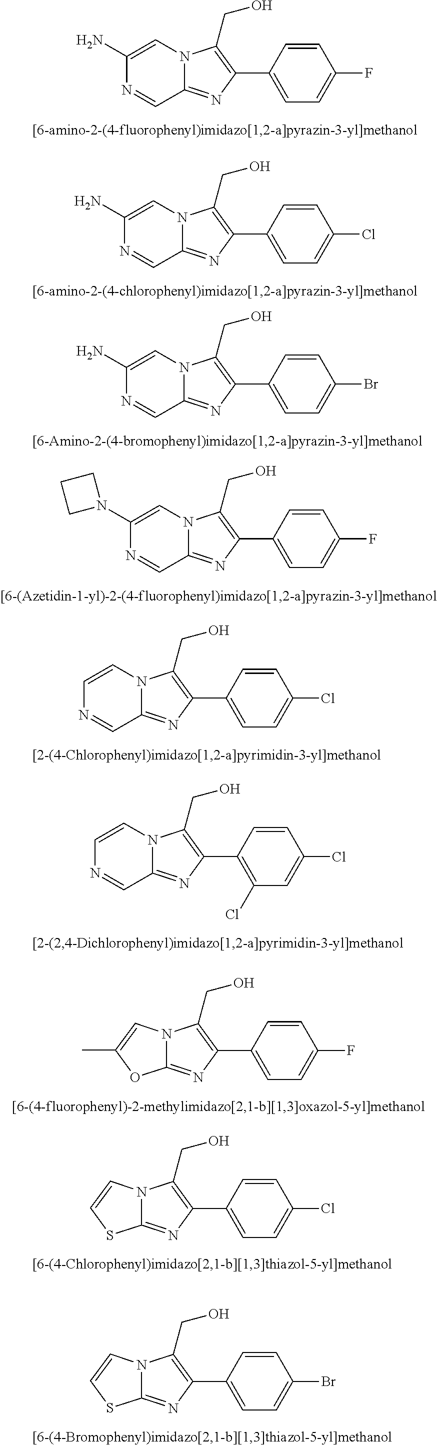

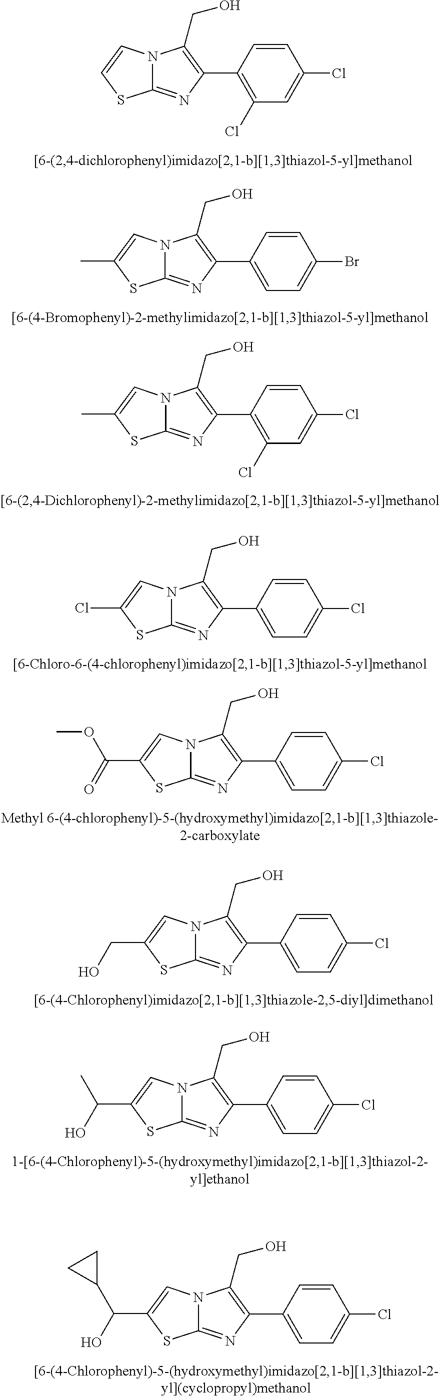

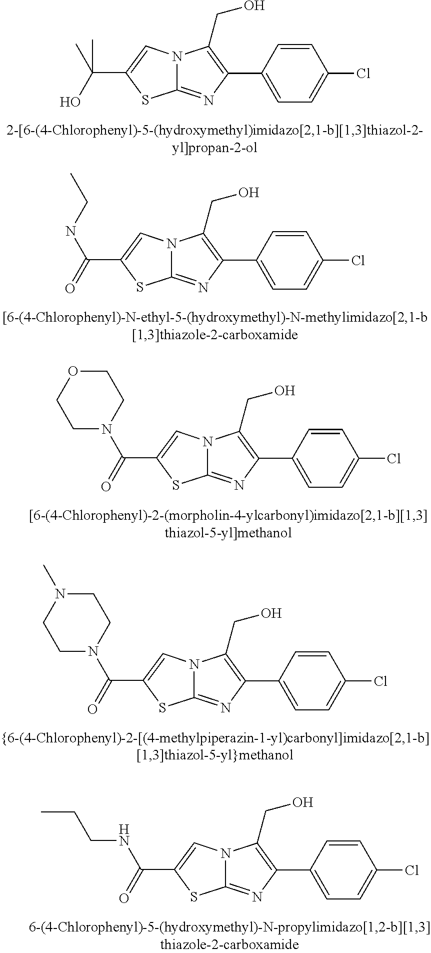 [SMILES: CC(C)(O)c1cn2c(CO)c(-c3ccc(Cl)cc3)nc2s1 ; CCNC(=O)c1cn2c(CO)c(-c3ccc(Cl)cc3)nc2s1 ; O=C(c1cn2c(CO)c(-c3ccc(Cl)cc3)nc2s1)N1CCOCC1 ; CN1CCN(C(=O)c2cn3c(CO)c(-c4ccc(Cl)cc4)nc3s2)CC1 ; CCCNC(=O)c1cn2c(CO)c(-c3ccc(Cl)cc3)nc2s1]

Further specific Examples of VAP-1 compounds include:

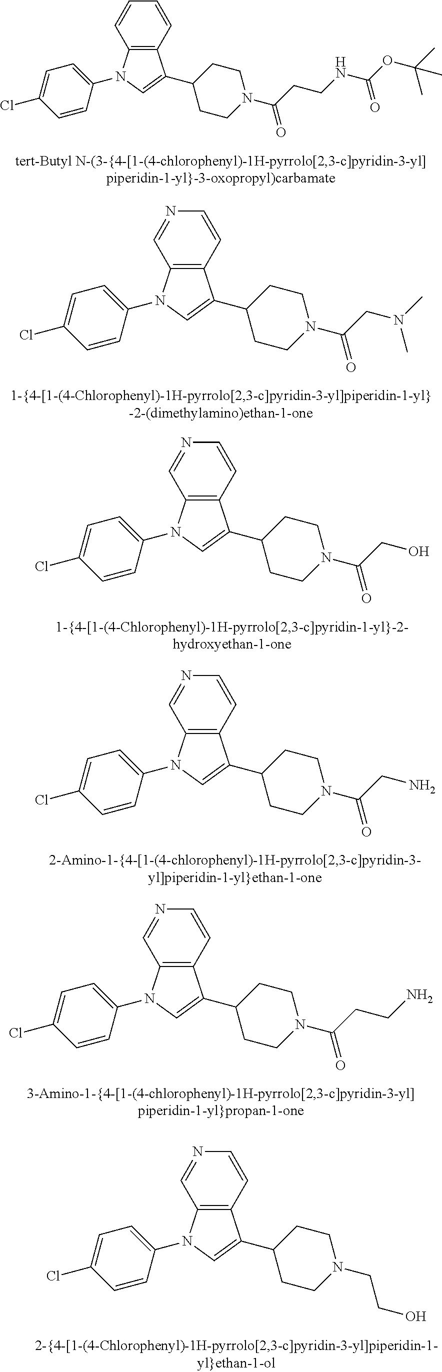 [SMILES: CC(C)(C)OC(=O)NCCC(=O)N1CCC(c2cn(-c3ccc(Cl)cc3)c3ccccc23)CC1 ; CN(C)CC(=O)N1CCC(c2cn(-c3ccc(Cl)cc3)c3cnccc23)CC1 ; O=C(CO)N1CCC(c2cn(-c3ccc(Cl)cc3)c3cnccc23)CC1 ; NCC(=O)N1CCC(c2cn(-c3ccc(Cl)cc3)c3cnccc23)CC1 ; NCCC(=O)N1CCC(c2cn(-c3ccc(Cl)cc3)c3cnccc23)CC1 ; OCCN1CCC(c2cn(-c3ccc(Cl)cc3)c3cnccc23)CC1]

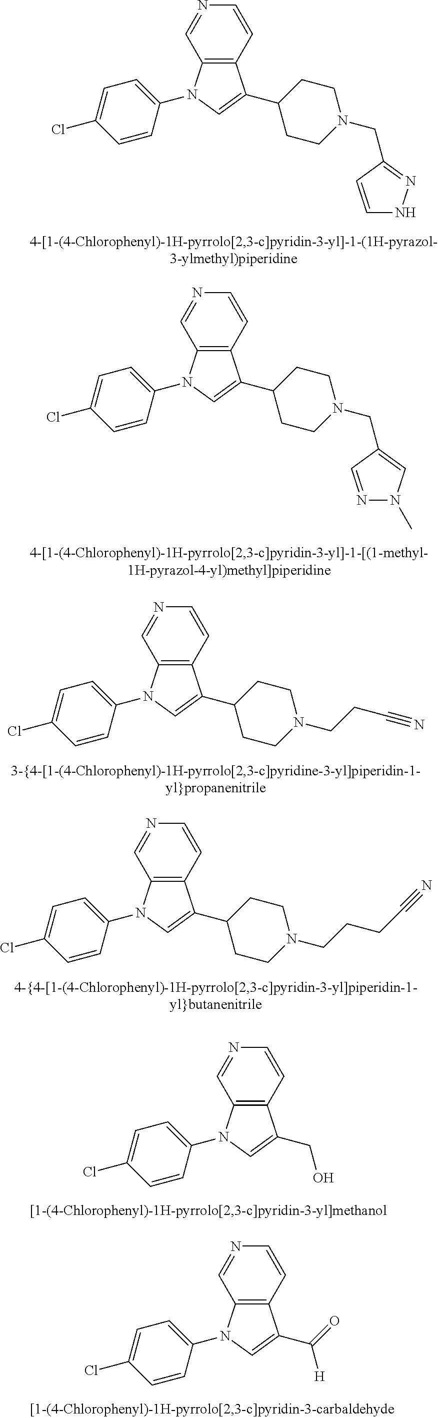 [SMILES: Clc1ccc(-n2cc(C3CCN(Cc4cc[nH]n4)CC3)c3ccncc32)cc1 ; Cn1cc(CN2CCC(c3cn(-c4ccc(Cl)cc4)c4cnccc34)CC2)cn1 ; N#CCCN1CCC(c2cn(-c3ccc(Cl)cc3)c3cnccc23)CC1 ; N#CCCCN1CCC(c2cn(-c3ccc(Cl)cc3)c3cnccc23)CC1 ; OCc1cn(-c2ccc(Cl)cc2)c2cnccc12 ; O=Cc1cn(-c2ccc(Cl)cc2)c2cnccc12]

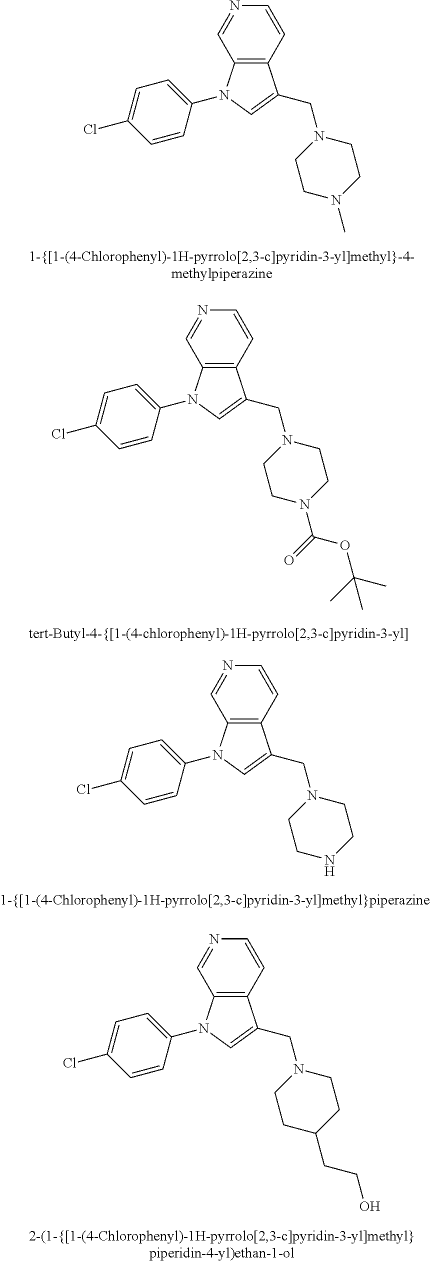 [SMILES: CN1CCN(Cc2cn(-c3ccc(Cl)cc3)c3cnccc23)CC1 ; CC(C)(C)OC(=O)N1CCN(Cc2cn(-c3ccc(Cl)cc3)c3cnccc23)CC1 ; Clc1ccc(-n2cc(CN3CCNCC3)c3ccncc32)cc1 ; OCCC1CCN(Cc2cn(-c3ccc(Cl)cc3)c3cnccc23)CC1]

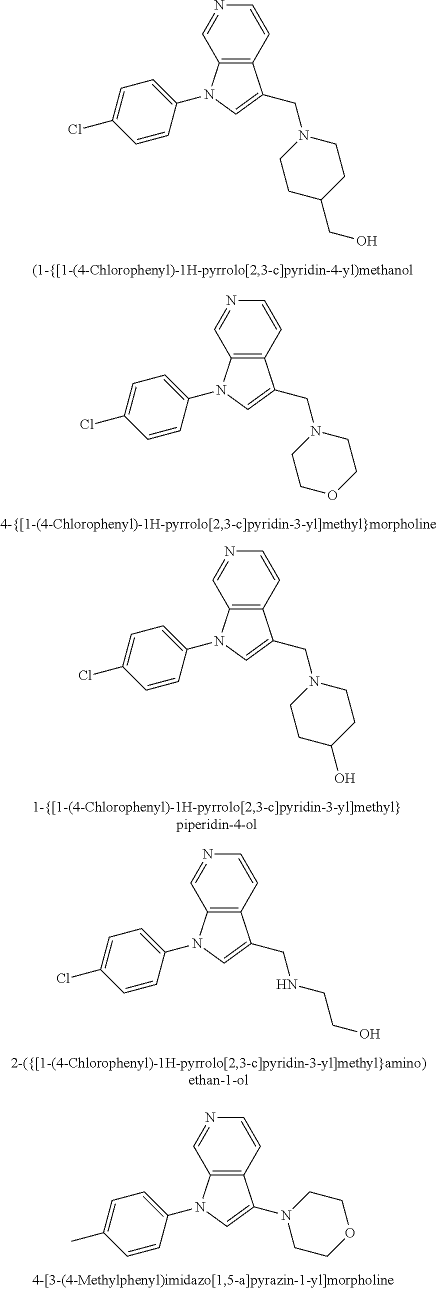 [SMILES: OCC1CCN(Cc2cn(-c3ccc(Cl)cc3)c3cnccc23)CC1 ; Clc1ccc(-n2cc(CN3CCOCC3)c3ccncc32)cc1 ; OC1CCN(Cc2cn(-c3ccc(Cl)cc3)c3cnccc23)CC1 ; OCCNCc1cn(-c2ccc(Cl)cc2)c2cnccc12 ; Cc1ccc(-n2cc(N3CCOCC3)c3ccncc32)cc1]

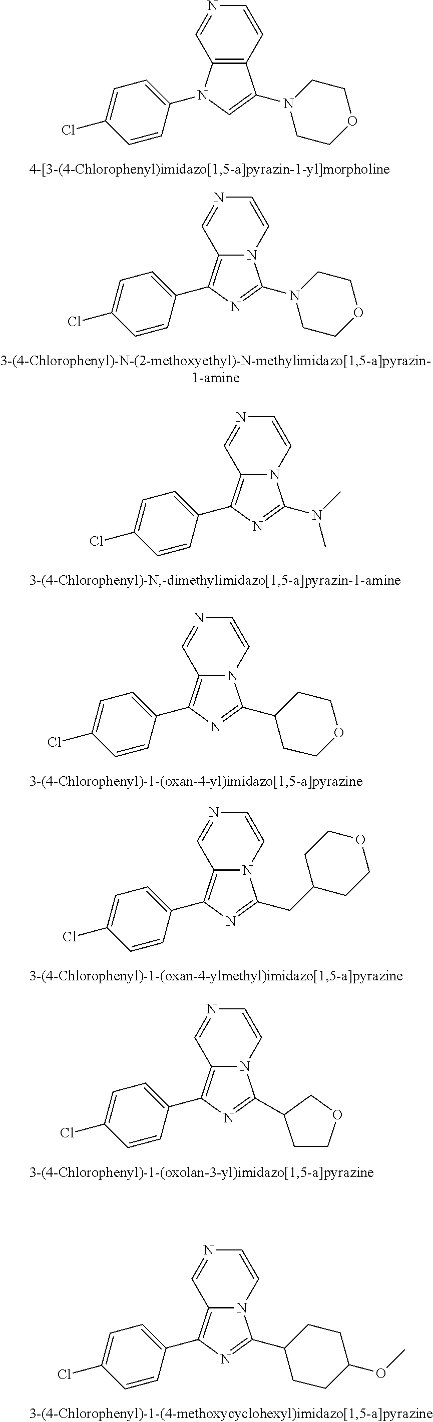

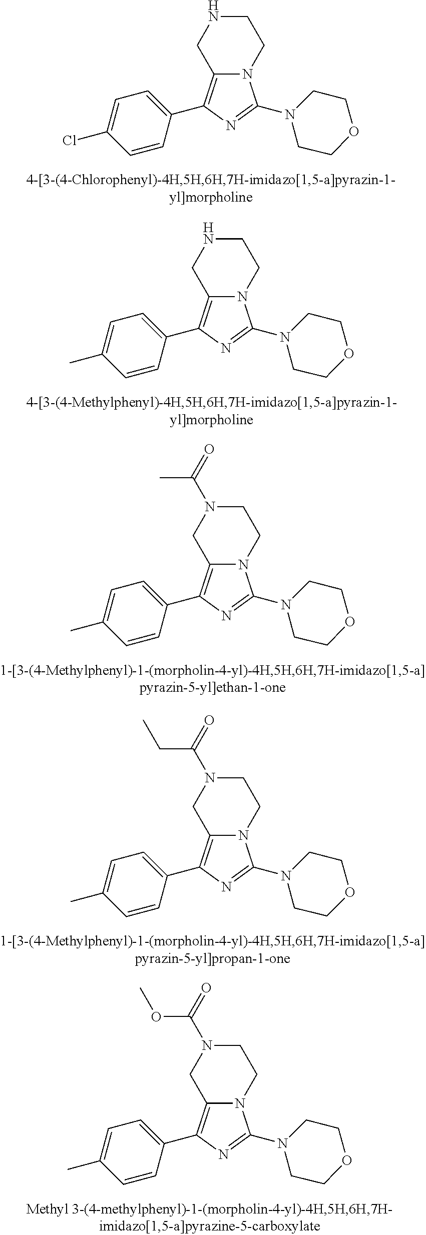 [SMILES: Clc1ccc(-c2nc(N3CCOCC3)n3c2CNCC3)cc1 ; Cc1ccc(-c2nc(N3CCOCC3)n3c2CNCC3)cc1 ; CC(=O)N1CCn2c(N3CCOCC3)nc(-c3ccc(C)cc3)c2C1 ; CCC(=O)N1CCn2c(N3CCOCC3)nc(-c3ccc(C)cc3)c2C1 ; COC(=O)N1CCn2c(N3CCOCC3)nc(-c3ccc(C)cc3)c2C1]

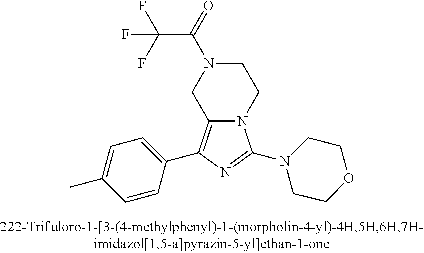 [SMILES: Cc1ccc(-c2nc(N3CCOCC3)n3c2CN(C(=O)C(F)(F)F)CC3)cc1]

In an embodiment, the VAP-1 inhibitor suitable for use in the present invention is selected from the group consisting of:

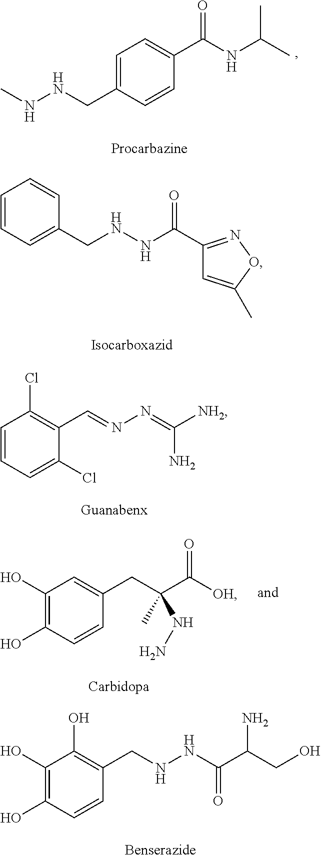 [SMILES: CNNCc1ccc(C(=O)NC(C)C)cc1 ; Cc1cc(C(=O)NNCc2ccccc2)no1 ; NC(N)=N/N=C/c1c(Cl)cccc1Cl ; C[C@@](Cc1ccc(O)c(O)c1)(NN)C(=O)O ; NC(CO)C(=O)NNCc1ccc(O)c(O)c1O]

and pharmaceutically acceptable salts thereof.

Racemic Carbidopa is useful in the present invention. Preferably the Carbidopa for use in the invention is the (R) enantiomer or the (S) enantiomer.

Racemic Benserazide is preferred for use in the present invention. In an embodiment the Benserazide for use in the present invention is the (R) enantiomer or the (S) enantiomer.

In a particular embodiment of the invention, there is provided benserazide, or a pharmaceutically acceptable salt thereof, for use in the treatment of muscular dystrophy, particularly Duchenne muscular dystrophy, in a human subject.

### Compositions

For clinical use, the VAP-1 inhibitor compounds of the invention are formulated into pharmaceutical formulations for various modes of administration. It will be appreciated that compounds may be administered together with a physiologically acceptable carrier, excipient, or diluent. The pharmaceutical compositions of the invention may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Formulations may conveniently be presented in unit dosage form, e.g., tablets and sustained release capsules, and in liposomes, and may be prepared by any method known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Such a dosage may be given orally or parenterally.

### VAP-1 Inhibition Assay

This assay is performed at room temperature with purified recombinantly expressed human VAP-1 (SSAO). Enzyme was prepared essentially as described in Ohman et al. (Protein Expression and Purification 46 (2006) 321-331). The enzyme activity is assayed with benzylamine as substrate by measuring either benzaldehyde production, using 14C-labeled substrate, or by utilizing the production of hydrogen peroxide in a horseradish peroxidise (HRP) coupled reaction. Briefly, test compounds are dissolved in dimethyl sulfoxide (DMSO) to a concentration of 10 mM. Dose-response measurements are assayed by either creating 1:10 serial dilutions in DMSO to produce a 7 point curve or by making 1:3 serial dilutions in DMSO to produce 11 point curves. The top concentrations are adjusted depending on the potency of the compounds and subsequent dilution in reaction buffer yielded a final DMSO concentration ≦2%.

Hydrogen peroxide detection: In a horseradish peroxidise (HRP) coupled reaction, hydrogen peroxide oxidation of 10-acetyl-3,7-dihydroxyphenoxazine produces resorufin, which is a highly fluorescent compound (Zhout and Panchuk-Voloshina. Analytical Biochemistry 253 (1997) 169-174; AmplexR Red Hydrogen Peroxide/peroxidise Assay kit, Invitrogen A22188). Enzyme and compounds in 50 mM sodium phosphate, pH 7.4 are set to pre-incubate in flat-bottomed microtiter plates for approximately 15 minutes before initiating the reaction by addition of a mixture of HRP, benzylamine and Amplex reagent. Benzylamine concentration is fixed at a concentration corresponding to the Michaelis constant, determined using standard procedures. Fluorescence intensity is then measured at several time points during 1-2 hours, exciting at 544 nm and reading the emission at 590 nm. For the human SSAO assay final concentrations of the reagents in the assay wells are: SSAO enzyme 1 mg/ml, benzylamine 100 μM, Amplex reagent 20 μM, HRP 0.1 U/mL and varying concentrations of test compound. The inhibition is measured as % decrease of the signal compared to a control without inhibitor (only diluted DMSO). The background signal from a sample containing no SSAO enzyme is subtracted from all data points. Data is fitted to a four parameter logistic model and IC50 values are calculated, for example by using the GraphPad Prism 4 or XLfit 4 programs.

### Aldehyde Detection:

SSAO activity is assayed using 14C-labeled benzylamine and analysed by measuring radioactive benzaldehyde. In a white 96-well optiplate (Packard), 20 μL of diluted test compound is pre-incubated at rt with 20 μL SSAO enzyme for approximately 15 minutes with continuous agitation. All dilutions are made with PBS. The reaction is initiated by adding 20 μL of the benzylamine substrate solution containing [7-14C] Benzylamine hydrochloride (CFA589, GE Healthcare). The plate is incubated for 1 hour as above after which the reaction is stopped by acidification (10 μL 1 M HCl). Then 90 μL Micro Scint-E solution (Perkin-Elmer) is added to each well and the plate is continuously mixed for 15 minutes. Phase separation occurs and activity is read in a scintillation counter (eg Topcount, Perkin-Elmer). In the final reaction well, human recombinant SSAO concentration is 10 μg/ml. In order to optimize sensitivity, the substrate concentration is decreased as compared to the HRP coupled assay in order to get a higher fraction of radioactive product. In the human SSAO assay, benzylamine concentration is 40 μM (0.2 Ci/mL). Data is analysed as above.

Embodiments of the invention are described below, with reference to the accompanying drawings in which:

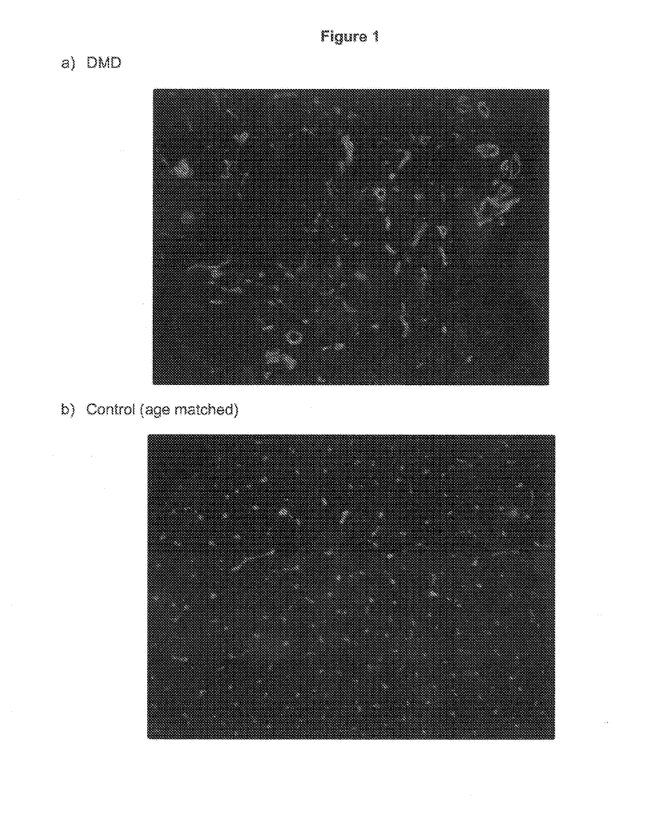

FIG. 1 shows: (a) VAP-1 expression in a muscle tissue section of a boy with Duchenne Muscular Dystrophy (DMD); and (b) VAP-1 expression in a muscle tissue section of an age-matched boy with normal muscles;

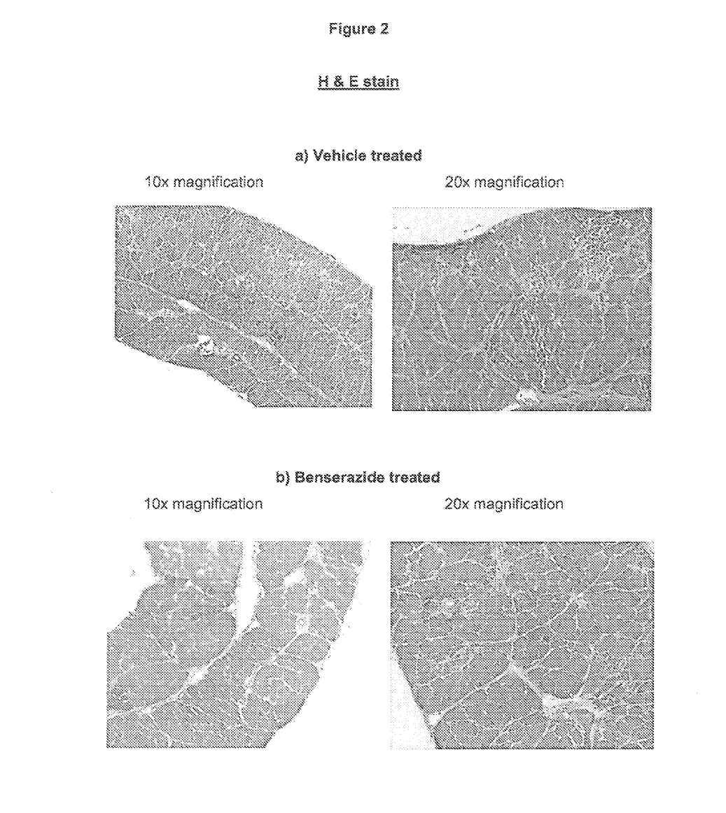

FIG. 2 shows, at ten times and twenty times magnification, hematoxylin and eosin (H & E) staining of sections of diaphragms of mdx mice treated with: (a) vehicle; or (b) benserazide; and

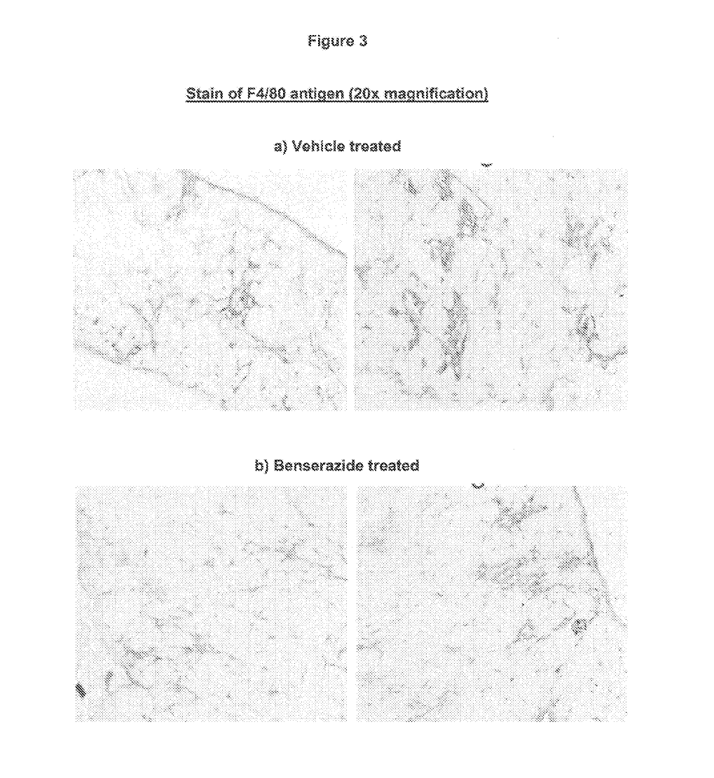

FIG. 3 shows, at twenty times magnification, staining of murine F4/80 antigen in sections of diaphragms of mdx mice treated with: (a) vehicle; or (b) benserazide.

### EXAMPLE 1

Studies in to the overexpression of VAP-1 in dystrophic muscle tissue are on-going in tissue sections derived from patients with muscular dystrophy.

In these on-going studies, the increased expression of VAP-1 in the tissue section (detected with a goat anti-human VAP-1 antibody (Everest) followed by Cy3 labelled anti-goat IgG and imaged using a confocal microscope) and a monoclonal rat anti mouse antibody followed by a Cy3 labelled anti-rat antibody is revealed when compared to non-dystrophic control tissue.

In further on-going experiments the effect of VAP-1/SSAO inhibitors including carbidopa is being examined in the mdx and dy/dy mouse models of muscular dystrophy. In these models groups of mice were dosed once per day with carbidopa (25 mg/kg p.o.) for up to 12 weeks. The degree of inflammation and fibrosis in the muscle was then examined.

### EXAMPLE 2

### VAP-1 Expression is Increased in the Muscle of a Patient with Duchenne Muscular Dystrophy (DMD)

The expression of VAP-1 in a muscle tissue section of a boy with Duchenne Muscular Dystrophy (DMD) was compared with VAP-1 expression in a muscle tissue section of an age-matched boy with normal muscles as a control. VAP-1 expression was detected with a monoclonal rat anti-mouse VAP-1 antibody, followed by a Cy3-labelled anti-rat IgG antibody, and imaged using a confocal microscope. The results are shown in FIG. 1.

FIG. 1(a) shows VAP-1 expression in the DMD tissue section, and FIG. 1(b) shows VAP-1 expression in the age-matched control. VAP-1 expression is greatly increased in the DMD tissue section.

### EXAMPLE 3

### Effect of the VAP-1 Inhibitor Benserazide on Diaphragm Muscle in a Mouse Model of Muscular Dystrophy

Duchenne muscular dystrophy (DMD) is an X-linked muscle disease. Patients develop progressive weakness of skeletal and respiratory muscles and dilated cardiomyopathy. Clinical onset is usually between 2 and 5 years of age. Most patients loose independent ambulation in their teens, after which scoliosis develops. Death usually occurs before forty years of age and is most often the result of respiratory or cardiac failure. The biochemical cause of DMD is a severe deficiency of dystrophin, an essential component of the sarcolemmal dystrophin-associated glycoprotein complex. When complex assembly is disturbed, the linkage between the muscle cell's cytoskeleton and the extracellular matrix is compromised, leading to sarcolemmal instability and increased vulnerability to mechanical stress. Fibres undergo necrosis by excessive Ca²⁺ influx and are progressively replaced by connective and adipose tissue.

The immune system plays a pivotal role in the pathogenesis of DMD. Contraction of dystrophin deficient myofibres produces severe damage and generates cycles of muscle fibre necrosis and regeneration. Necrotizing myofibres are attacked by macrophages; inflammatory cells are present throughout the endomysial, perimysial, and perivascular areas. Macrophages are the most abundant immune cells observed in DMD muscle and both proinflammatory M1 phenotype macrophages and regeneration-focussed M2 phenotype macrophages are present. Within the inflammatory areas, few T cells, B cells, and dendritic cells are also present. Infiltrating T cells are predominantly CD4+, and smaller numbers of CD8+Tcells can be found. The T cell receptor repertoire of CD4+ and CD8+ T cells does not display dominant Va or VP rearrangements, which points toward a nonspecific cell recruitment to sites of muscle fibre destruction. In addition to their involvement in muscle damage, T cells also play an important role in the fibrotic processes present in dystrophic muscle. T cell deficiency significantly reduces collagen matrix accumulation in the murine model. The build up of the inflammatory response is regulated through interactions between adhesion molecules, receptors, and soluble factors, recruiting immune cells from the blood stream to the muscle tissue.

The most studied animal model for DMD is the mdx mouse. This was first described by Bulfield et al (Proc. Natl. Acad. Sci. USA, 1984, 81:1189-1192). It has a point mutation within its dystrophin gene, and as a result the mouse has no functional dystrophin in its muscles. Early in life, the mdx mouse exhibits phases of marked skeletal muscle degeneration and subsequent regeneration. As it ages, certain muscle types (including the diaphragm) show weakness and increased fibrosis. The mdx mouse diaphragm reproduces the degenerative changes of DMD, exhibiting a pattern of degeneration, fibrosis and severe functional deficit comparable to that of DMD limb muscle. This provides a quantitative framework for studying the pathogenesis of dystrophy (Stedman et al, Nature, 1991, 352, 536-539).

12 week old mdx mice were treated with benserazide (20 mg/kg, po, once per day) or vehicle (water, once per day), in groups of 8 mice. After 6 weeks of treatment, diaphragms of the mice were collected and flash frozen in liquid nitrogen-cooled isopentane. The sections were stored on slides at −20° C. until required.

Hematoxylin and eosin (H & E) staining was used to show cytoplasmic, nuclear, and extracellular matrix features. Hematoxylin stains nucleic acids, and eosin stains proteins nonspecifically. Staining of F4/80 antigen (a glycoprotein expressed by murine macrophages) was used to show macrophages. The results of H & E staining are shown in FIG. 2, and the results of staining of murine F4/80 antigen are shown in FIG. 3.

The H & E staining in FIG. 2 shows an approximate 50% reduction in inflammatory infiltrates in mice treated with benserazide compared to vehicle. The F4/80 staining in FIG. 3 also shows an approximate 50% reduction in macrophage infiltration in mice treated with benserazide compared to vehicle.

These results show that the VAP-1 inhibitor benserazide reduces the inflammatory response to muscle damage in dystrophic mice. It is known from the mdx mouse model that partial inhibition of macrophage incursion into the muscle tissue has a beneficial effect on muscle tissue maintenance. Thus, this example shows that the VAP-1 inhibitor benserazide can be used for the treatment of dystrophic muscle, and muscular dystrophy.

1-2. (canceled)

3. A method of treating muscular dystrophy comprising administering to a subject in need of such treatment an effective amount of a VAP-1 inhibitor compound.

4-5. (canceled)

6. The method according to claim 3 wherein the VAP-1 inhibitor compound is a polypeptide or protein.

7. The method according to claim 3 wherein the muscular dystrophy is Duchenne muscular dystrophy, Becker muscular dystrophy, limb girdle muscular dystrophy, congenital muscular dystrophy, or distal muscular dystrophy.

8. (canceled)

9. The method of claim 3, wherein the VAP-1 inhibitor compound is:
2,2,2-Trichloroethyl 4-isopropyl-1,4,6,7-tetrahydro-5H-imidazo[4,5-c]pyridine-5-carboxylate;
2-Chloro-2,2-difluoroethyl 4-isopropyl-1,4,6,7-tetrahydro-5H-imidazo[4,5-c]pyridine-5-carboxylate;
Benzyl 4-isopropyl-1,4,6,7-tetrahydro-5H-imidazo[4,5-c]pyridine-5-carboxylate;
3-Chlorobenzyl 4-isopropyl-1,4,6,7-tetrahydro-5H-imidazo[4,5-c]pyridine-5-carboxylate;
4-Chlorobenzyl 4-isopropyl-1,4,6,7-tetrahydro-5H-imidazo[4,5-c]pyridine-5-carboxylate;
Pyridin-2-ylmethyl 4-isopropyl-1,4,6,7-tetrahydro-5H-imidazo[4,5-c]pyridine-5-carboxylate;
Pyridin-3-ylmethyl 4-isopropyl-1,4,6,7-tetrahydro-5H-imidazo[4,5-c]pyridine-5-carboxylate;
Pyridin-4-ylmethyl 4-isopropyl-1,4,6,7-tetrahydro-5H-imidazo[4,5-c]pyridine-5-carboxylate;
(5-Chloropyridin-2-yl)methyl 4-isopropyl-1,4,6,7-tetrahydro-5H-imidazo[4,5-c]pyridine-5-carboxylate;
Pyrazin-2-ylmethyl 4-isopropyl-1,4,6,7-tetrahydro-5H-imidazo[4,5-c]pyridine-5-carboxylate;
Benzyl (4S,6S)-6-(aminocarbonyl)-4-isopropyl-1,4,6,7-tetrahydro-5H-imidazo-[4,5-c]pyridine-5-carboxylate;
Benzyl (4S,6S)-4-isopropyl-6-[(methylamino)carbonyl]-1,4,6,7-tetrahydro-5H-imidazo[4,5-c]pyridine-5-carboxylate;
5-Benzyl 6-methyl (4S,6S)-4-isopropyl-1,4,6,7-tetrahydro-5H-imidazo[4,5-c]pyridine-5,6-dicarboxylate;
2-Phenoxyethyl 4-isopropyl-1,4,6,7-tetrahydro-5H-imidazo[4,5-c]pyridine-5-carboxylate;
2-(4-Chlorophenoxy)ethyl 4-isopropyl-1,4,6,7-tetrahydro-5H-imidazo[4,5-c]pyridine-5-carboxylate;
(3 S)-Tetrahydrofuran-3-yl(4S)-4-isopropyl-1,4,6,7-tetrahydro-5H-imidazo[4,5-c]pyridine-5-carboxylate;
Tetrahydrofuran-3-ylmethyl 4-isopropyl-1,4,6,7-tetrahydro-5H-imidazo[4,5-c]pyridine-5-carboxylate;
(3-Methyloxetan-3-yl)methyl 4-isopropyl-1,4,6,7-tetrahydro-5H-imidazo[4,5-c]pyridine-5-carboxylate;
2-(Dimethylamino)ethyl 4-isopropyl-1,4,6,7-tetrahydro-5H-imidazo[4,5-c]pyridine-5-carboxylate;
(2R)-Tetrahydrofuran-2-ylmethyl 4-isopropyl-1,4,6,7-tetrahydro-5H-imidazo[4,5-c]pyridine-5-carboxylate;
1,3-Thiazol-2-ylmethyl 4-isopropyl-1,4,6,7-tetrahydro-5H-imidazo[4,5-c]pyridine-5-carboxylate;
(5-Methylisoxazol-3-yl)methyl 4-isopropyl-1,4,6,7-tetrahydro-5H-imidazo[4,5-c]pyridine-5-carboxylate;
[(2S)-1-Methylpyrrolidin-2-yl]methyl 4-isopropyl-1,4,6,7-tetrahydro-5H-imidazo-[4,5-c]pyridine-5-carboxylate;
(3R)-1-methylpyrrolidin-3-yl 4-isopropyl-1,4,6,7-tetrahydro-5H-imidazo[4,5-c]pyridine-5-carboxylate;
Oxetan-2-ylmethyl 4-isopropyl-1,4,6,7-tetrahydro-5H-imidazo[4,5-c]pyridine-5-carboxylate;
2-(Pyridin-3-yloxy)ethyl 4-isopropyl-1,4,6,7-tetrahydro-5H-imidazo[4,5-c]pyridine-5-carboxylate;
2-(2,2,2-Trifluoroethoxy)ethyl 4-isopropyl-1,4,6,7-tetrahydro-5H-imidazo[4,5-c]pyridine-5-carboxylate;
or a pharmaceutically acceptable salt thereof.

10. The method of claim 3, wherein the VAP-1 inhibitor compound is:
3-(4-Chlorophenyl)-1-(oxolan-3-ylmethyl)-1H-pyrrolo[3,2-c]pyridine;
tert-Butyl 4-[3-(4-chlorophenyl)-1H-pyrrolo[3,2-c]pyridin-1-yl]piperidine-1-carboxylate;
3-(4-Chlorophenyl)-1-(oxolan-3-yl)-1H-pyrrolo[3,2-c]pyridine;
3-(4-Chlorophenyl)-1-(oxan-4-yl)-1H-pyrrolo[3,2-c]pyridine;
3-(4-chlorophenyl)-1H-pyrrolo[3,2-c]pyridine;
4-[3-(3,4-Dichlorophenyl)-1H-pyrrolo[3,2-c]pyridin-1-yl]piperidine;
1-{4-[3-(3,4-Dichlorophenyl)-1H-pyrrolo[3,2-c]pyridin-1-yl]piperidin-1-yl}-2-hydroxyethan-1-one;
4-[1-(4-Chlorophenyl)-1H-pyrrolo[2,3-c]pyridin-3-yl];
tert-Butyl 4-[1-(4-chlorophenyl)-1H-pyrrolo[2,3-c]pyridin-3-yl]piperidine-1-carboxylate;
1-(4-Chlorophenyl)-3-piperidin-4-yl-1H-pyrrolo[2,3-c]pyridine;
tert-Butyl N-{4-[1-(4-chlorophenyl)-1H-pyrrolo[2,3-c]pyridin-3-yl]cyclohexyl}-carbamate;
4-[1-(4-Chlorophenyl)-1H-pyrrolo[2,3-c]pyridin-3-yl]cyclohexan-1-amine;
4-[1-(4-Chloro-2-methylphenyl)-1H-pyrrolo[2,3-c]pyridin-3-yl]cyclohexan-1-amine;
1-{4-[1-(4-Chlorophenyl)-1H-pyrrolo[2,3-c]pyridin-3-yl]piperidin-1-yl}-2-(dimethyl-amino)ethan-1-one;
1-{4-[1-(4-Chlorophenyl)-1H-pyrrolo[2,3-c]pyridine-3-yl]piperidin-1-yl}-2-hydroxyethan-1-one;
2-Amino-1-{4-[1-(4-chlorophenyl)-1H-pyrrolo[2,3-c]pyridin-3-yl]piperidin-1-yl}ethan-1-one hydrochloride;
3-Amino-1-{4-[1-(4-chlorophenyl)-1H-pyrrolo[2,3-c]pyridin-3-yl]piperidin-1-yl}propan-1-one hydrochloride;
2-{4-[1-(4-Chlorophenyl)-1H-pyrrolo[2,3-c]pyridin-3-yl]piperidin-1-yl}ethan-1-ol;
4-[1-(4-Chlorophenyl)-1H-pyrrolo[2,3-c]pyridin-3-yl]-1-(1H-pyrazol-3-ylmethyl)-piperidine;
4-[1-(4-Chlorophenyl)-1H-pyrrolo[2,3-c]pyridin-3-yl]-1-[(1-methyl-1H-pyrazol-4-yl)-methyl]piperidine;
3-{4-[1-(4-Chlorophenyl)-1H-pyrrolo[2,3-c]pyridine-3-yl]piperidin-1-yl}propanenitrile hydrochloride;
4-{4-[1-(4-Chlorophenyl)-1H-pyrrolo[2,3-c]pyridin-3-yl]piperidin-1-yl}butanenitrile hydrochloride;
[1-(4-Chlorophenyl)-1H-pyrrolo[2,3-c]pyridin-3-yl]methanol;
1-{[1-(4-Chlorophenyl)-1H-pyrrolo[2,3-c]pyridin-3-yl]methyl}-4-methylpiperazine;
tert-Butyl 4-{[1-(4-chlorophenyl)-1H-pyrrolo[2,3-c]pyridin-3-yl];
1-{[1-(4-Chlorophenyl)-1H-pyrrolo[2,3-c]pyridin-3-yl]methyl}piperazine;
2-(1-{[1-(4-Chlorophenyl)-1H-pyrrolo[2,3-c]pyridin-3-yl]methyl}piperidin-4-yl)ethan-1-ol;
(1-{[1-(4-Chlorophenyl)-1H-pyrrolo[2,3-c]pyridin-3-yl]methyl}piperidin-4-yl)methanol;
4-{[1-(4-Chlorophenyl)-1H-pyrrolo[2,3-c]pyridin-3-yl]methyl}morpholine;
1-{[1-(4-Chlorophenyl)-1H-pyrrolo[2,3-c]pyridin-3-yl]methyl}piperidin-4-ol;
2-({[1-(4-Chlorophenyl)-1H-pyrrolo[2,3-c]pyridin-3-yl]methyl}amino)ethan-1-ol;
4-[3-(4-Methylphenyl)imidazo[1,5-a]pyrazin-1-yl]morpholine;
4-[3-(4-Chlorophenyl)imidazo[1,5-a]pyrazin-1-yl]morpholine;
3-(4-Chlorophenyl)-N-(2-methoxyethyl)-N-methylimidazo[1,5-a]pyrazin-1-amine;
3-(4-Chlorophenyl)-N,N-dimethylimidazo[1,5-a]pyrazin-1-amine;
3-(4-Chlorophenyl)-1-(oxan-4-yl)imidazo[1,5-a]pyrazine;
3-(4-Chlorophenyl)-1-(oxan-4-ylmethyl)imidazo[1,5-a]pyrazine;
3-(4-Chlorophenyl)-1-(oxolan-3-yl)imidazo[1,5-a]pyrazine;
3-(4-Chlorophenyl)-1-(4-methoxycyclohexyl)imidazo[1,5-a]pyrazine;
3-(Oxan-4-yl)-1-phenyl-1H-pyrazolo[3,4-c]pyridine;
4-[3-(Oxan-4-yl)-1H-pyrazolo[3,4-c]pyridin-1-yl]benzonitrile;
1-[4-(Difluoromethyl)phenyl]-3-(oxan-4-yl)-1H-pyrazolo[3,4-c]pyridine;
1-(2-Fluoro-4-methylphenyl)-3-(oxan-4-yl)-1H-pyrazolo[3,4-c]pyridine;
1-(4-Chloro-2-fluorophenyl)-3-(oxan-4-yl)-1H-pyrazolo[3,4-c]pyridine;
1-(2,4-Dimethylphenyl)-3-(oxan-4-yl)-1H-pyrazolo[3,4-c]pyridine;
5-Methyl-2-[3-(oxan-4-yl)-1H-pyrazolo[3,4-c]pyridin-1-yl]pyridine;
2-Methyl-5-[3-(oxan-4-yl)-1H-pyrazolo[3,4-c]pyridin-1-yl]pyridine;
1-[1-(4-Chlorophenyl)-1H-pyrazolo[3,4-c]pyridin-3-yl]-3,3-difluoropyrrolidine;
1-[1-(4-Chlorophenyl)-1H-pyrazolo[3,4-c]pyridin-3-yl]pyrrolidin-3-ol;
3-Methoxy-1-[1-(4-methylphenyl)-1H-pyrazolo[3,4-c]pyridin-3-yl]pyrrolidine;
1-[1-(4-Chlorophenyl)-1H-pyrazolo[3,4-c]pyridin-3-yl]piperidine;
1-[1-(4-Chlorophenyl)-1H-pyrazolo[3,4-c]pyridin-3-yl]-4,4-difluoropiperidine;
1-[1-(4-Chlorophenyl)-1H-pyrazolo[3,4-c]pyridin-3-yl]piperidin-4-ol;
1-[1-(4-Methylphenyl)-1H-pyrazolo[3,4-c]pyridin-3-yl]piperidine-4-carboxamide;
4-[1-(4-Fluorophenyl)-1H-pyrazolo[3,4-c]pyridin-3-yl]morpholine;
4-[1-(4-Chlorophenyl)-1H-pyrazolo[3,4-c]pyridin-3-yl]morpholine;
2,2,2-Trifluoroacetic acid; 4-[1-(4-methylphenyl)-1H-pyrazolo[3,4-c]pyridin-3-yl]morpholine;
4-[1-(2-Fluoro-4-methylphenyl)-1H-pyrazolo[3,4-c]pyridin-3-yl]morpholine;
4-[1-(4-Chlorophenyl)-1H-pyrazolo[3,4-c]pyridin-3-yl]-2-methylmorpholine;
4-[1-(4-Chlorophenyl)-1H-pyrazolo[3,4-c]pyridin-3-yl]-3-methylmorpholine;
4-[1-(4-Methylphenyl)-1H-pyrazolo[3,4-c]pyridin-3-yl]-2-(2-methylpropyl)morpholine;
(2S,6R)-4-[1-(4-Chlorophenyl)-1H-pyrazolo[3,4-c]pyridin-3-yl]-2,6-dimethylmorpholine;
3-[1-(4-Methylphenyl)-1H-pyrazolo[3,4-c]pyridin-3-yl]-8-oxa-3-azabicyclo[3.2.1]octane;
2,2-Dimethyl-4-[1-(4-methylphenyl)-1H-pyrazolo[3,4-c]pyridin-3-yl]morpholine;
3,3-Dimethyl-4-[1-(4-methylphenyl)-1H-pyrazolo[3,4-c]pyridin-3-yl]morpholine;
Methyl 4-[1-(4-methylphenyl)-1H-pyrazolo[3,4-c]pyridin-3-yl]morpholine-3-carboxylate;
4-[1-(4-Methylphenyl)-1H-pyrazolo[3,4-c]pyridin-3-yl]-1,4-oxazepane;
4-[1-(4-Methylphenyl)-1H-pyrazolo[3,4-c]pyridin-3-yl]piperazin-2-one;
N-(2-Methoxyethyl)-N-methyl-1-(4-methylphenyl)-1H-pyrazolo[3,4-c]pyridin-3-amine;
1-[1-(4-Chlorophenyl)-1H-pyrazolo[3,4-c]pyridin-3-yl]piperazine;
1-[1-(4-Chlorophenyl)-1H-pyrazolo[3,4-c]pyridin-3-yl]piperidin-4-amine;
{4-[1-(4-Methylphenyl)-1H-pyrazolo[3,4-c]pyridin-3-yl]morpholin-2-yl}methanamine;
tert-Butyl N-(2-methoxyethyl)-N-[1-(4-methylphenyl)-1H-pyrazolo[3,4-c]pyridin-3-yl]carbamate;
1-[1-(4-Methylphenyl)-1H-pyrazolo[3,4-c]pyridin-3-yl]piperidin-4-ol;
1-[1-(4-Chlorophenyl)-1H-pyrazolo[3,4-c]pyridin-3-yl]-4-(1H-pyrazol-3-ylmethyl)piperazine;
tert-Butyl N-(3-{4-[1-(4-chlorophenyl)-1H-pyrazolo[3,4-c]pyridin-3-yl]piperazin-1-yl}-3-oxopropyl)carbamate;
4-[1-(4-Methylphenyl)-1H-pyrazolo[3,4-c]pyridin-3-yl]morpholine-3-carboxamide;
4-[1-(4-Methylphenyl)-1H-pyrazolo[3,4-c]pyridin-3-yl]-3-[(morpholin-4-yl)carbonyl]morpholine;
N-(2-Aminoethyl)-4-[1-(4-methylphenyl)-1H-pyrazolo[3,4-c]pyridin-3-yl]morpholine-3-carboxamide dihydrochloride;
1-(4-Chlorophenyl)-3-(oxan-4-yl)-1H-pyrazolo[3,4-c]pyridine;
1-(4-Methylphenyl)-3-(oxolan-3-yl)-1H-pyrazolo[3,4-c]pyridine;
1-(4-Methylphenyl)-3-(oxan-4-yl)-1H-pyrazolo[3,4-c]pyridine;
1-(4-Fluorophenyl)-3-(oxan-4-yl)-1H-pyrazolo[3,4-c]pyridine;
4-[1-(4-Chlorophenyl)-1H-pyrazolo[3,4-c]pyridin-3-yl]piperidine;
3-[1-(4-Methylphenyl)-1H-pyrazolo[3,4-c]pyridin-3-yl]morpholine;
2-[1-(4-Methylphenyl)-1H-pyrazolo[3,4-c]pyridin-3-yl]morpholine;
5-[1-(4-Methylphenyl)-1H-pyrazolo[3,4-c]pyridin-3-yl]piperidin-2-one;
1-(4-Chlorophenyl)-4-fluoro-3-(oxan-4-yl)-1H-pyrazolo[3,4-c]pyridine;
4-[1-(4-Chlorophenyl)-1H-pyrazolo[3,4-c]pyridin-3-yl]-1-(1H-pyrazol-3-ylmethyl)piperidine;
1-Butyl-4-[1-(4-chlorophenyl)-1H-pyrazolo[3,4-c]pyridin-3-yl]piperidine;
4-[1-(4-Chlorophenyl)-1H-pyrazolo[3,4-c]pyridin-3-yl]-N,N-dimethylpiperidine-1-carboxamide;
Ethyl 4-[1-(4-chlorophenyl)-1H-pyrazolo[3,4-c]pyridin-3-yl]piperidine-1-carboxylate;
3-Amino-1-{4-[1-(4-chlorophenyl)-1H-pyrazolo[3,4-c]pyridin-3-yl]piperidin-1-yl}propan-1-one;
1-(4-Chlorophenyl)-4-methoxy-3-(oxan-4-yl)-1H-pyrazolo[3,4-c]pyridine;
1-(4-Chlorophenyl)-3-(oxan-4-yl)-1H-pyrazolo[3,4-c]pyridin-4-ol;
1-(4-Chlorophenyl)-5-methoxy-3-(oxan-4-yl)-1H-pyrazolo[3,4-c]pyridine;
1-(4-Chlorophenyl)-3-(oxan-4-yl)-1H,5H,6H-pyrazolo[3,4-c]pyridin-5-one;
5-(4-Chlorophenyl)-7-(oxan-4-yl)-5H-pyrrolo[3,2-d]pyrimidine;
1-(4-Chlorophenyl)-3-(oxan-4-yl)-1H-pyrazolo[4,3-d]pyrimidine;
1-(4-Fluorophenyl)-3-(oxan-4-yl)-1H-pyrrolo[2,3-c]pyridine;
1-(4-Chlorophenyl)-3-(oxan-4-yl)-1H-pyrrolo[2,3-c]pyridine;
1-(4-Methylphenyl)-3-(oxan-4-yl)-1H-pyrrolo[2,3-c]pyridine;
5-Chloro-2-[3-(oxan-4-yl)-1H-pyrrolo[2,3-c]pyridin-1-yl]pyridine;
4-[1-(4-Chlorophenyl)-1H-pyrrolo[2,3-c]pyridin-3-yl]morpholine 2,2,2-trifluroacetic acid;
4-amino-1-{4-[1-(4-chlorophenyl)-1H-pyrrolo[2,3-c]pyridin-3-yl]piperidin-1-yl}butan-1-one;
2-Aminoethyl 4-[1-(4-chlorophenyl)-1H-pyrrolo[2,3-c]pyridin-3-yl]piperidine-1-carboxylate;
3-(3,6-Dihydro-2H-pyran-4-yl)-2-methyl-1-(4-methylphenyl)-1H-pyrrolo[2,3-c]pyridine;
or a pharmaceutically acceptable salt thereof.

11. The method of claim 3, wherein the VAP-1 inhibitor compound is:
2,2,2-Trifluoroacetic acid; 2-{4-[1-(4-chlorophenyl)-1H-pyrrolo[2,3-c]pyridin-3-yl]piperidin-1-yl}ethan-1-amine;
3-Aminopropyl 4-[1-(4-chlorophenyl)-1H-pyrrolo[2,3-c]pyridin-3-yl]piperidine-1-carboxylate;
1-{4-[1-(4-Chlorophenyl)-1H-pyrrolo[2,3-c]pyridin-3-yl]piperidin-1-yl}-4-(dimethylamino)butan-1-one; 2,2,2-trifluoroacetic acid;
5-Amino-1-{4-[1-(4-chlorophenyl)-1H-pyrrolo[2,3-c]pyridin-3-yl]piperidin-1-yl}pentan-1-one;
N-(2-Aminoethyl)-4-[1-(4-chlorophenyl)-1H-pyrrolo[2,3-c]pyridin-3-yl]piperidine-1-carboxamide;
N-(3-Aminopropyl)-4-[1-(4-chlorophenyl)-1H-pyrrolo[2,3-c]pyridin-3-yl]piperidine-1-carboxamide;
4-[1-(4-Chlorophenyl)-1H-pyrrolo[2,3-c]pyridin-3-yl]-N-[3-(dimethylamino)propyl]piperidine-1-carboxamide;
1-({4-[1-(4-Chlorophenyl)-1H-pyrrolo[2,3-c]pyridin-3-yl]piperidin-1-yl}carbonyl)piperazine;
4-({4-[1-(4-Chlorophenyl)-1H-pyrrolo[2,3-c]pyridin-3-yl]piperidin-1-yl}carbonyl)morpholine;
1-({4-[1-(4-Chlorophenyl)-1H-pyrrolo[2,3-c]pyridin-3-yl]piperidin-1-yl}carbonyl)-1,4-diazepane;
Ethyl 1-[1-(4-chlorophenyl)-1H-pyrazolo[3,4-c]pyridin-3-yl]piperidine-4-carboxylate;
Ethyl 1-[1-(4-methylphenyl)-1H-pyrazolo[3,4-c]pyridin-3-yl]piperidine-4-carboxylate;
1-[1-(4-Chlorophenyl)-1H-pyrazolo[3,4-c]pyridin-3-yl]piperidine-4-carboxylic acid hydrochloride;
N-(2-Aminoethyl)-1-[1-(4-chlorophenyl)-1H-pyrazolo[3,4-c]pyridin-3-yl]piperidine-4-carboxamide dihydrochloride;
-({1-[1-(4-Chlorophenyl)-1H-pyrazolo[3,4-c]pyridin-3-yl]piperidin-4-yl}carbonyl)morpholine;
1-({1-[1-(4-Chlorophenyl)-1H-pyrazolo[3,4-c]pyridin-3-yl]piperidin-4-yl}carbonyl)piperazine dihydrochloride;
{4-[1-(4-Methylphenyl)-1H-pyrazolo[3,4-c]pyridin-3-yl]morpholin-3-yl}methanol;
{4-[1-(4-Methylphenyl)-1H-pyrazolo[3,4-c]pyridin-3-yl]morpholin-2-yl}methanol;
[(3R)-4-[1-(4-Chlorophenyl)-1H-pyrazolo[3,4-c]pyridin-3-yl]morpholin-3-yl]methanol;
Methyl 4-[1-(4-chlorophenyl)-1H-pyrazolo[3,4-c]pyridin-3-yl]morpholine-3-carboxylate;
N-(2-Aminoethyl)-4-[1-(4-chlorophenyl)-1H-pyrazolo[3,4-c]pyridin-3-yl]morpholine-3-carboxamide;
2-{4-[1-(4-Chlorophenyl)-1H-pyrazolo[3,4-c]pyridin-3-yl]morpholin-3-yl}ethan-1-ol;
Methyl 1-[1-(4-chlorophenyl)-1H-pyrazolo[3,4-c]pyridin-3-yl]piperidine-2-carboxylate;
N-(2-Aminoethyl)-1-[1-(4-chlorophenyl)-1H-pyrazolo[3,4-c]pyridin-3-yl]piperidine-2-carboxamide dihydrochloride;
1-({1-[1-(4-Chlorophenyl)-1H-pyrazolo[3,4-c]pyridin-3-yl]piperidin-2-yl}carbonyl)piperazine;
4-[1-(4-Methylphenyl)-1H-pyrrolo[2,3-c]pyridin-3-yl]morpholine;
1-(4-Chlorophenyl)-3-(piperidin-4-yl)-1H-pyrrolo[2,3-c]pyridin-4-ol;
N-Butyl-1-(4-chlorophenyl)-N-methyl-1H-pyrazolo[3,4-c]pyridin-3-amine;
1-[4-(Fluoromethyl)phenyl]-3-(oxan-4-yl)-1H-pyrazolo[3,4-c]pyridine;
3-({4-[1-(4-Chlorophenyl)-1H-pyrazolo[3,4-c]pyridin-3-yl]piperidin-1-yl}methyl)pyridine; or a pharmaceutically acceptable salt thereof.

12. The method of claim 3, wherein the VAP-1 inhibitor compound is:
4-[1-(4-Chlorophenyl)-1H-pyrrolo[2,3-c]pyridin-3-yl]-1-(pyrrolidin-3-yl)piperidine;
4-[1-(4-Chlorophenyl)-1H-pyrrolo[2,3-c]pyridin-3-yl]-1-(piperidin-4-yl)piperidine;
4-[1-(4-Chlorophenyl)-1H-pyrrolo[2,3-c]pyridin-3-yl]-1-(piperidin-4-ylmethyl)piperidine;
1-{4-[1-(4-Chlorophenyl)-1H-pyrrolo[2,3-c]pyridin-3-yl]piperidin-1-yl}-2-(piperidin-4-yl)ethan-1-one;
1-({4-[1-(4-Chlorophenyl)-1H-pyrrolo[2,3-c]pyridin-3-yl]piperidin-1-yl}carbonyl)-4-methylpiperazine;
4-[1-(4-Chlorophenyl)-1H-pyrrolo[2,3-c]pyridin-3-yl]-N-(piperidin-4-ylmethyl)piperidine-1-carboxamide;
4-[1-(4-Chlorophenyl)-1H-pyrrolo[2,3-c]pyridin-3-yl]-N-(piperidin-4-yl)piperidine-1-carboxamide;
4-[1-(4-Chlorophenyl)-1H-pyrrolo[2,3-c]pyridin-3-yl]-N-(1-methylpiperidin-4-yl)piperidine-1-carboxamide;
4-[1-(4-Chlorophenyl)-1H-pyrrolo[2,3-c]pyridin-3-yl]-N-[1-(propan-2-yl)piperidin-4-yl]piperidine-1-carboxamide;
N-(1-Acetylpiperidin-4-yl)-4-[1-(4-chlorophenyl)-1H-pyrrolo[2,3-c]pyridin-3-yl]piperidine-1-carboxamide;
4-[1-(4-Chlorophenyl)-1H-pyrrolo[2,3-c]pyridin-3-yl]-N-[(1-methylpiperidin-4-yl)methyl]piperidine-1-carboxamide;
4-[1-(4-Chlorophenyl)-1H-pyrrolo[2,3-c]pyridin-3-yl]-N-[(1-ethylpiperidin-4-yl)methyl]piperidine-1-carboxamide;
4-[1-(4-Chlorophenyl)-1H-pyrrolo[2,3-c]pyridin-3-yl]-N-methyl-N-[(1-methylpiperidin-4-yl)methyl]piperidine-1-carboxamide formic acid;
N-{[1-(Carbamoylmethyl)piperidin-4-yl]methyl}-4-[1-(4-chlorophenyl)-1H-pyrrolo[2,3-c]pyridin-3-yl]piperidine-1-carboxamide formic acid;
4-[1-(4-Chlorophenyl)-1H-pyrrolo[2,3-c]pyridin-3-yl]-N-methyl-N-{[1-(propan-2-yl)piperidin-4-yl]methyl}piperidine-1-carboxamide;
1-({4-[1-(4-Chlorophenyl)-1H-pyrrolo[2,3-c]pyridin-3-yl]piperidin-1-yl}carbonyl)-4-cyclopropylpiperazine;
1-({4-[1-(4-Chlorophenyl)-1H-pyrrolo[2,3-c]pyridin-3-yl]piperidin-1-yl}carbonyl)-4-(propan-2-yl)piperazine;
1-({4-[1-(4-Chlorophenyl)-1H-pyrrolo[2,3-c]pyridin-3-yl]piperidin-1-yl}carbonyl)-4-(2-methoxyethyl)piperazine;
(3S)-1-({4-[1-(4-Chlorophenyl)-1H-pyrrolo[2,3-c]pyridin-3-yl]piperidin-1-yl}carbonyl)-3-(propan-2-yl)piperazine;
4-[1-(4-Chlorophenyl)-1H-pyrrolo[2,3-c]pyridin-3-yl]-N-(morpholin-2-ylmethyl)piperidine-1-carboxamide;
4-[1-(4-Chlorophenyl)-1H-pyrrolo[2,3-c]pyridin-3-yl]-N-[(1,4-dimethyl piperazin-2-yl)methyl]piperidine-1-carboxamide;
4-[1-(4-Chlorophenyl)-1H-pyrrolo[2,3-c]pyridin-3-yl]-N-[2-(morpholin-4-yl)ethyl]piperidine-1-carboxamide;
4-[1-(4-Chlorophenyl)-1H-pyrrolo[2,3-c]pyridin-3-yl]-N-[2-(piperazin-1-yl)ethyl]piperidine-1-carboxamide;
4-[1-(4-Chlorophenyl)-1H-pyrrolo[2,3-c]pyridin-3-yl]-N-[2-(1-methylpiperidin-4-yl)ethyl]piperidine-1-carboxamide;
4-[1-(4-Chlorophenyl)-1H-pyrrolo[2,3-c]pyridin-3-yl]-N-[2-(4-methylpiperazin-1-yl)ethyl]piperidine-1-carboxamide;
4-[1-(4-Chlorophenyl)-1H-pyrrolo[2,3-c]pyridin-3-yl]-N-[3-(morpholin-4-yl)propyl]piperidine-1-carboxamide;
4-[1-(4-Chlorophenyl)-1H-pyrrolo[2,3-c]pyridin-3-yl]-N-{[1-(propan-2-yl)piperidin-4-yl]methyl}piperidine-1-carboxamide formic acid;
4-[1-(4-Chlorophenyl)-1H-pyrrolo[2,3-c]pyridin-3-yl]-N-{[1-(2-methoxyethyl)piperidin-4-yl]methyl}piperidine-1-carboxamide formic acid;
N-[3-({4-[1-(4-Chlorophenyl)-1H-pyrrolo[2,3-c]pyridin-3-yl]piperidin-1-yl}carbonylamino)propyl]acetamide;
Propan-2-yl N-({4-[1-(4-methylphenyl)-1H-pyrazolo[3,4-c]pyridin-3-yl]morpholin-2-yl}methyl)carbamate;
3-Cyclopropyl-1-({4-[1-(4-methylphenyl)-1H-pyrazolo[3,4-c]pyridin-3-yl]morpholin-2-yl}methyl)urea;
2-({4-[1-(4-Methylphenyl)-1H-pyrazolo[3,4-c]pyridin-3-yl]morpholin-2-yl}methoxy)ethan-1-amine;
2-Aminoethyl)({4-[1-(4-methylphenyl)-1H-pyrazolo[3,4-c]pyridin-3-yl]morpholin-2-yl}methyl)amine trihydrochloride;
4-[1-(4-Methylphenyl)-1H-pyrazolo[3,4-c]pyridin-3-yl]-2-(morpholin-4-ylmethyl)morpholine;
4-[1-(4-Chlorophenyl)-1H-pyrazolo[3,4-c]pyridin-3-yl]-2-[(4-methylpiperazin-1-yl)methyl]morpholine;
4-[1-(4-Chlorophenyl)-1H-pyrazolo[3,4-c]pyridin-3-yl]-2-(piperazin-1-ylmethyl)morpholine trihydrochloride;
3-Aminopropyl 4-({4-[1-(4-chlorophenyl)-1H-pyrazolo[3,4-c]pyridin-3-yl]morpholin-2-yl}methyl)piperazine-1-carboxylate trihydrochloride;
N-(3-Aminopropyl)-4-({4-[1-(4-chlorophenyl)-1H-pyrazolo[3,4-c]pyridin-3-yl]morpholin-2-yl}methyl)piperazine-1-carboxamide trihydrochloride;
4-({4-[1-(4-Chlorophenyl)-1H-pyrazolo[3,4-c]pyridin-3-yl]morpholin-2-yl}methyl)-N-ethylpiperazine-1-carboxamide;
Methyl 2-{4-[1-(4-chlorophenyl)-1H-pyrazolo[3,4-c]pyridin-3-yl]morpholin-3-yl}acetate;
4-[1-(4-Chlorophenyl)-1H-pyrazolo[3,4-c]pyridin-3-yl]-3-(morpholin-4-ylmethyl)morpholine;
4-[1-(4-Chlorophenyl)-1H-pyrazolo[3,4-c]pyridin-3-yl]-3-[2-(4-methylpiperazin-1-yl)ethyl]morpholine;
1-[1-(4-Chlorophenyl)-1H-pyrazolo[3,4-c]pyridin-3-yl]-N-[(1-methylpiperidin-4-yl)methyl]piperidine-2-carboxamide;
1-(4-Chlorophenyl)-N-[2-(morpholin-4-yl)ethyl]-1H-pyrazolo[3,4-c]pyridin-3-amine;
1-(4-Chlorophenyl)-N-[2-(piperazin-1-yl)ethyl]-1H-pyrazolo[3,4-c]pyridin-3-amine;
1-(4-Chlorophenyl)-N-[2-(4-methylpiperazin-1-yl)ethyl]-1H-pyrazolo[3,4-c]pyridin-3-amine;
1-[1-(4-Chlorophenyl)-1H-pyrazolo[3,4-c]pyridin-3-yl]-N-(piperidin-4-ylmethyl)piperidine-4-carboxamide dihydrochloride;
4-({1-[1-(4-Chlorophenyl)-1H-pyrazolo[3,4-c]pyridin-3-yl]piperidin-4-yl}methyl)morpholine dihydrochloride;
1-({1-[1-(4-Chlorophenyl)-1H-pyrazolo[3,4-c]pyridin-3-yl]piperidin-4-yl}methyl)piperazine;
({1-[1-(4-Chlorophenyl)-1H-pyrazolo[3,4-c]pyridin-3-yl]piperidin-4-yl}methyl)(piperidin-4-ylmethyl)amine;
4-[1-(4-Chlorophenyl)-1H-pyrazolo[3,4-c]pyridin-3-yl]-N-[(1-methylpiperidin-4-yl)methyl]piperazine-1-carboxamide;
1-[1-(4-Methylphenyl)-1H-pyrazolo[3,4-c]pyridin-3-yl]piperidin-4-yl acetate;
2-{4-[1-(4-Chlorophenyl)-1H-pyrazolo[3,4-c]pyridin-3-yl]morpholin-3-yl}acetic acid hydrochloride;
N-(2-Aminoethyl)-2-{4-[1-(4-chlorophenyl)-1H-pyrazolo[3,4-c]pyridin-3-yl]morpholin-3-yl}acetamide dihydrochloride;
2-{4-[1-(4-Chlorophenyl)-1H-pyrazolo[3,4-c]pyridin-3-yl]morpholin-3-yl}-1-(4-methylpiperazin-1-yl)ethan-1-one;
2-{4-[1-(4-Chlorophenyl)-1H-pyrazolo[3,4-c]pyridin-3-yl]morpholin-3-yl}-1-[(3S)-3-(dimethylamino)pyrrolidin-1-yl]ethan-1-one;
2-{4-[1-(4-Chlorophenyl)-1H-pyrazolo[3,4-c]pyridin-3-yl]morpholin-3-yl}-N-(1-methylpiperidin-4-yl)acetamide;
2-{4-[1-(4-Chlorophenyl)-1H-pyrazolo[3,4-c]pyridin-3-yl]morpholin-3-yl}-N-[(1-methylpiperidin-4-yl)methyl]acetamide; or a pharmaceutically acceptable salt thereof.

13. The method of claim 3, wherein the VAP-1 inhibitor compound is:
3-(4-Fluorophenyl)-1-(tetrahydro-2H-pyran-4-yl)-1H-pyrazolo[4,3-c]pyridine;
3-(4-Chlorophenyl)-2-(tetrahydro-2H-pyran-4-yl)-2H-pyrazolo[4,3-c]pyridine;
3-(4-Methylphenyl)-1-(tetrahydro-2H-pyran-4-yl)-1H-pyrazolo[4,3-c]pyridine;
3-(4-Chlorophenyl)-1-[(3R)-tetrahydrofuran-3-yl]-1H-pyrazolo[4,3-c]pyridine;
3-(4-Chlorophenyl)-1-piperidin-4-yl-1H-pyrazolo[4,3-c]pyridine;
-(4-Chlorophenyl)-1-(1-methylpiperidin-4-yl)-1H-pyrazolo[4,3-c]pyridine;
{4-[3-(4-Chlorophenyl)-1H-pyrazolo[4,3-c]pyridin-1-yl]piperidin-1-yl}acetonitrile;
3-(4-Chlorophenyl)-1-(tetrahydro-2H-pyran-4-ylmethyl)-1H-pyrazolo[4,3-c]pyridine;
3-(4-Chlorophenyl)-1-[1-(methylsulfonyl)piperidin-4-yl]-1H-pyrazolo[4,3-c]pyridine;
1-(1-Acetylpiperidin-4-yl)-3-(4-chlorophenyl)-1H-pyrazolo[4,3-c]pyridine;
3-(4-Chlorophenyl)-1-[1-(2-methoxyethyl)piperidin-4-yl]-1H-pyrazolo[4,3-c]pyridine;
3-(4-Chlorophenyl)-1-piperidin-3-yl-1H-pyrazolo[4,3-c]pyridine;
3-(4-Chlorophenyl)-1-[(3S)-tetrahydrofuran-3-yl]-1H-pyrazolo[4,3-c]pyridine;
3-(4-Chlorophenyl)-1-(tetrahydrofuran-3-ylmethyl)-1H-pyrazolo[4,3-c]pyridine;
3-(4-Chlorophenyl)-1-(1-ethylpiperidin-4-yl)-1H-pyrazolo[4,3-c]pyridine;
3-(4-Chlorophenyl)-1-(1-isopropylpiperidin-4-yl)-1H-pyrazolo[4,3-c]pyridine;
-(4-Fluorophenyl)-1-(1-methylpiperidin-4-yl)-1H-pyrazolo[4,3-c]pyridine;
3-(4-Fluorophenyl)-1-piperidin-4-yl-1H-pyrazolo[4,3-c]pyridine;
[1-(Tetrahydro-2H-pyran-4-yl)-1H-pyrazolo[4,3-c]pyridin-3-yl]benzonitrile;
-[1-(1-Methylpiperidin-4-yl)-1H-pyrazolo[4,3-c]pyridin-3-yl]benzonitrile; or a pharmaceutically acceptable salt thereof.

14. The method of claim 3, wherein the VAP-1 inhibitor compound is:
[2-(4-Methylphenyl)imidazo[1,2-a]pyridin-3-yl]methanol;
[2-(2,4-Dichlorophenyl)imidazo[1,2-a]pyridin-3-yl]methanol;
[2-(4-Bromophenyl)-8-methylimidazo[1,2-a]pyridin-3-yl]methanol;
[7-Methyl-2-(4-methylphenyl)imidazo[1,2-a]pyridin-3-yl]methanol;
[2-(4-Bromophenyl)-7-methylimidazo[1,2-a]pyridin-3-yl]methanol;
[2-(4-Bromophenyl)-7-ethylimidazo[1,2-a]pyridin-3-yl]methanol;
[2-(2-Chlorophenyl)-7-methylimidazo[1,2-a]pyridin-3-yl]methanol;
[2-(2,4-Dichlorophenyl)-7-methylimidazo[1,2-a]pyridin-3-yl]methanol;
[2-(3,4-Dichlorophenyl)-7-methylimidazo[1,2-a]pyridin-3-yl]methanol;
[6-Methyl-2-(2-naphthyl)imidazo[1,2-a]pyridin-3-yl]methanol;
[2-(3-Methoxyphenyl)-6-methylimidazo[1,2-a]pyridin-3-yl]methanol;
4-[3-(Hydroxymethyl)-6-methylimidazo[1,2-a]pyridin-2-yl]benzonitrile;
[6-Methyl-2-(3-nitrophenyl)imidazo[1,2-a]pyridin-3-yl]methanol;
[2-(4-Chlorophenyl)-6-methylimidazo[1,2-a]pyridin-3-yl]methanol;
2-(4-Fluorophenyl)-6-methylimidazo[1,2-a]pyridin-3-yl]methanol;
[2-(4-Iodophenyl)-6-methylimidazo[1,2-a]pyridin-3-yl]methanol;
[2-(2-Chlorophenyl)-6-methylimidazo[1,2-a]pyridin-3-yl]methanol;
(2-{4-[(2-Aminoethyl)amino]phenyl}-6-methylimidazo[1,2-a]pyridin-3-yl)methanol;
1-[2-(4-Chlorophenyl)-6-methylimidazo[1,2-a]pyridin-3-yl]ethanol;
[2-(2,4-Dichlorophenyl)-6-methylimidazo[1,2-a]pyridin-3-yl]methanol;
[2-(3-Methoxyphenyl)-6-(trifluoromethyl)imidazo[1,2-a]pyridin-3-yl]methanol;
[2-(4-Chlorophenyl)-6-(trifluoromethyl)imidazo[1,2-a]pyridin-3-yl]methanol;
[2-(4-Bromophenyl)-6-(trifluoromethyl)imidazo[1,2-a]pyridin-3-yl]methanol;
[7-Chloro-2-(4-chlorophenyl)imidazo[1,2-a]pyridin-3-yl]methanol;
[2-(4-Bromophenyl)-7-chloroimidazo[1,2-a]pyridin-3-yl]methanol trifluoroacetate;
[7-Chloro-2-(2,4-dichlorophenyl)imidazo[1,2-a]pyridin-3-yl]methanol;
[7-Chloro-2-(2,4-difluorophenyl)imidazo[1,2-a]pyridin-3-yl]methanol;
[6-Bromo-2-(3-methoxyphenyl)imidazo[1,2-a]pyridin-3-yl]methanol;
6-Chloro-2-(4-chlorophenyl)imidazo[1,2-a]pyridin-3-yl]methanol;
[6-Bromo-2-(4-fluorophenyl)imidazo[1,2-a]pyridin-3-yl]methanol;
[6-Bromo-2-(4-bromophenyl)imidazo[1,2-a]pyridin-3-yl]methanol trifluoroacetate;
[2-(4-Bromophenyl)-6-chloroimidazo[1,2-a]pyridin-3-yl]methanol trifluoroacetate;
[2-(4-Chlorophenyl)-6-fluoroimidazo[1,2-a]pyridin-3-yl]methanol;
[6-Bromo-2-(2,4-difluorophenyl)imidazo[1,2-a]pyridin-3-yl]methanol;
[6-Chloro-2-(2,4-difluorophenyl)imidazo[1,2-a]pyridin-3-yl]methanol;
[6-Bromo-2-(2,4-dichlorophenyl)imidazo[1,2-a]pyridin-3-yl]methanol;
[6-Chloro-2-(2,4-dichlorophenyl)imidazo[1,2-a]pyridin-3-yl]methanol;
[6-Bromo-2-(3,4-difluorophenyl)imidazo[1,2-a]pyridin-3-yl]methanol;
[6-Bromo-2-(3-chloro-4-fluorophenyl)imidazo[1,2-a]pyridin-3-yl]methanol;
[6-Chloro-2-(3-chloro-4-fluorophenyl)imidazo[1,2-a]pyridin-3-yl]methanol;
6,8-Dichloro-2-(3-methoxyphenyl)imidazo[1,2-a]pyridin-3-yl]methanol;
[2-(4-Bromophenyl)-6,8-dichloroimidazo[1,2-a]pyridin-3-yl]methanol;
2-(4-Bromophenyl)-3-(hydroxymethyl)imidazo[1,2-a]pyridine-6-carbonitrile;
Methyl 2-(4-bromophenyl)-3-(hydroxymethyl)imidazo[1,2-a]pyridine-6-carboxylate;
Methyl 2-(4-chlorophenyl)-3-(hydroxymethyl)imidazo[1,2-a]pyridine-6-carboxylate hydrobromide;
[2-(4-Bromophenyl)imidazo[1,2-a]pyridine-3,7-diyl]dimethanol;
[2-(4-Chlorophenyl)imidazo[1,2-a]pyridine-3,6-diyl]dimethanol;
[2-(4-Chlorophenyl)-6-nitroimidazo[1,2-a]pyridin-3-yl]methanol;
[2-(4-Bromophenyl)-6-nitroimidazo[1,2-a]pyridin-3-yl]methanol hydrochloride;
{2-(4-Chlorophenyl)-6-[(4-methoxypiperidin-1-yl)carbonyl]imidazo[1,2-a]pyridin-3-yl}methanol;
2-(4-Chlorophenyl)-3-(hydroxymethyl)-N-(3-methoxypropyl)imidazo[1,2-a]pyridine-6-carboxamide;
2-(4-Chlorophenyl)-3-(hydroxymethyl)-N-(2-methoxyethyl)imidazo[1,2-a]pyridine-6-carboxamide;
[2-(4-Chlorophenyl)-6-(morpholin-4-ylcarbonyl)imidazo[1,2-a]pyridin-3-yl]methanol;
2-(4-Chlorophenyl)-3-(hydroxymethyl)-N,N-dimethylimidazo[1,2-a]pyridine-6-carboxamide;
2-(4-Chlorophenyl)-3-(hydroxymethyl)-N-methylimidazo[1,2-a]pyridine-6-carboxamide;
2-(4-Chlorophenyl)-3-(hydroxymethyl)-N-[2-(1-methylpyrrolidin-2-yl)ethyl]imidazo[1,2-a]pyridine-6-carboxamide;
{2-(4-Chlorophenyl)-6-[(4-methylpiperazin-1-yl)carbonyl]imidazo[1,2-a]pyridin-3-yl}methanol;
2-(4-Chlorophenyl)-N-(3,4-dimethoxybenzyl)-3-(hydroxymethyl)imidazo[1,2-a]pyridine-6-carboxamide;
2-(4-Chlorophenyl)-3-(hydroxymethyl)-N-[2-(1H-imidazol-4-yl)ethyl]imidazo[1,2-a]pyridine-6-carboxamide;
2-(4-Chlorophenyl)-3-(hydroxymethyl)-N-(pyridin-3-ylmethyl)imidazo[1,2-a]pyridine-6-carboxamide;
-(4-Chlorophenyl)-3-(hydroxymethyl)-N-(3-hydroxypropyl)imidazo[1,2-a]pyridine-6-carboxamide;
(1-{[2-(4-Chlorophenyl)-3-(hydroxymethyl)imidazo[1,2-a]pyridin-6-yl]carbonyl}piperidin-4-yl)methanol;
2-(4-Chlorophenyl)-3-(hydroxymethyl)-N-(2-hydroxypropyl)imidazo[1,2-a]pyridine-6-carboxamide;
2-(4-Chlorophenyl)-N-(trans-4-hydroxycyclohexyl)-3-(hydroxymethyl)imidazo[1,2-a]pyridine-6-carboxamide;
1-{[2-(4-Chlorophenyl)-3-(hydroxymethyl)imidazo[1,2-a]pyridin-6-yl]carbonyl}piperidin-4-ol;
(3R)-1-{[2-(4-Chlorophenyl)-3-(hydroxymethyl)imidazo[1,2-a]pyridin-6-yl]carbonyl}pyrrolidin-3-ol;
1-{[2-(4-Chlorophenyl)-3-(hydroxymethyl)imidazo[1,2-a]pyridin-6-yl]carbonyl}pyrrolidin-3-ol;
1-{[2-(4-Chlorophenyl)-3-(hydroxymethyl)imidazo[1,2-a]pyridin-6-yl]carbonyl}azetidin-3-ol;
2-(4-Chlorophenyl)-3-(hydroxymethyl)imidazo[1,2-a]pyridine-7-carboxamide;
3-(Hydroxymethyl)-2-(3-methoxyphenyl)imidazo[1,2-a]pyridine-6-carboxamide;
2-(4-Chlorophenyl)-3-(hydroxymethyl)imidazo[1,2-a]pyridine-6-carboxamide;
-(4-Fluorophenyl)-3-(hydroxymethyl)imidazo[1,2-a]pyridine-6-carboxamide;
2-(2,4-Difluorophenyl)-3-(hydroxymethyl)imidazo[1,2-a]pyridine-6-carboxamide;
2-(2,4-Dichlorophenyl)-3-(hydroxymethyl)imidazo[1,2-a]pyridine-6-carboxamide;
2-(3,4-Difluorophenyl)-3-(hydroxymethyl)imidazo[1,2-a]pyridine-6-carboxamide;
[6-amino-2-(4-chlorophenyl)imidazo[1,2-a]pyridin-3-yl]methanol;
N-[2-(4-chlorophenyl)-3-(hydroxymethyl)imidazo[1,2-a]pyridin-6-yl]acetamide;
[6-amino-2-(4-bromophenyl)imidazo[1,2-a]pyridin-3-yl]methanol;
[6-chloro-2-(4-chlorophenyl)imidazo[1,2-b]pyridazin-3-yl]methanol;
[2-(4-Chlorophenyl)imidazo[1,2-a]pyrazin-3-yl]methanol;
[6-Bromo-2-(3-methoxyphenyl)imidazo[1,2-a]pyrazin-3-yl]methanol;
{6-Bromo-2-[4-(trifluoromethyl)phenyl]imidazo[1,2-a]pyrazin-3-yl}methanol;
[6-Bromo-2-(4-fluorophenyl)imidazo[1,2-a]pyrazin-3-yl]methanol;
[6-Bromo-2-(4-chlorophenyl)imidazo[1,2-a]pyrazin-3-yl]methanol;
[6-Bromo-2-(4-bromophenyl)imidazo[1,2-a]pyrazin-3-yl]methanol;
[6-Bromo-2-(2,4-dichlorophenyl)imidazo[1,2-a]pyrazin-3-yl]methanol;
[6-Bromo-2-(2,4-difluorophenyl)imidazo[1,2-a]pyrazin-3-yl]methanol;
[6-Bromo-2-(4-chloro-2-fluoro-5-methylphenyl)imidazo[1,2-a]pyrazin-3-yl]methanol;
[2-(1-Benzofuran-5-yl)-6-bromoimidazo[1,2-a]pyrazin-3-yl]methanol;
[6-Bromo-2-(2,3-dihydro-1,4-benzodioxin-5-yl)imidazo[1,2-a]pyrazin-3-yl]methanol;
[6-amino-2-(4-fluorophenyl)imidazo[1,2-a]pyrazin-3-yl]methanol;
[6-amino-2-(4-chlorophenyl)imidazo[1,2-a]pyrazin-3-yl]methanol;
[6-Amino-2-(4-bromophenyl)imidazo[1,2-a]pyrazin-3-yl]methanol;
[6-(Azetidin-1-yl)-2-(4-fluorophenyl)imidazo[1,2-a]pyrazin-3-yl]methanol;
[2-(4-Chlorophenyl)imidazo[1,2-a]pyrimidin-3-yl]methanol;
[2-(2,4-Dichlorophenyl)imidazo[1,2-a]pyrimidin-3-yl]methanol;
[6-(4-fluorophenyl)-2-methylimidazo[2,1-b][1,3]oxazol-5-yl]methanol;
[6-(4-Chlorophenyl)imidazo[2,1-b][1,3]thiazol-5-yl]methanol;
[6-(4-Bromophenyl)imidazo[2,1-b][1,3]thiazol-5-yl]methanol;
[6-(2,4-dichlorophenyl)imidazo[2,1-b][1,3]thiazol-5-yl]methanol;
[6-(4-Bromophenyl)-2-methylimidazo[2,1-b][1,3]thiazol-5-yl]methanol;
[6-(2,4-Dichlorophenyl)-2-methylimidazo[2,1-b][1,3]thiazol-5-yl]methanol;
[2-Chloro-6-(4-chlorophenyl)imidazo[2,1-b][1,3]thiazol-5-yl]methanol;
Methyl 6-(4-chlorophenyl)-5-(hydroxymethyl)imidazo[2,1-b][1,3]thiazole-2-carboxylate;
[6-(4-Chlorophenyl)imidazo[2,1-b][1,3]thiazole-2,5-diyl]dimethanol;
1-[6-(4-Chlorophenyl)-5-(hydroxymethyl)imidazo[2,1-b][1,3]thiazol-2-yl]ethanol;
[6-(4-Chlorophenyl)-5-(hydroxymethyl)imidazo[2,1-b][1,3]thiazol-2-yl](cyclopropyl)methanol;
2-[6-(4-Chlorophenyl)-5-(hydroxymethyl)imidazo[2,1-b][1,3]thiazol-2-yl]propan-2-ol;
6-(4-Chlorophenyl)-N-ethyl-5-(hydroxymethyl)-N-methylimidazo[2,1-b][1,3]thiazole-2-carboxamide;
[6-(4-Chlorophenyl)-2-(morpholin-4-ylcarbonyl)imidazo[2,1-b][1,3]thiazol-5-yl]methanol;
{6-(4-Chlorophenyl)-2-[(4-methylpiperazin-1-yl)carbonyl]imidazo[2,1-b][1,3]thiazol-5-yl}methanol;
6-(4-Chlorophenyl)-5-(hydroxymethyl)-N-propylimidazo[2,1-b][1,3]thiazole-2-carboxamide; or a pharmaceutically acceptable salt thereof.

15. The method of claim 3, wherein the VAP-1 inhibitor compound is:
tert-Butyl N-(3-{4-[1-(4-chlorophenyl)-1H-pyrrolo[2,3-c]pyridin-3-yl]piperidin-1-yl}-3-oxopropyl)carbamate;
1-{4-[1-(4-Chlorophenyl)-1H-pyrrolo[2,3-c]pyridin-3-yl]piperidin-1-yl}-2-(dimethyl-amino)ethan-1-one;
1-{4-[1-(4-Chlorophenyl)-1H-pyrrolo[2,3-c]pyridine-3-yl]piperidin-1-yl}-2-hydroxyethan-1-one;
2-Amino-1-{4-[1-(4-chlorophenyl)-1H-pyrrolo[2,3-c]pyridin-3-yl]piperidin-1-yl}ethan-1-one;
3-Amino-1-{4-[1-(4-chlorophenyl)-1H-pyrrolo[2,3-c]pyridin-3-yl]piperidin-1-yl}propan-1-one;
2-{4-[1-(4-Chlorophenyl)-1H-pyrrolo[2,3-c]pyridin-3-yl]piperidin-1-yl}ethan-1-ol;
4-[1-(4-Chlorophenyl)-1H-pyrrolo[2,3-c]pyridin-3-yl]-1-(1H-pyrazol-3-ylmethyl)-piperidine;
4-[1-(4-Chlorophenyl)-1H-pyrrolo[2,3-c]pyridin-3-yl]-1-[(1-methyl-1H-pyrazol-4-yl)-methyl]piperidine;
3-{4-[1-(4-Chlorophenyl)-1H-pyrrolo[2,3-c]pyridine-3-yl]piperidin-1-yl}propanenitrile;
4-{4-[1-(4-Chlorophenyl)-1H-pyrrolo[2,3-c]pyridin-3-yl]piperidin-1-yl}butanenitrile;
[1-(4-Chlorophenyl)-1H-pyrrolo[2,3-c]pyridin-3-yl]methanol;
1-(4-Chlorophenyl)-1H-pyrrolo[2,3-c]pyridine-3-carbaldehyde;
1-{[1-(4-Chlorophenyl)-1H-pyrrolo[2,3-c]pyridin-3-yl]methyl}-4-methylpiperazine;
tert-Butyl 4-{[1-(4-chlorophenyl)-1H-pyrrolo[2,3-c]pyridin-3-yl];
1-{[1-(4-Chlorophenyl)-1H-pyrrolo[2,3-c]pyridin-3-yl]methyl}piperazine;
2-(1-{[1-(4-Chlorophenyl)-1H-pyrrolo[2,3-c]pyridin-3-yl]methyl}piperidin-4-yl)ethan-1-ol;
(1-{[1-(4-Chlorophenyl)-1H-pyrrolo[2,3-c]pyridin-3-yl]methyl}piperidin-4-yl)methanol;
4-{[1-(4-Chlorophenyl)-1H-pyrrolo[2,3-c]pyridin-3-yl]methyl}morpholine;
1-{[1-(4-Chlorophenyl)-1H-pyrrolo[2,3-c]pyridin-3-yl]methyl}piperidin-4-ol;
2-({[1-(4-Chlorophenyl)-1H-pyrrolo[2,3-c]pyridin-3-yl]methyl}amino)ethan-1-ol;
4-[3-(4-Methylphenyl)imidazo[1,5-a]pyrazin-1-yl]morpholine;
4-[3-(4-Chlorophenyl)imidazo[1,5-a]pyrazin-1-yl]morpholine;
3-(4-Chlorophenyl)-N-(2-methoxyethyl)-N-methylimidazo[1,5-a]pyrazin-1-amine;
3-(4-Chlorophenyl)-N,N-dimethylimidazo[1,5-a]pyrazin-1-amine;
3-(4-Chlorophenyl)-1-(oxan-4-yl)imidazo[1,5-a]pyrazine;
3-(4-Chlorophenyl)-1-(oxan-4-ylmethyl)imidazo[1,5-a]pyrazine;
3-(4-Chlorophenyl)-1-(oxolan-3-yl)imidazo[1,5-a]pyrazine;
3-(4-Chlorophenyl)-1-(4-methoxycyclohexyl)imidazo[1,5-a]pyrazine;
4-[3-(4-Chlorophenyl)-4H,5H,6H,7H-imidazo[1,5-a]pyrazin-1-yl]morpholine;
4-[3-(4-Methylphenyl)-4H,5H,6H,7H-imidazo[1,5-a]pyrazin-1-yl]morpholine;
1-[3-(4-Methylphenyl)-1-(morpholin-4-yl)-4H,5H,6H,7H-imidazo[1,5-a]pyrazin-5-yl]ethan-1-one;
1-[3-(4-Methylphenyl)-1-(morpholin-4-yl)-4H,5H,6H,7H-imidazo[1,5-a]pyrazin-5-yl]propan-1-one;
Methyl 3-(4-methylphenyl)-1-(morpholin-4-yl)-4H,5H,6H,7H-imidazo[1,5-a]pyrazine-5-carboxylate;
2,2,2-Trifluoro-1-[3-(4-methylphenyl)-1-(morpholin-4-yl)-4H,5H,6H,7H-imidazo[1,5-a]pyrazin-5-yl]ethan-1-one; or a pharmaceutically acceptable salt thereof.

16. The method according to claim 3 wherein the VAP-1 inhibitor compound is procarbazine, isocarboxazid, guanabenx, carbidopa, benserazide, or a pharmaceutically acceptable salt thereof.

17. The method according to claim 16 wherein the VAP-1 inhibitor compound is carbidopa, benserazide, or a pharmaceutically acceptable salt thereof.
